(19) United States
(12) Patent Application Publication
(10) Pub. No.: US 2023/0118688 A1
(43) Pub. Date: Apr. 20, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Heng CHENG, Foster City, CA (US); Christine M. TARBY, Lawrenceville, NJ (US); Sanjeev GANGWAR, Foster City, CA (US); Ashvinikumar V. GAVAI, Princeton Junction, NJ (US); Walter L. JOHNSON, San Francisco, CA (US); Yam B. POUDEL, Fremont, CA (US); Prasanna SIVAPRAKASAM, Plainsboro, NJ (US); Patrice GILL, Levittown, PA (US); Andrew F. DONNELL, Branchburg, NJ (US); Murugaiah Andappan Murugaiah SUBBAIAH, Bangalore (IN)

(21) Appl. No.: 17/792,869

(22) PCT Filed: Jan. 26, 2021
(86) PCT No.: PCT/US2021/014975

§ 371 Date: Jul. 14, 2022

Related U.S. Application Data
(60) Provisional application No. 63/057,644, filed on Jul. 28, 2020.
(60) Provisional application No. 62/966,085, filed on Jan. 27, 2020.

(51) Int. Cl. A61K 31/519; C07D 487/04; A61K 39/395

(52) U.S. Cl. CPC A61K 31/519; C07D 487/04; A61K 39/3955

(57) ABSTRACT

Compounds according to formula I are useful as agonists of Toll-like receptor 7 (TLR7). Such compounds can be used in cancer treatment, especially in combination with an anti-cancer immunotherapy agent, or as a vaccine adjuvant.

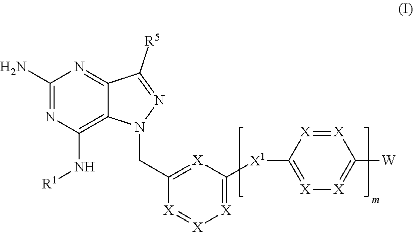

### CROSS-REFERENCE TO RELATED APPLICATIONS

This application claims the benefit under 35 U.S.C. § 119(e) of U.S. Provisional Application Ser. No. 63/057,644, filed Jul. 28, 2020, and U.S. Provisional Application Ser. No. 62/966,085, filed Jan. 27, 2020; the disclosures of which are incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR¹, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

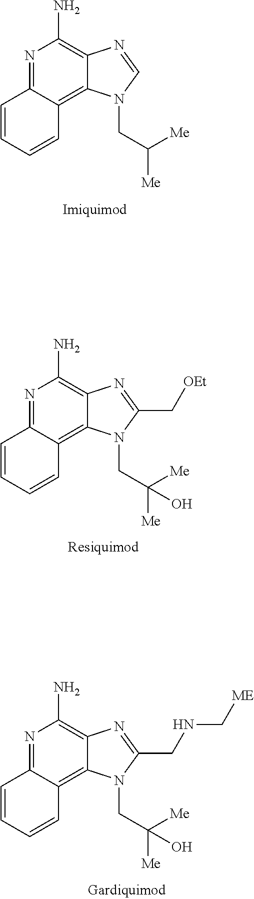

Synthetic TLR7 agonists based on a pteridinone molecular scaffold are also known, as exemplified by vesatolimod (Desai et al. 2015).

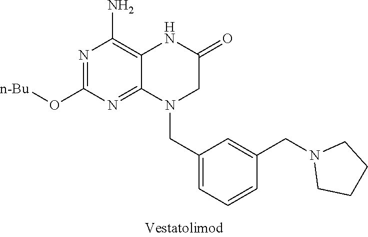

Other synthetic TLR7 agonists based on a purine-like scaffold have been disclosed, frequently according to the general formula (A):

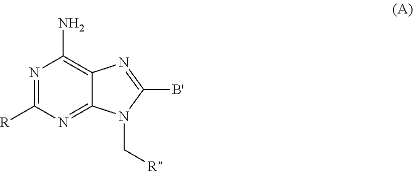

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

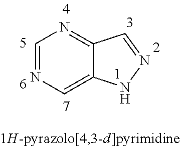

In one aspect, there is provided a compound with a structure according to formula I

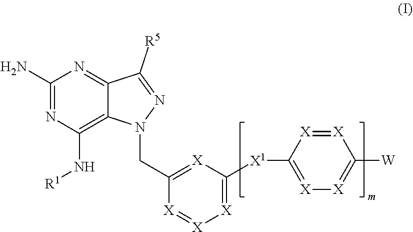

wherein
W is H, halo, C₁-C₃ alkyl, CN, (C₁-C₄ alkanediyl)OH,

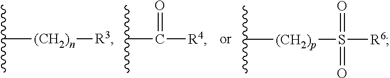

each X is independently N or CR²;
X¹ is O, CH₂, NH, S, or N(C₁-C₃ alkyl);
R¹ is (C₁-C₅ alkyl),
   (C₂-C₅ alkenyl),
   (C₁-C₈ alkanediyl)₀₋₁(C₃-C₆ cycloalkyl),
   (C₁-C₈ alkanediyl)₀₋₁(C₅-C₁₀ spiroalkyl),
   (C₂-C₈ alkanediyl)OH,
   (C₂-C₈ alkanediyl)O(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl),
   or
   (C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R³ is H, halo, OH, CN,
   NH₂,
   NH[C(═O)]₀₋₁(C₁-C₅ alkyl),
   N(C₁-C₅ alkyl)₂,
   NH[C(═O)]₀₋₁(C₁-C₄ alkanediyl)₀₋₁(C₃-C₈ cycloalkyl),
   NH[C(═O)]₀₋₁(C₁-C₄ alkanediyl)₀₋₁(C₄-C₁₀ bicycloalkyl),
   NH[C(═O)]₀₋₁(C₁-C₄ alkanediyl)₀₋₁(C₅-C₁₀ spiroalkyl),
   N(C₃-C₆ cycloalkyl)₂,
   O(C₁-C₄ alkanediyl)₀₋₁(C₃-C₈ cycloalkyl),
   O(C₁-C₄ alkanediyl)₀₋₁(C₄-C₈ bicycloalkyl),
   O(C₁-C₄ alkanediyl)₀₋₁(C₅-C₁₀ spiroalkyl),
   O(C₁-C₄ alkanediyl)₀₋₁(C₁-C₆ alkyl),
   N[C₁-C₃ alkyl]C(═O)(C₁-C₆ alkyl),
   NH(SO₂)(C₁-C₅ alkyl),
   NH(SO₂)(C₁-C₄ alkanediyl)₀₋₁(C₃-C₈ cycloalkyl),
   NH(SO₂)(C₁-C₄ alkanediyl)₀₋₁(C₄-C₁₀ bicycloalkyl),
   NH(SO₂)(C₁-C₄ alkanediyl)₀₋₁(C₅-C₁₀ spiroalkyl),
   a 6-membered aromatic or heteroaromatic moiety,
   a 5-membered heteroaromatic moiety, or
   a moiety having the structure

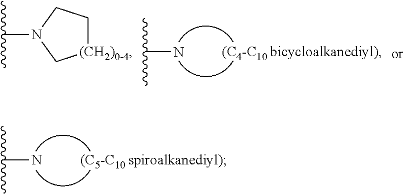

R⁴ is NH₂,
   NH(C₁-C₅ alkyl),
   N(C₁-C₅ alkyl)₂,
   NH(C₁-C₄ alkanediyl)₀₋₁(C₃-C₈ cycloalkyl),
   NH(C₁-C₄ alkanediyl)₀₋₁(C₄-C₁₀ bicycloalkyl),
   NH(C₁-C₄ alkanediyl)₀₋₁(C₅-C₁₀ spiroalkyl),
   N(C₃-C₆ cycloalkyl)₂,
   or
   a moiety having the structure

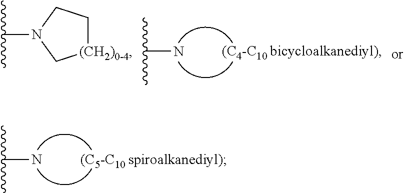

R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

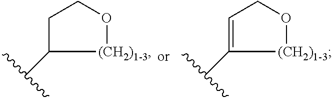

R⁶ is NH₂,
   (NH)₀₋₁(C₁-C₅ alkyl),
   N(C₁-C₅ alkyl)₂,
   (NH)₀₋₁(C₁-C₄ alkanediyl)₀₋₁(C₃-C₈ cycloalkyl),
   (NH)₀₋₁(C₁-C₄ alkanediyl)₀₋₁(C₄-C₁₀ bicycloalkyl),
   (NH)₀₋₁(C₁-C₄ alkanediyl)₀₋₁(C₅-C₁₀ spiroalkyl),
   N(C₃-C₆ cycloalkyl)₂,
   or
   a moiety having the structure

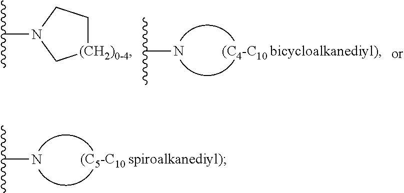

R^{x} and R^{y} are independently H or C₁-C₃ alkyl or R^{x} and R^{y} combine with the nitrogen to which they are bonded to form a 3- to 7-membered heterocycle;
m is 0 or 1;
n is 1, 2, or 3;
and
p is 0, 1, 2, or 3;
wherein in R¹, R², R³, R⁴, R⁶, and R⁶
   an alkyl, cycloalkyl, alkanediyl, bicycloalkyl, spiroalkyl, cyclic amine, 6-membered aromatic or heteroaromatic moiety, 5-membered heteroaromatic moiety or a moiety of the formula

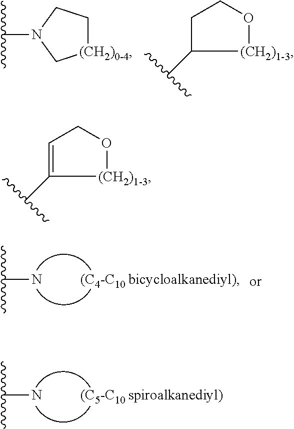

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, bicycloalkyl, spiroalkyl, or a moiety of the formula

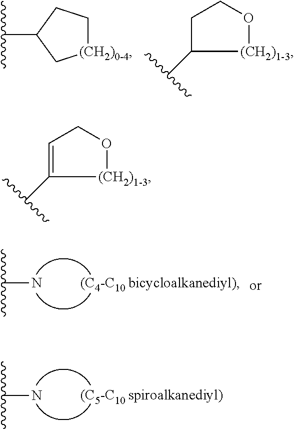

may have a CH₂ group replaced by 0, SO₂, CF₂, C(═O), NH,
N[C(═O)]₀₋₁(C₁-C₃ alkyl),
N[C(═O)]₀₋₁(C₁-C₄ alkanediyl)₀₋₁CF₃,
or
N[C(═O)]₀₋₁(C₁-C₄ alkanediyl)₀₋₁(C₃-C₈ cycloalkyl).

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one embodiment of formula (I), m is 0.

In one aspect, in formula (I), one X is N and the others are CR² in the moiety

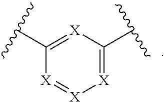

In another aspect, in formula (I) the moiety

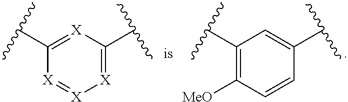

In another aspect, in formula (I) the moiety

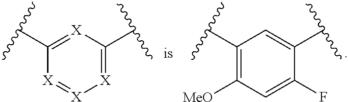

In another aspect in formula (I) the moiety

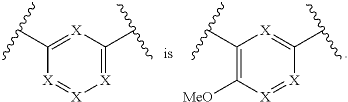

wherein one X is N and the other two are CH.

In one aspect, W is

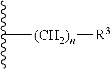

(preferably with n equals 1) or

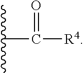

In one aspect, compounds of this disclosure are according to formula (I′), wherein R¹, R⁵, X, and W are as defined in respect of formula (I):

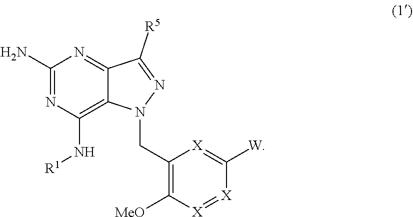

In one aspect, compounds of this disclosure are according to formula (Ia), wherein R¹, R⁵, and W are as defined in respect of formula (I):

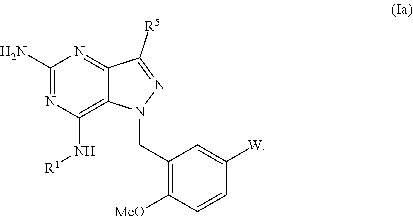

In another aspect, compounds of this disclosure are according to formula (Ib), wherein R¹, R⁵, and R³ are as defined in respect of formula (I):

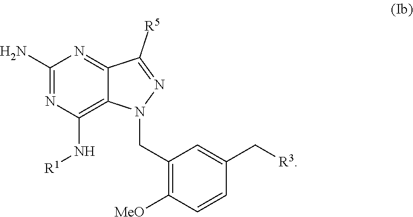

Exemplary embodiments of R² include H, OMe, OCHF₂, and OCF₃, with OMe being a preferred embodiment.

In one embodiment of compounds according to formula (Ib), R³ is
NH(C₁-C₅ alkyl),
N(C₁-C₅ alkyl)₂,
NH(C₁-C₄ alkanediyl)₀₋₁(C₃-C₈ cycloalkyl),
NH(C₁-C₄ alkanediyl)₀₋₁(C₄-C₁₀ bicycloalkyl),
NH(C₁-C₄ alkanediyl)₀₋₁(C₅-C₁₀ spiroalkyl),
N(C₃-C₆ cycloalkyl)₂,
N[C₁-C₃ alkyl](C₁-C₆ alkyl),
or
a cyclic amine moiety having the structure

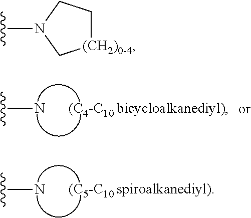

In another embodiment of compounds according to formula (Ib), R³ is
NH[C(═O)](C₁-C₅ alkyl),
NH[C(═O)](C₁-C₄ alkanediyl)₀₋₁(C₃-C₈ cycloalkyl),
NH[C(═O)](C₁-C₄ alkanediyl)₀₋₁(C₄-C₁₀ bicycloalkyl),
or
NH[C(═O)](C₁-C₄ alkanediyl)₀₋₁(C₅-C₁₀ spiroalkyl).

Exemplary embodiments of R⁵ include H, Me, OMe, CH₂OH, cyclopropyl, F, Cl, and CF₃, with H being a preferred embodiment.

In another embodiment of compounds according to formula (Ib), R³ is
O(C₁-C₄ alkanediyl)₀₋₁(C₃-C₈ cycloalkyl),
O(C₁-C₄ alkanediyl)₀₋₁(C₄-C₅ bicycloalkyl),
O(C₁-C₄ alkanediyl)₀₋₁(C₅-C₁₀ spiroalkyl),
or
O(C₁-C₄ alkanediyl)₀₋₁(C₁-C₆ alkyl).

In another aspect, compounds of this disclosure are according to formula (Ic), wherein R³ and R⁵ are as defined in respect of formula (I):

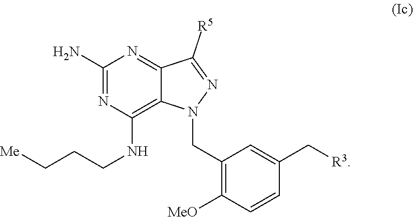

In another aspect, compounds of this disclosure are according to formula (Id), wherein R³ and R⁵ are as defined in respect of formula (I):

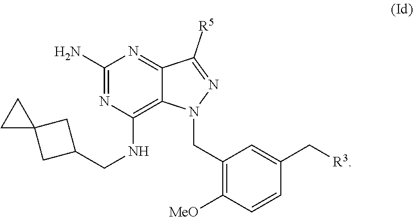

In another aspect, compounds of this disclosure are according to formula (Ie), wherein R¹, R⁴ and R⁵ are as defined in respect of formula (I):

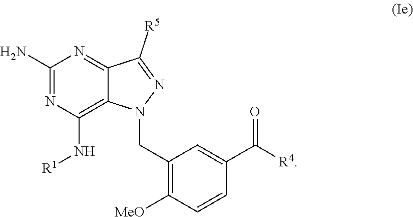

In another aspect, this disclosure provides a compound having a structure according to formula (If)

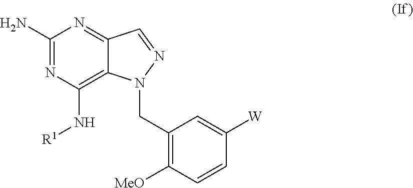

wherein
R¹ is

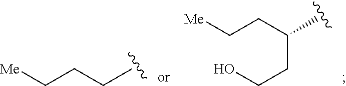

and
W is

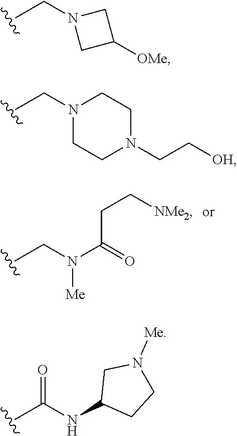

In another aspect, this disclosure provides a compound having a structure according to formula (Ig):

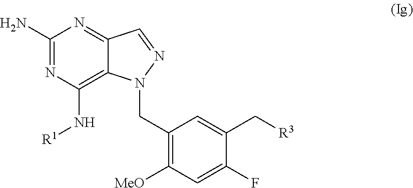

wherein R¹ and R³ are as defined in respect of formula (I).

In another aspect, this disclosure provides a compound having a structure according to formula (Ih):

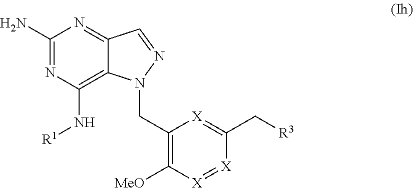

wherein one X is N and the other two are CH and R¹ and R³ are as defined in respect of formula (I).

Examples of groups R¹ are

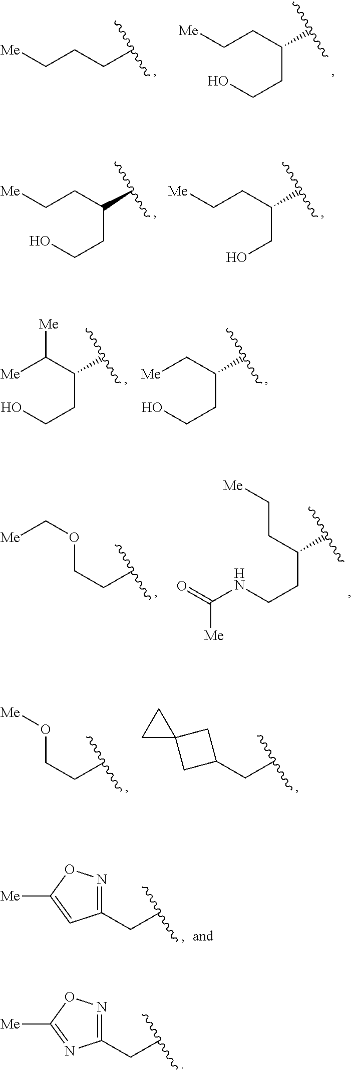

Preferably, R¹ is selected from the following group (“preferred R¹ group”), consisting of

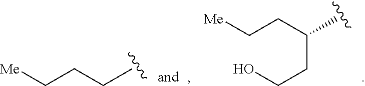

Exemplary groups R³ include

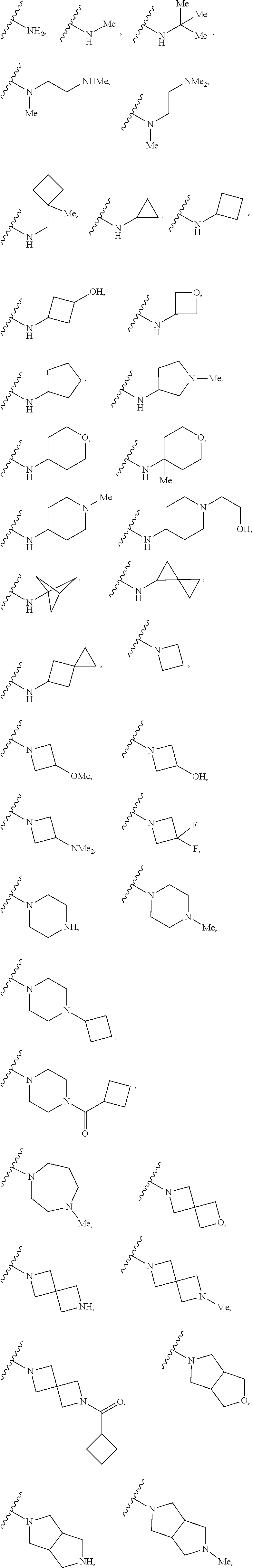

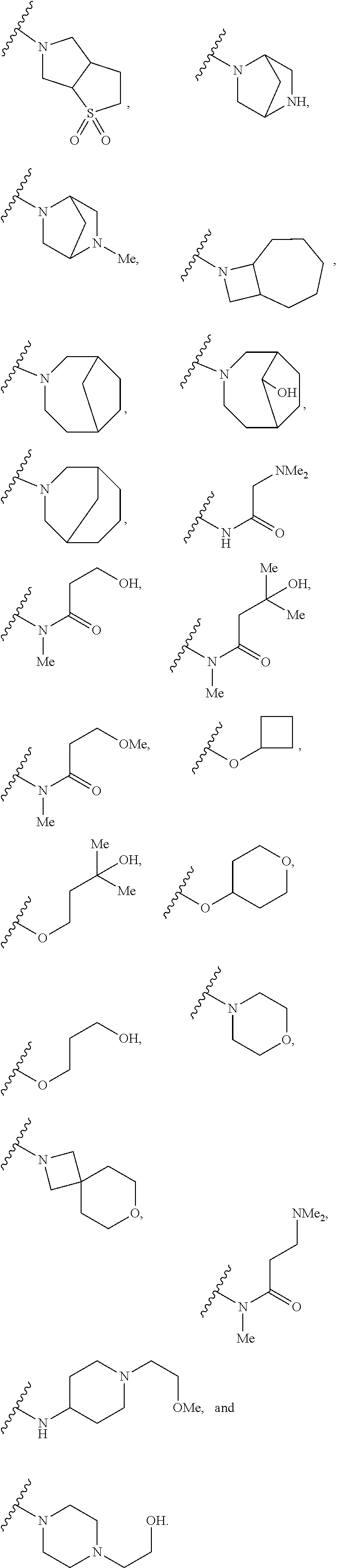

In another aspect,
R³ is H, halo, OH, CN,
   NH₂,
   NH[C(═O)]₀₋₁(C₁-C₅ alkyl),
   N(C₁-C₅ alkyl)₂,
   NH[C(═O)]₀₋₁(C₁-C₄ alkanediyl)₀₋₁(C₃-C₈ cycloalkyl),
   NH[C(═O)]₀₋₁(C₁-C₄ alkanediyl)₀₋₁(C₄-C₁₀ bicycloalkyl),
   NH[C(═O)]₀₋₁(C₁-C₄ alkanediyl)₀₋₁(C₅-C₁₀ spiroalkyl),
   N(C₃-C₆ cycloalkyl)₂,
   N[C₁-C₃ alkyl]C(═O)(C₁-C₆ alkyl),
   a 6-membered aromatic or heteroaromatic moiety,
   a 5-membered heteroaromatic moiety, or
   a moiety having the structure

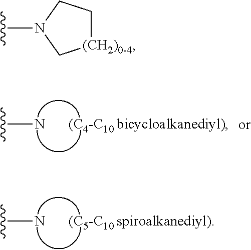

Preferred groups R³ are

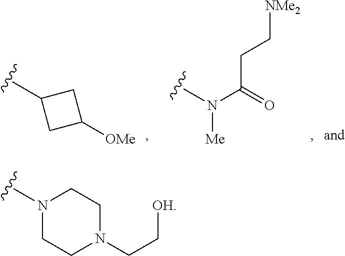

Exemplary groups R⁴ include:

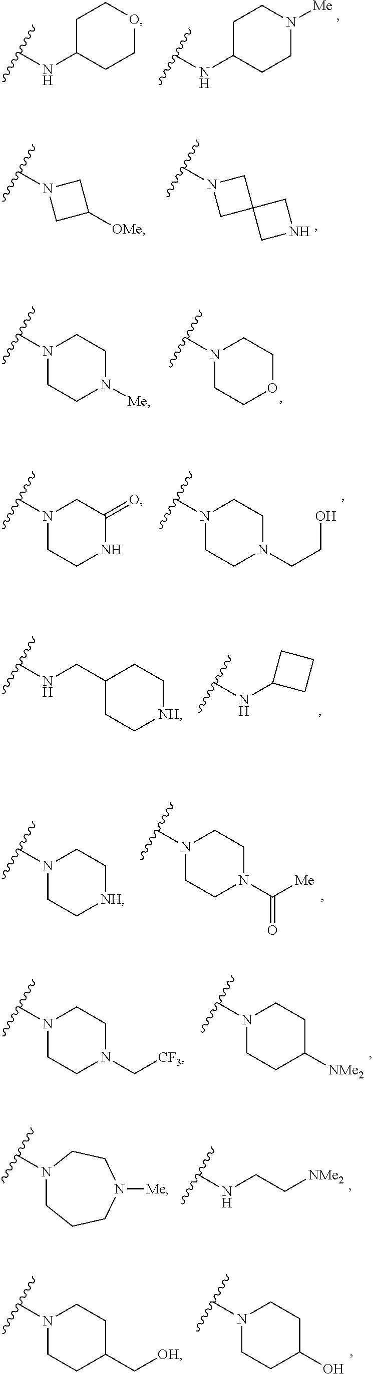

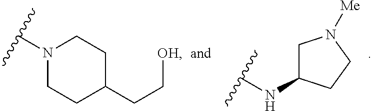 [SMILES: *N1CCC(CCO)CC1 ; *N[C@@H]1CCN(C)C1]

A preferred R⁴ is

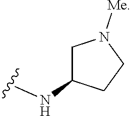

Exemplary groups, R⁵ are H,

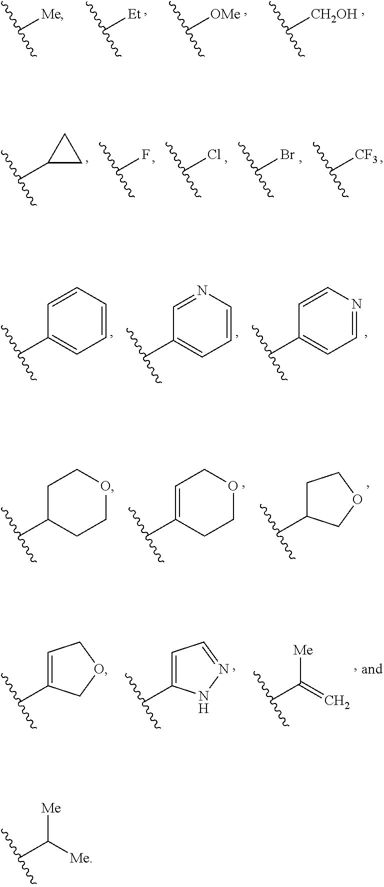

Preferably, R⁵ is H or Me.

By way of exemplification and not of limitation, moieties of the formula

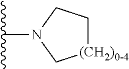 [SMILES: *N1CCCC1]

include

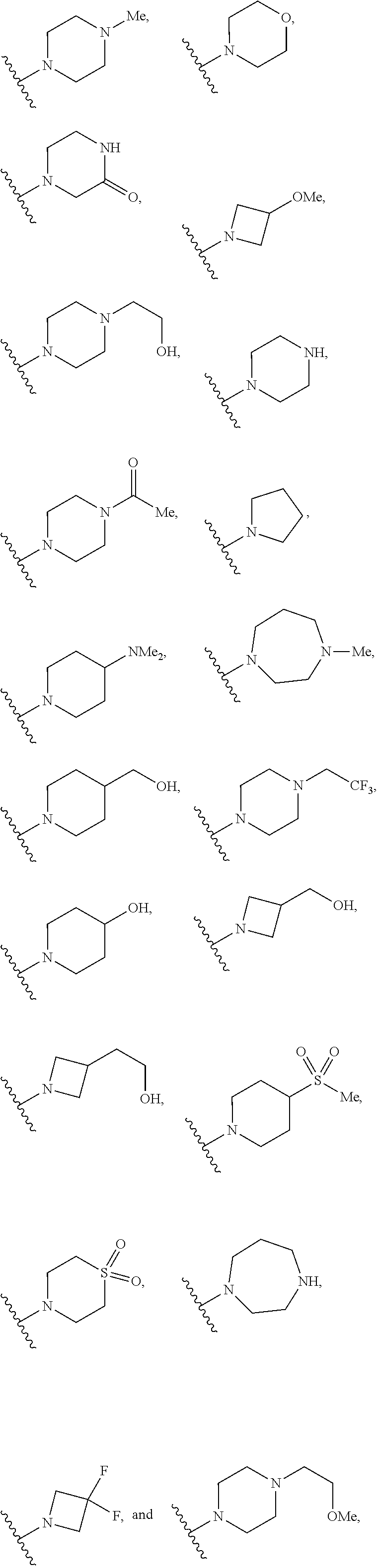

By way of exemplification and not of limitation, spiroalkyl groups include

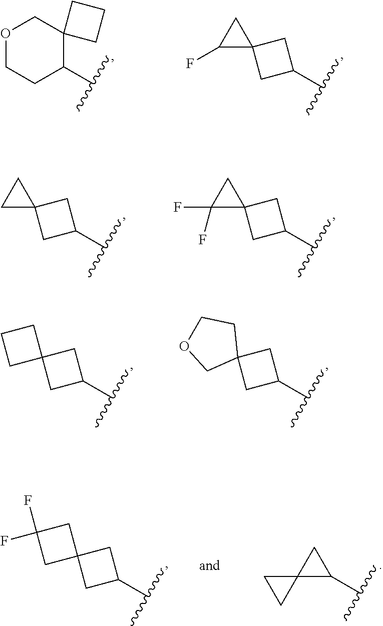

By way of exemplification and not of limitation, moieties of the formula

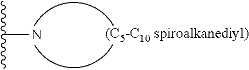 [SMILES: *N]

include

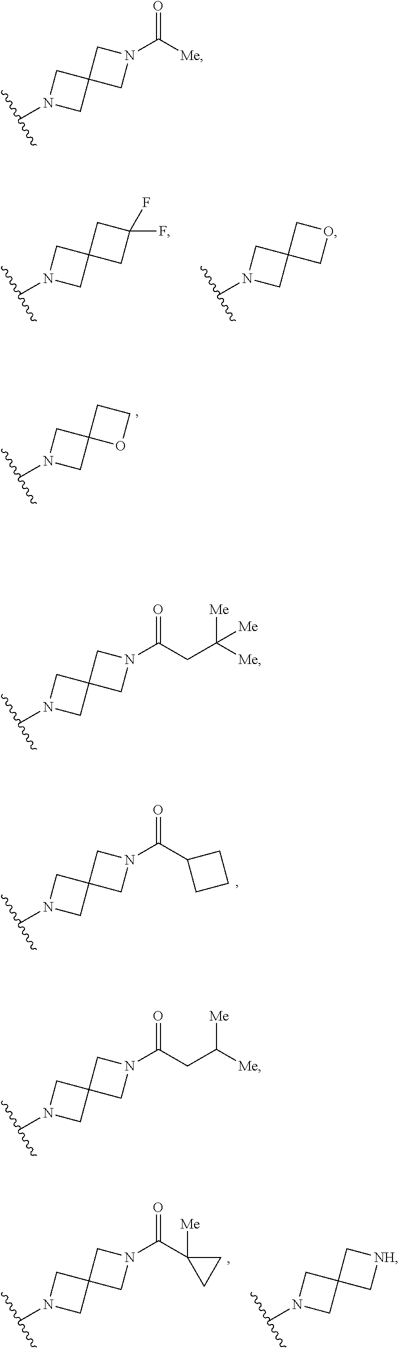

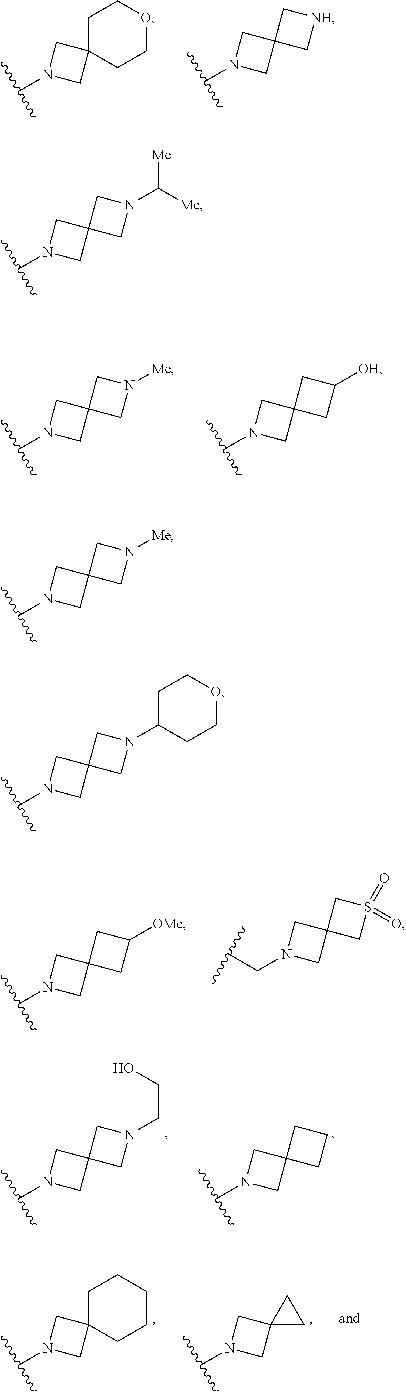

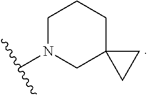 [SMILES: *N1CCCC2(CC2)C1]

By way of exemplification and not of limitation, bicycloalkyl groups include

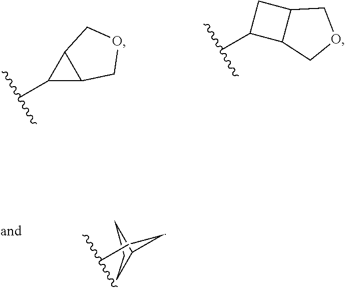 [SMILES: *C1C2COCC12 ; *C1CC2COCC12 ; *CCC1CCC1]

By way of exemplification and not of limitation, moieties of the formula

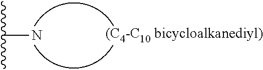 [SMILES: *N]

include

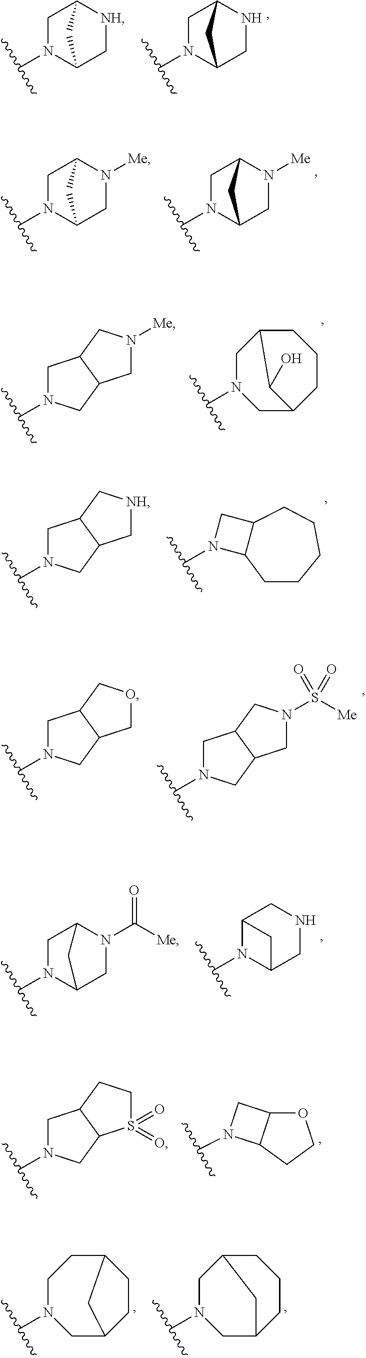

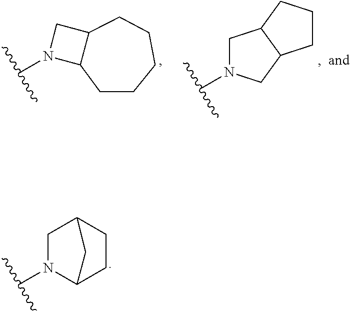 [SMILES: *N1CC2CCCCCC21 ; *N1CC2CCCC2C1 ; *N1CC2CCC1C2]

In one aspect, W is, preferably in combination with formula (I′), (Ia), (If), or (Ig),

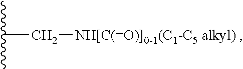

especially

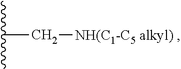

with specific exemplary embodiments being

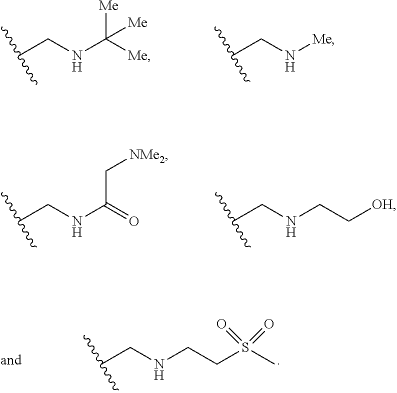

In one aspect, W is, preferably in combination with formula (I′), (Ia), (If), or (Ig),

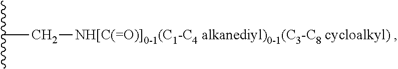

especially

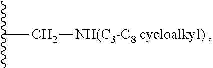

with specific exemplary embodiments being

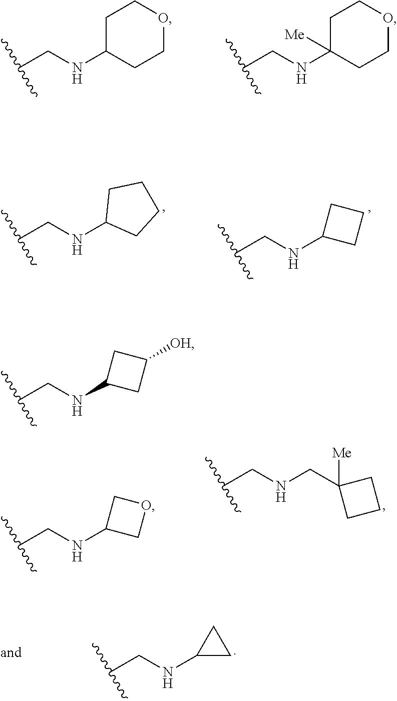

In one aspect, W is, preferably in combination with formula (I′), (Ia), (If), or (Ig),

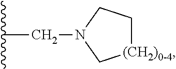

with specific exemplary embodiments being

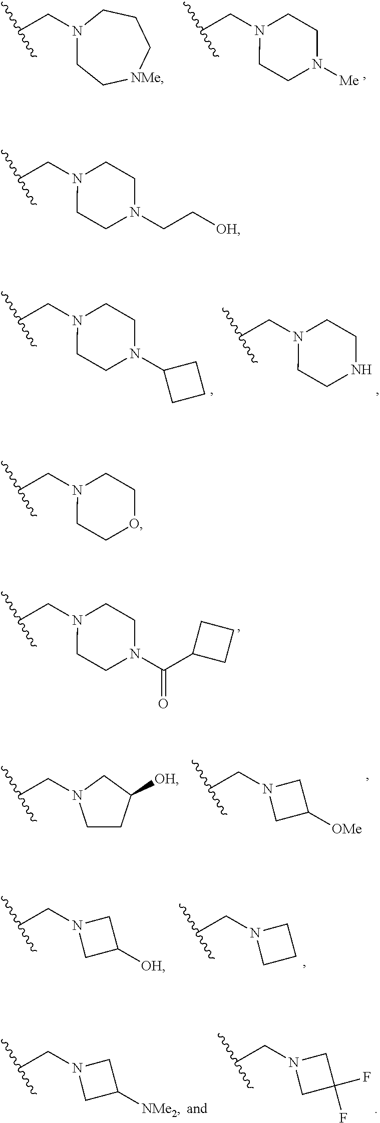

In one aspect, W is, preferably in combination with formula (I′), (Ia), (If), or (Ig),

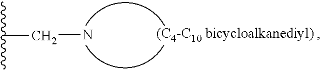 [SMILES: *CN]

with specific exemplary embodiments being

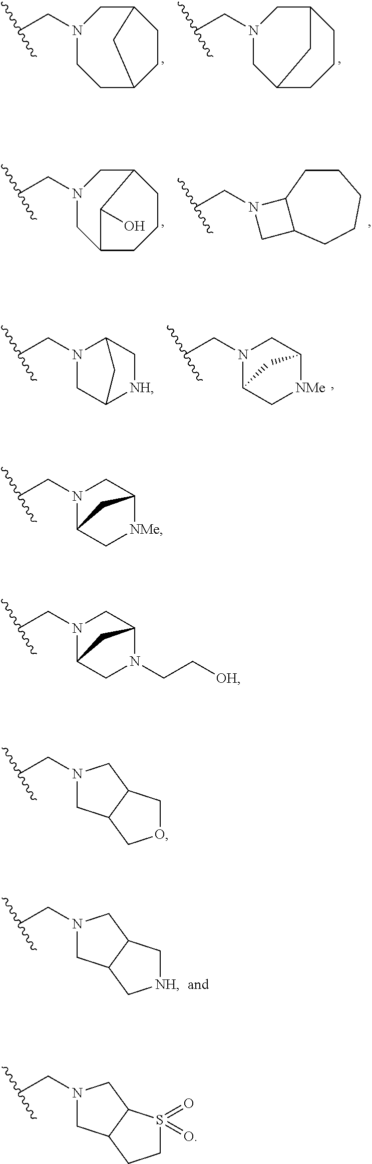

In one aspect, W is, preferably in combination with formula (I′), (Ia), (If), or (Ig),

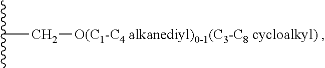

especially

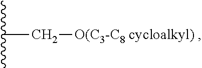

with a specific exemplary embodiment being

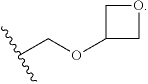 [SMILES: *COC1COC1]

In one aspect, W is, preferably in combination with formula (I′), (Ia), (If), or (Ig),

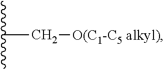

with specific exemplary embodiments being

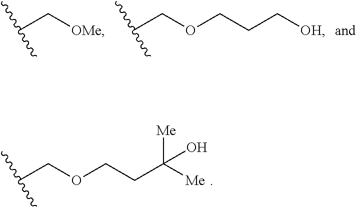

In one aspect, W is, preferably in combination with formula (I′), (Ia), (If), or (Ig),

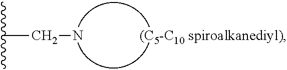 [SMILES: *CN]

with specific exemplary embodiments being

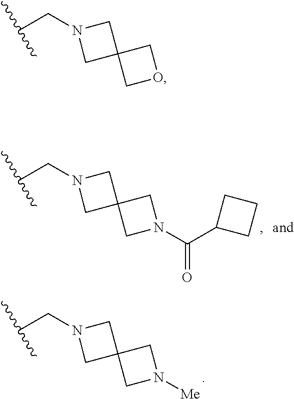 [SMILES: *CN1CC2(COC2)C1 ; *CN1CC2(C1)CN(C(=O)C1CCC1)C2 ; *CN1CC2(CN(C)C2)C1]

In one aspect, W is, preferably in combination with formula (I′), (Ia), (If), or (Ig), CH₂—N(C₁-C₅ alkyl){[C(═O)]_₁(C₁-C₅ alkyl)}

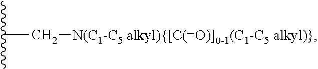

with specific exemplary embodiments being

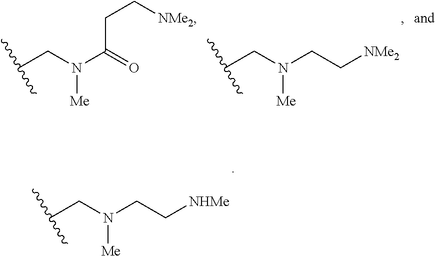 [SMILES: *CN(C)C(=O)CCN(C)C ; *CN(C)CCN(C)C ; *CN(C)CCNC]

In one aspect, W is, preferably in combination with formula (I′), (Ia), (If), or (Ig),

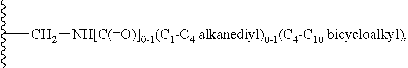

especially

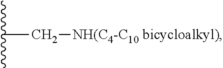

with a specific exemplary embodiment being

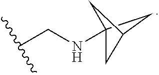 [SMILES: *CNC12CC(C1)C2]

In one aspect, W is, preferably in combination with formula (I′), (Ia), (If), or (Ig),

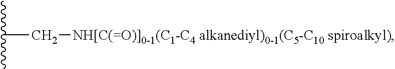

especially

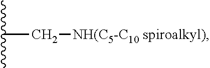

with specific exemplary embodiments being

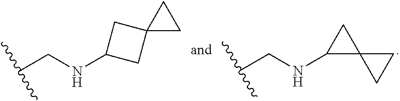 [SMILES: *CNC1CC2(CC2)C1 ; *CNC1CC12CC2]

In one aspect, W is, preferably in combination with formula (I′), (Ia), (If), or (Ig),

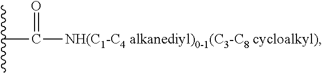

especially

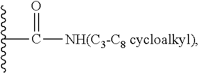

with specific exemplary embodiments being

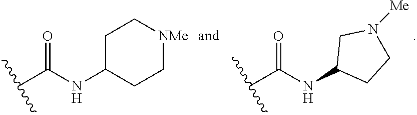

In one aspect, W is, preferably in combination with formula (I′), (Ia), (If), or (Ig),

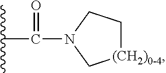

with a specific exemplary embodiment being

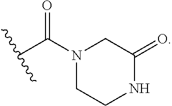 [SMILES: *C(=O)N1CCNC(=O)C1]

In one aspect, compounds of this disclosure are according to formula (If)

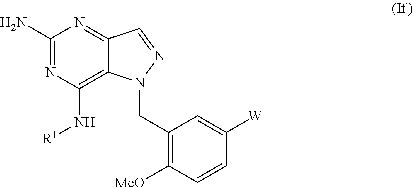

wherein
R¹ is

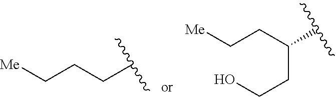 [SMILES: *CCCC ; *[C@@H](CCC)CCO]

and
W is

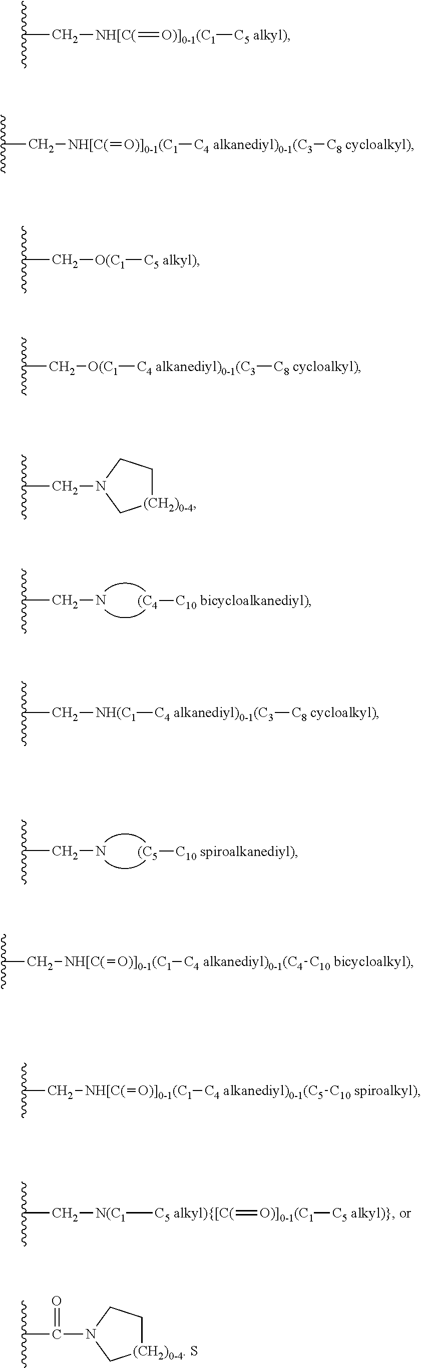

Some of the above exemplary alkyl, cycloalkyl, spiroalkyl, bicyloalkyl, etc., groups and moieties of the formula

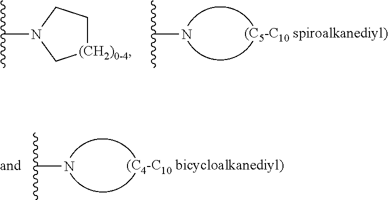

bear optional substituents and/or optionally have one or more CH₂ groups replaced by O, SO₂, etc., as described in the BRIEF SUMMARY OF THE DISCLOSURE above.

Specific examples of compounds disclosed herein per formula (Ia) are shown in the following Table A1. The table also provides data relating to biological activity: human TLR7 agonism reporter assay and/or induction of the CD69 gene in human whole blood, determined per the procedures provided hereinbelow. The right-most column contains analytical data (mass spectrum, LC/MS retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

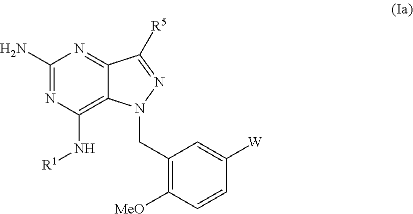

TABLE Al
Compounds According to Formula (Ia)

| | Structure | hTLR7 | hWB | Analytical Data (Mass Spectrum, Retention |
|---|---|---|---|---|
| Cpd. | (R⁵ = H unless noted | Agonism | CD69 | Time, ¹H-NMR (500 MHz, DMSO-d₆ unless |
| No. | otherwise) | EC₅₀ (nM) | EC₅₀ (nM) | noted otherwise) |
| 101 | 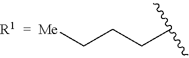 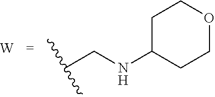 N7-butyl-1-[(2-methoxy-5- {[(oxan-4-yl)amino]- methyl}phenyl)methyl]-1H- pyrazolo[4,3-d]pyrimidine- 5,7-diamine | 351.8 | | LC/MS [M + H]⁺: 440.2 LC RT (min)/condition: 1.3/A δ 7.55 (s, 1H), 7.22 (dd, J = 8.3, 2.1 Hz, 1H), 6.99 (d, J = 8.4 Hz, 1H), 6.54 (d, J = 2.2 Hz, 1H), 6.35 (s, 1H), 5.63 (d, J = 14.1 Hz, 4H), 3.83 (s, 3H), 3.76 (d, J = 11.5 Hz, 2H), 3.41- 3.36 (m, 1H), 3.17-3.09 (m, 2H), 2.42- 2.34 (m, 1H), 1.91 (d, J = 1.9 Hz, 4H), 1.59 (d, J = 12.8 Hz, 2H), 1.46 (p, J = 7.2 Hz, 2H), 1.17 (dt, J = 16.3, 8.3 Hz, 4H), 0.84 (t, J = 7.4 Hz, 3H) |
| 102 | 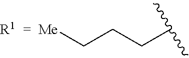 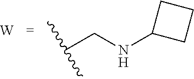 1-{[5-({3-azabicyclo- [4.2.1]nonan-3-yl}methyl)- 2-methoxyphenyl]methyl}- N7-butyl-1H-pyrazolo[4,3- d]pyrimidine-5,7-diamine | 658.4 | | LC/MS [M + H]⁺: 464.3 LC RT (min)/condition: 1.7/A δ 8.26 (s, 1H), 7.96 (s, 1H), 7.79 (s, 1H), 7.51 (d, J = 8.5 Hz, 1H), 7.15 (t, J = 9.4 Hz, 1H), 6.90 (s, 1H), 5.75 (d, J = 3.4 Hz, 2H), 4.32-4.18 (m, 2H), 3.82 (s, 3H), 3.59-3.52 (m, 1H), 3.30-3.16 (m, 1H), 3.01 (s, 1H), 2.82-2.72 (m, 2H), 2.34 (s, 2H), 2.36-2.29 (m, 2H), 2.10-1.99 (m, 2H), 1.82-1.18 (m, 9H), 0.89 (t, J = 7.3 Hz, 3H) |
| 103 | 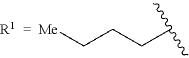 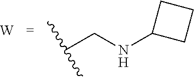 1-{[5-({3-azabicyclo- [4.2.1]nonan-3-yl}methyl)- 2-methoxyphenyl]methyl}- N7-butyl-1H-pyrazolo[4,3- d]pyrimidine-5,7-diamine | 193.0 | | LC/MS [M + H]⁺: 410.0 LC RT (min)/condition: 1.3/A δ 7.55 (t, J = 2.4 Hz, 1H), 7.22-7.16 (m, 1H), 7.01-6.94 (m, 1H), 6.52 (s, 1H), 6.36 (s, 1H), 5.64-5.58 (m, 2H), 3.82 (s, 3H), 3.56-3.45 (m, 1H), 3.41-3.36 (m, 1H), 2.98-2.91 (m, 1H), 1.95-1.85 (m, 3H), 1.60- 1.43 (m, 7H), 1.23-1.15 (m, 2H), 0.85 (tt, J = 7.0, 2.8 Hz, 3H) |
| 104 | 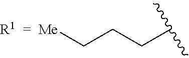 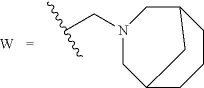 1-{[5-({3-azabicyclo- [3.3.1]nonan-3-yl}methyl)- 2-methoxyphenyl]methyl}- N7-butyl-1H-pyrazolo[4,3- d]pyrimidine-5,7-diamine | 630.1 | | LC/MS [M + H]⁺: 464.2 LC RT (min)/condition: 2.4/A δ 7.60 (s, 1H), 7.13 (d, J = 8.7 Hz, 1H), 7.00 (d, J = 8.3 Hz, 1H), 6.65 (s, 1H), 6.46 (d, J = 2.0 Hz, 1H), 6.14 (s, 1H), 5.67 (s, 2H), 3.85 (s, 3H), 3.14 (s, 1H), 2.64 (d, J = 10.8 Hz, 2H), 2.24-2.14 (m, 1H), 2.01 (d, J = 10.7 Hz, 2H), 1.67 (s, 2H), 1.53-1.12 (m, 14H), 0.82 (t, J = 7.4 Hz, 3H) |
| 105 | 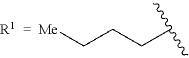 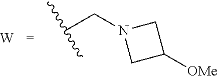 N7-butyl-1-({2-methoxy-5- [(3-methoxyazetidin-1- yl)methyl]phenyl}methyl)- 1H-pyrazolo[4,3- d]pyrimidine-5,7-diamine | 124.0 | 90.9 | LC/MS [M + H]⁺: 426.2 LC RT (min)/condition: 0.9/A δ 8.25 (s, 1H), 7.77 (s, 1H), 7.43 (d, J = 8.5 Hz, 1H), 7.10 (d, J = 8.5 Hz, 1H), 6.95 (s, 1H), 5.71 (s, 2H), 4.22 (d, J = 17.4 Hz, 3H), 4.14 (t, J = 8.6 Hz, 2H), 3.87-3.81 (m, 2H), 3.77 (s, 3H), 3.60-3.49 (m, 2H), 3.23 (s, 3H), 1.57 (p, J = 7.4 Hz, 2H), 1.26 (p, J = 7.4 Hz, 2H), 0.88 (t, J = 7.3 Hz, 3H) |
| 106 | 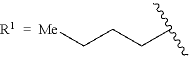 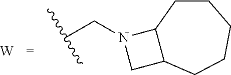 1-{[5-({8-azabicyclo- [5.2.0]nonan-8-yl}methyl)- 2-methoxyphenyl]methyl}- N7-butyl-1H-pyrazolo[4,3- d]pyrimidine-5,7-diamine | 501.7 | | LC/MS [M + H]⁺: 464.2 LC RT (min)/condition: 1.4/A δ 7.57 (s, 1H), 7.16-7.10 (m, 1H), 6.97 (d, J = 8.3 Hz, 1H), 6.48 (d, J = 2.0 Hz, 1H), 6.30 (s, 1H), 5.62 (s, 2H), 3.83 (s, 3H), 3.41-3.35 (m, 1H), 3.19-3.12 (m, 1H), 2.86-2.74 (m, 2H), 2.33-2.25 (m, 2H), 1.92 (s, 2H), 1.77- 1.03 (m, 14H), 0.85 (t, J = 7.4 Hz, 3H) |
| 107 | 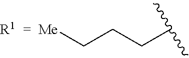 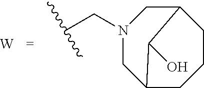 3-[(3-{[5-amino-7-(butyl- amino)-1H-pyrazolo[4,3- d]pyrimidin-1-yl]methyl}-4- methoxyphenyl)methyl]-3- aza bicyclo[3.3.1]nonan-9- ol | 749.8 | | LC/MS [M + H]⁺: 480.2 LC RT (min)/condition: 1.8/A δ 7.57 (s, 1H), 7.16-7.10 (m, 1H), 6.97 (d, J = 8.3 Hz, 1H), 6.48 (d, J = 2.0 Hz, 1H), 6.30 (s, 1H), 5.62 (s, 2H), 3.83 (s, 3H), 3.41-3.35 (m, 1H), 3.19-3.12 (m, 1H), 2.86-2.74 (m, 2H), 2.33-2.25 (m, 2H), 1.92 (s, 2H), 1.77- 1.03 (m, 14H), 0.85 (t, J = 7.4 Hz, 3H) |
| 108 | 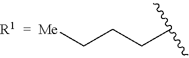 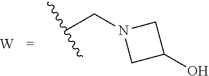 1-[(3-{[5-amino-7-(butyl- amino)-1H-pyrazolo[4,3- d]pyrimidin-1-yl]methyl}-4- methoxyphenyl)me- thyl]azetidin-3-ol | 146.0 | | LC/MS [M + H]⁺: 412.4 LC RT (min)/condition: 1.1/A δ 7.56 (s, 1H), 7.13 (d, J = 8.3 Hz, 1H), 6.98 (d, J = 8.4 Hz, 1H), 6.48 (d, J = 2.2 Hz, 1H), 6.39 (s, 1H), 5.62 (d, J = 17.0 Hz, 4H), 4.09 (t, J = 6.2 Hz, 1H), 3.82 (s, 3H), 3.39-3.30 (m, 2H), 2.62 (t, J = 6.9 Hz, 2H), 1.92 (s, 4H), 1.48 (p, J = 7.3 Hz, 2H), 1.20 (q, J = 7.4 Hz, 2H), 0.85 (t, J = 7.4 Hz, 3H) |
| 109 | 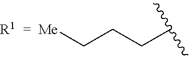 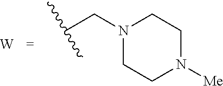 N7-butyl-1-({2-methoxy-5- [(4-methylpiperazin-1- yl)methyl]phenyl}methyl)- 1H-pyrazolo[4,3-d] pyrimidine-5,7-diamine | 51.3 | | LC/MS [M + H]⁺: 439.2 LC RT (min)/condition: 1.1/A δ 7.57 (s, 1H), 7.14 (dd, J = 8.3, 2.0 Hz, 1H), 6.99 (d, J = 8.5 Hz, 1H), 6.41 (d, J = 2.2 Hz, 1H), 6.35 (s, 1H), 5.63 (d, J = 8.8 Hz, 4H), 3.40-3.21 (m, 2H), 2.56 (s, 1H), 2.23- 2.17 (m, 4H), 2.14 (s, 3H), 1.92 (s, 1H), 1.44 (p, J = 7.3 Hz, 2H), 1.16 (h, J = 7.4 Hz, 2H), 0.83 (t, J = 7.3 Hz, 3H) |
| 110 | 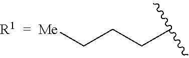 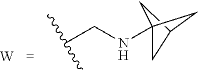 1-({5-[({bicyclo[1.1.1]- pentan-1-yl}amino) methyl]-2-methoxyphenyl}- methyl)-N7-butyl-1H- pyrazolo[4,3-d]pyrimidine- 5,7-diamine | 446.7 | | LC/MS [M + H]⁺: 422.2 LC RT (min)/condition: 1.6/A δ 8.26 (s, 1H), 7.87 (s, 1H), 7.76 (s, 1H), 7.46 (dd, J = 8.5, 2.4 Hz, 1H), 7.11 (d, J = 8.5 Hz, 1H), 7.02 (d, J = 2.2 Hz, 1H), 5.72 (s, 2H), 3.99 (s, 2H), 3.78 (s, 3H), 3.61-3.54 (m, 1H), 2.63 (s, 1H), 2.56 (s, 1H), 1.93 (s, 6H), 1.60 (h, J = 7.5, 6.8 Hz, 2H), 1.31 (dt, J = 14.8, 7.3 Hz, 2H), 1.17 (t, J = 7.3 Hz, 1H), 0.91 (t, J = 7.4 Hz, 3H) |
| 111 | 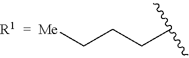 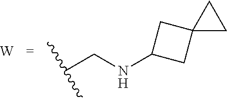 N7-butyl-1-({2-methoxy-5- [({spiro[2.3]hexan-5- yl}amino)methyl]phenyl} methyl)-1H-pyrazolo[4,3- d]pyrimidine-5,7-diamine | 357.7 | | LC/MS [M + H]⁺: 436.1 LC RT (min)/condition: 1.5/A δ 7.55 (s, 1H), 7.21 (d, J = 8.2 Hz, 1H), 6.99 (d, J = 8.4 Hz, 1H), 6.55 (d, J = 2.2 Hz, 1H), 6.33 (s, 1H), 5.60 (s, 2H), 3.82 (s, 3H), 3.44- 3.37 (m, 1H), 3.18 (p, J = 7.4 Hz, 1H), 2.55 (s, 2H), 1.92-1.85 (m, 5H), 1.47 (p, J = 7.2 Hz, 2H), 1.21 (dt, J = 14.9, 7.4 Hz, 2H), 0.85 (t, J = 7.3 Hz, 3H), 0.35 (dd, J = 9.0, 6.0 Hz, 2H), 0.28 (dd, J = 9.7, 6.0 Hz, 2H) |
| 112 | 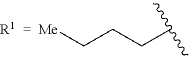 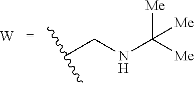 N7-butyl-1-({5-[(tert- butylamino)methyl]-2- methoxyphenyljmethyl)- 1H-pyrazolo[4,3- d]pyrimidine-5,7-diamine | 248.5 | | LC/MS [M + H]⁺: 412.9 LC RT (min)/condition: 1.2/A δ 7.36 (d, J = 8.5 Hz, 1H), 7.09 (d, J = 8.4 Hz, 1H), 6.86 (s, 1H), 6.53 (s, 1H), 5.63 (s, 2H), 3.82 (s, 3H), 3.78 (s, 1H), 3.44-3.37 (m, 1H), 3.18 (p, J = 7.4 Hz, 1H), 2.55 (s, 2H), 1.55-1.47 (m, 2H), 1.29-1.22 (m, 2H), 1.20 (s, 9H), 0.89 (t, J = 7.3 Hz, 3H) |
| 113 | 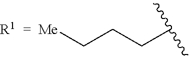 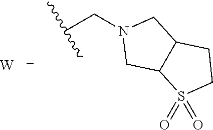 5-[(3-{[5-amino-7-(butyl- amino)-1H-pyrazolo[4,3- d]pyrimidin-1-yl]methyl}-4- methoxyphenyl)methyl]- hexahydro-2H-1λ6-thieno- [2,3-c]pyrrole-1,1-dione | 492.8 | | LC/MS [M + H]⁺: 499.2 LC RT (min)/condition: 1.6/A δ 7.58 (s, 1H), 7.14 (d, J = 8.4 Hz, 1H), 7.00 (d, J = 8.4 Hz, 1H), 6.37-6.28 (m, 2H), 5.73- 5.64 (m, 3H), 5.58 (d, J = 17.1 Hz, 1H), 3.85(s, 3H), 3.53-3.30 (m, 2H), 3.15 (d, J = 13.4 Hz, 1H), 3.09 (d, J = 10.5 Hz, 1H), 2.99 (d, J = 15.4 Hz, 2H), 2.66 (td, J = 12.5, 6.8 Hz, 2H), 2.55 (d, J = 1.1 Hz, 1H), 2.34 (dd, J = 11.7, 8.1 Hz, 2H), 2.13 (t, J = 8.3 Hz, 1H), 2.04 (tt, J = 13.4, 7.6 Hz, 1H), 1.65 (d, J = 10.4 Hz, 1H), 1.44 (p, J = 7.1 Hz, 2H), 1.14 (h, J = 7.4 Hz, 2H), 0.82 (t, J = 7.4 Hz, 3H) |
| 114 | 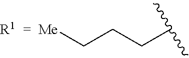 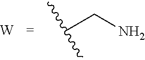 1-{[5-(aminomethyl)-2- methoxyphenyl]methyl}- N7-butyl-1H-pyrazolo[4,3- d]pyrimidine-5,7-diamine | 233.6 | 6.7 | LC/MS [M + H]⁺: 356.4 LC RT (min)/condition: 1.0/A δ 7.55 (s, 1H), 7.29 (d, J = 8.4 Hz, 1H), 7.02 (d, J = 8.4 Hz, 1H), 6.72 (s, 1H), 6.43 (s, 1H), 5.60 (d, J = 5.6 Hz, 4H), 3.82 (s, 3H), 3.61- 3.31 (m, 2H), 2.55 (s, 2H), 1.84 (s, 2H), 1.50 (t, J = 7.6 Hz, 2H), 1.23 (q, J = 7.6 Hz, 2H), 0.87 (t, J = 7.3 Hz, 3H) |
| 115 | 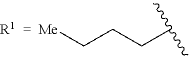 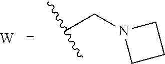 1-({5-[(azetidin-1-yl) methyl]-2-methoxyphenyl}- methyl)-N7-butyl-1H- pyrazolo[4,3-d]pyrimidine- 5,7-diamine | 171.5 | | LC/MS [M + H]⁺: 396.1 LC RT (min)/condition: 1.3/A δ 7.57 (s, 1H), 7.13 (d, J = 8.4 Hz, 1H), 6.97 (d, J = 8.4 Hz, 1H), 6.46 (d, J = 1.9 Hz, 1H), 6.39 (s, 1H), 5.62 (d, J = 13.2 Hz, 6H), 3.82 (s, 3H), 2.96-2.92 (m, 2H), 2.56 (s, 4H), 1.95-1.86 (m, 4H), 1.47 (q, J = 7.3 Hz, 2H), 1.26-1.15 (m, 2H), 0.85 (t, J = 7.3 Hz, 3H) |
| 116 | 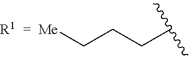 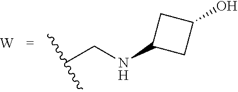 (1r,3r)-3-{[(3-{[5-amino-7- (butylamino)-1H-pyrazolo- [4,3-d]pyrimidin-1-yl] methyl}-4-methoxyphenyl)- methyl]amino}cyclobutan- 1-ol | 334.7 | 204.6 | LC/MS [M + H]⁺: 426.4 LC RT (min)/condition: 1.0/A δ 7.55 (s, 1H), 7.22 (d, J = 8.1 Hz, 1H), 6.99 (d, J = 8.4 Hz, 1H), 6.63 (d, J = 2.3 Hz, 1H), 6.41 (s, 1H), 5.61 (d, J = 10.5 Hz, 2H), 4.21 (t, J = 6.0 Hz, 1H), 3.81 (s, 3H), 3.41 (s, 1H), 3.16 (q, J = 9.6, 7.5 Hz, 1H), 2.56 (s, 1H), 1.92 (s, 5H), 1.92-1.82 (m, 2H), 1.50 (p, J = 7.3 Hz, 2H), 1.23 (h, J = 7.5 Hz, 2H), 0.87 (t, J = 7.4 Hz, 3H) |
| 117 | 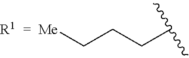 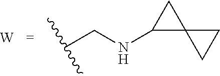 N7-butyl-1-({2-methoxy-5- [({spiro[2.2]pentan-1- yl}amino)methyl]phenyl} methyl)-1H-pyrazolo[4,3- d]pyrimidine-5,7-diamine | 561.8 | 156.4 | LC/MS [M + H]⁺: 421.9 LC RT (min)/condition: 1.4/A δ 7.73 (d, J = 2.5 Hz, 1H), 7.44 (dd, J = 8.6, 2.3 Hz, 1H), 7.10 (dd, J = 8.4, 5.1 Hz, 1H), 6.99-6.93 (m, 1H), 5.70 (d, J = 5.3 Hz, 2H), 4.11-4.01 (m, 2H), 3.90 (d, J = 2.8 Hz, 1H), 3.80 (s, 3H), 3.57-3.44 (m, 1H), 3.18 (s, 1H), 2.84-2.79 (m, 1H), 1.60-1,55 (m, 2H), 1.31- 1.24 (m, 2H), 1.21-1.08 (m, 2H), 0.89 (t, J = 7.3 Hz, 3H), 0.84-0.70 (m, 4H) |
| 118 | 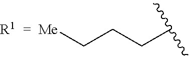 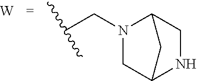 N7-butyl-1-{[5-({2,5-diaza- bicyclo[2.2.1]heptan-2- yl}methyl)-2-methoxy- phenyl]methyl}-1H- pyrazolo[4,3-d]pyrimidine- 5,7-diamine | 422.8 | 334.8 | LC/MS [M + H]⁺: 437.2 LC RT (min)/condition: 1.1/A δ 8.30 (d, J = 6.0 Hz, 1H), 7.75 (s, 1H), 7.43 (d, J = 8.1 Hz, 1H), 7.08 (d, J = 8.5 Hz, 2H), 5.69 (s, 2H), 4.37 (s, 1H), 4.15-4.05 (m, 2H), 3.75 (s, 3H), 3.63-3.50 (m, 2H), 3.29-3.08 (m, 2H), 3.00 (s, 1H), 2.40-2.27 (m, 2H), 1.98-1.88 (m, 2H), 1.59 (p, J = 7.3 Hz, 2H), 1.28 (q, J = 7.5 Hz, 2H), 0.90 (t, J = 7.3 Hz, 3H) |
| 119 | 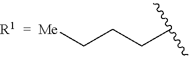 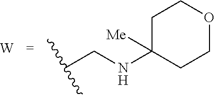 N7-butyl-1-[(2-methoxy-5- {[(4-methyloxan-4- yl)amino]methyl}phenyl) methyl]-1H-pyrazolo[4,3- d]pyrimidine-5,7-diamine | 529.1 | 227.2 | LC/MS [M + H]⁺: 454.2 LC RT (min)/condition: 1.1/A δ 7.54 (s, 1H), 7.27-7.21 (m, 1H), 6.97 (d, J = 8.4 Hz, 1H), 6.70-6.65 (m, 1H), 6.42 (t, J = 5.5 Hz, 1H), 5.58 (d, J = 6.5 Hz, 4H), 3.80 (s, 3H), 3.75-3.65 (m, 4H), 3.46-3.36 (m, 4H), 1.50-1.40 (m, 4H), 1.38-1.32 (m, 4H), 1.18 (p, J = 7.6 Hz, 2H), 1.03 (s, 3H), 0.84 (t, J = 7.4 Hz, 3H) |
| 120 | 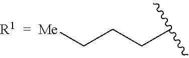 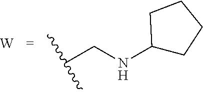 N7-butyl-1-({5-[(cyclo- pentylamino)methyl]-2- methoxyphenyljmethyl)- 1H-pyrazolo[4,3- d]pyrimidine-5,7-diamine | 548.4 | 288 | LC/MS [M + H]⁺: 424.4 LC RT (min)N7-butyl-1-({5-[(cyclo- pentylamino)methyl]-2- methoxyphenyljmethyl)- 1H-pyrazolo[4,3- d]pyrimidine-5,7-diamine/condition: 1.0/A δ 7.55 (d, J = 1.1 Hz, 1H), 7.25-7.19 (m, 1H), 6.98 (d, J = 8.4 Hz, 1H), 6.54 (s, 1H), 6.35 (t, J = 5.5 Hz, 1H), 5.59 (s, 2H), 3.81 (s, 3H), 3.61 (d, J = 5.3 Hz, 1H), 3.47 (s, 1H), 3.38 (q, J = 6.5 Hz, 2H), 1.54 (s, 4H), 1.45 (p, J = 7.2 Hz, 2H), 1.38 (s, 2H), 1.17 (dq, J = 14.5, 7.2 Hz, 4H), 0.86-0.79 (m, 3H) |
| 121 | 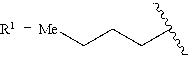 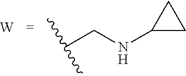 N7-butyl-1-({5-[(cyclo- propylamino)methyl]-2- methoxyphenyljmethyl)- 1H-pyrazolo[4,3-d] pyrimidine-5,7-diamine | 115.9 | 56.9 | LC/MS [M + H]⁺: 396.4 LC RT (min)/condition: 0.9/A δ 7.40 (s, 1H), 7.06 (dd, J = 8.4, 2.3 Hz, 1H), 6.84 (d, J = 8.4 Hz, 1H), 6.42 (d, J = 2.1 Hz, 1H), 6.25 (s, 1H), 5.46 (d, J = 9.9 Hz, 4H), 3.66 (s, 3H), 3.26-3.20 (m, 1H), 1.76 (s, 2H), 1.74-1.68 (m, 2H), 1.32 (p, J = 7.3 Hz, 2H), 1.05 (h, J = 7.4 Hz, 2H), 0.70 (t, J = 7.4 Hz, 3H), 0.08-0.01 (m, 4H) |
| 122 | 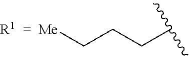 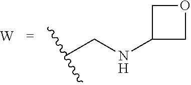 N7-butyl-1-[(2-methoxy-5- {[(oxetan-3-yl)amino]- methyl}phenyl)methyl]-1H- pyrazolo[4,3-d]pyrimidine- 5,7-diamine | 547.9 | 225.0 | LC/MS [M + H]⁺: 412.1 LC RT (min)/condition: 1.4/A δ 8.12 (s, 1H), 7.76 (s, 2H), 7.43 (dd, J = 8.6, 2.2 Hz, 1H), 7.10 (d, J = 8.5 Hz, 1H), 6.96 (d, J = 2.2 Hz, 1H), 5.71 (s, 2H), 4.57 (t, J = 7.4 Hz, 2H), 4.47 (t, J = 6.6 Hz, 2H), 4.25 (t, J = 6.6 Hz, 1H), 3.95 (s, 2H), 3.79 (s, 3H), 3.64- 3.55 (m, 2H), 1.59 (p, J = 7.4 Hz, 2H), 1.28 (dt, J = 15.0, 7.5 Hz, 2H), 0.90 (t, J = 7.4 Hz, 3H) |
| 123 | 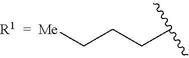 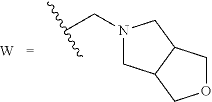 N7-butyl-1-{[5-({hexa- hydro-1H-furo[3,4-c]pyrrol- 5-yl}methyl)-2- methoxyphenyl]methyl}- 1H-pyrazolo[4,3-d] pyrimidine-5,7-diamine | 158.2 | 89.8 | LC/MS [M + H]⁺: 452.2. LC RT (min)/condition: 1.2/A δ 7.57 (s, 1H), 7.15 (dd, J = 8.2, 2.1 Hz, 1H), 6.99 (d, J = 8.4 Hz, 1H), 6.44-6.36 (m, 2H), 5.69 (s, 2H), 5.62 (s, 2H), 3.83 (s, 3H), 3.52- 3.18 (m, 6H), 2.60 (s, 2H), 2.37-2.33 (m, 2H), 2.10 (dd, J = 9.2, 3.0 Hz, 2H), 1.44 (p, J = 7.2 Hz, 2H), 1.16 (h,J = 7.3 Hz, 2H), 0.83 (t, J = 7.4 Hz, 3H) |
| 124 | 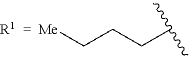 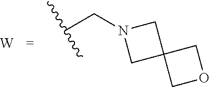 N7-butyl-1-{[2-methoxy-5- ({2-oxa-6-azaspiro- [3.3]heptan-6-yl}- methyl)phenyl]methyl}-1H- pyrazolo[4,3-d]pyrimidine- 5,7-diamine | 190.1 | 86.3 | LC/MS [M + H]⁺: 438.2 LC RT (min)/condition: 1.3/A δ 7.58 (s, 1H), 7.10 (dd, J = 8.4, 2.1 Hz, 1H), 6.97 (d, J = 8.5 Hz, 1H), 6.44 (s, 1H), 6.37 (d, J = 2.1 Hz, 1H), 5.67 (s, 1H), 5.61 (s, 2H), 4.53 (s, 4H), 3.82 (s, 3H), 3.39 (q, J = 6.5 Hz, 1H), 3.28 (s, 1H), 3.11 (s, 4H), 1.92 (s, 2H), 1.46 (p, J = 7.2 Hz, 2H), 1.17 (h, J = 7.4 Hz, 2H), 0.84 (t, J = 7.4 Hz, 3H) |
| 125 | 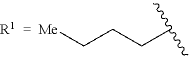 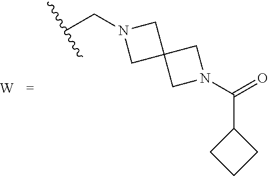 N7-butyl-1-{[5-({6- cyclobutanecarbonyl-2,6- diazaspiro[3.3]heptan-2- yl}methyl)-2-methoxy- phenyl]methyl}-1H- pyrazolo[4,3-d]pyrimidine- 5,7-diamine | 191.4 | 368.9 | LC/MS [M + H]⁺: 519.0 LC RT (min)/condition: 1.5/A δ 7.59 (s, 1H), 7.16-7.10 (m, 1H), 6.99 (d, J = 8.5 Hz, 1H), 6.51 (s, 1H), 6.40 (s, 1H), 5.76 (s, 1H), 5.62 (s, 2H), 4.03 (s, 2H), 3.83 (s, 3H), 3.10-3.00 (m, 4H), 2.56 (s, 4H), 2.12- 1.71 (m, 9H), 1.48 (p, J = 7.3 Hz, 2H), 1.19 (h, J = 7.6 Hz, 2H), 0.85 (t, J = 7.3 Hz, 3H) |
| 126 | 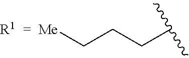 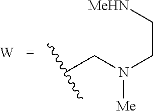 N7-butyl-1-{[2-methoxy-5- ({methyl[2-(methylamino)- ethyl]amino}methyl)phenyl ]methyl}-1H-pyrazolo[4,3- d]pyrimidine-5,7-diamine | 157.9 | 98.9 | LC/MS [M + H]⁺: 427.2 LC RT (min)/condition: 1.3/A δ 7.56 (s, 1H), 7.19 (dd, J = 8.5, 2.1 Hz, 1H), 7.00 (d, J = 8.4 Hz, 1H), 6.49 (d, J = 2.1 Hz, 1H), 6.40 (s, 1H), 5.63 (d, J = 6.4 Hz, 3H), 3.40 (q, J = 6.5 Hz, 2H), 3.28 (s, 3H), 2.64 (t, J = 6.3 Hz, 2H), 2.35-2.32 (m, 5H), 1.98 (s, 3H), 1.90 (s, 2H), 1.48 (p, J = 7.3 Hz, 2H), 1.21 (h, J = 7.5 Hz, 2H), 0.86 (t, J = 7.4 Hz, 3H) |
| 127 | 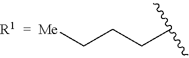 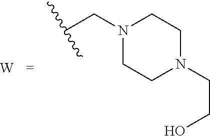 2-{4-[(3-{[5-amino-7- (butylamino)-1H-- pyrazolo[4,3-d]pyrimidin-1- yl]methyl}-4-methoxy- phenyl)methyl]piperazin-1- yl}ethan-1-ol | 107.0 | 47.5 | LC/MS [M + H]⁺: 469.2 LC RT (min)/condition: 1.2/A δ 8.29 (t, J = 5.8 Hz, 1H), 7.95 (s, 1H), 7.77 (s, 1H), 7.35-7.26 (m, 1H), 7.09-7.03 (m, 1H), 6.88 (s, 1H), 5.72 (s, 2H), 3.76 (s, 3H), 3.71 (t, J = 5.4 Hz, 2H), 3.59 (d, J = 6.6 Hz, 1H), 3.14-3.08 (m, 2H), 2.56-2.50 (m, 12H), 1.59 (p, J = 7.4 Hz, 2H), 1.28 (dt, J = 15.0, 7.5 Hz, 2H), 0.90 (t, J = 7.3 Hz, 3H) |
| 128 | 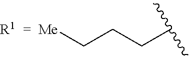 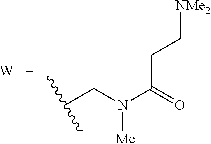 N-[(3-{[5-amino-7-(butyl- amino)-1H-pyrazolo[4,3- d]pyrimidin-1-yl]methyl}-4- methoxyphenyl)methyl]-3- (dimethylamino)-N- methylpropanamide | 56.2 | 12.6 | LC/MS [M + H]⁺: 469.2 LC RT (min)/condition: 1.2/A ¹H NMR (400 MHz, DMSO-d₆): δ 7.53 (s, 1H), 7.13-7.06 (m, 1H), 7.02 (dd, J = 22.1, 8.4 Hz, 1H), 6.35 (dd, J = 21.2, 4.4 Hz, 2H), 5.64-5.56 (m, 4H), 4.36 (s, 1.5H), 4.29 (s, 1.5H), 3.83 (d, J = 5.9 Hz, 3H), 2.67 (d, J = 21.1 Hz, 3H), 2.52-2.49 (m, 2H), 2.48- 2.26 (m, 4H), 2.15 (s, 3H), 2.04 (s, 2H), 1.51-1.42 (m, 2H), 1.19 (hept, J = 7.0 Hz, 2H), 0.84 (td, J = 7.3, 1.6 Hz, 3H) |
| 129 | 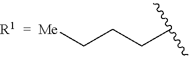 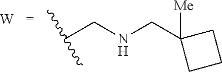 1-{[5-({[(1-aminocyclo- butyl)methyl]amino}methyl)- 2-methoxyphenyl]methyl}- N7-butyl-1H-pyrazolo[4,3- d]pyrimidine-5,7-diamine | 586.6 | 60.9 | LC/MS [M + H]⁺: 439.1 LC RT (min)/condition: 1.5/A δ 7.55 (s, 1H), 7.29 (dd, J = 25.1, 8.3 Hz, 1H), 7.00 (t, J = 8.6 Hz, 1H), 6.69 (s, 1H), 6.62 (d, J = 12.0 Hz, 1H), 5.60 (d, J = 9.0 Hz, 2H), 3.83 (s, 3H), 3.60-3.53 (m, 2H), 3.41- 3.36 (m, 2H), 1.95-1.53 (m, 6H), 1.48 (t, J = 8.1 Hz, 2H), 1.21 (dq, J = 14.8, 7.6, 7.2 Hz, 2H), 0.85 (q, J = 7.6 Hz, 3H) |
| 130 | 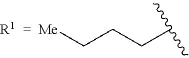 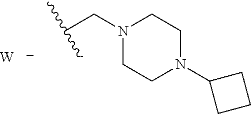 N7-butyl-1-({5-[(4- cyclobutylpiperazin-1- yl)methyl]-2-methoxy- phenyl}methyl)-1H- pyrazolo[4,3-d]pyrimidine- 5,7-diamine | 49.9 | 516.2 | LC/MS [M + H]⁺: 479.5 LC RT (min)/condition: 1.4/A δ 7.57 (s, 1H), 7.16-7.10 (m, 1H), 6.98 (d, J = 8.5 Hz, 1H), 6.40 (d, J = 2.1 Hz, 1H), 6.33 (s, 1H), 5.64 (d, J = 16.6 Hz, 3H), 3.83 (s, 3H), 3.39-3.34 (m, 1H), 3.22 (s, 1H), 2.64 (t, J = 7.8 Hz, 1H), 2.25-2.04 (m, 6H), 1.91 (s, 6H), 1.72 (t, J = 9.7 Hz, 2H), 1.64-1.57 (m, 2H), 1.44 (p, J = 7.2 Hz, 2H), 1.16 (h, J = 7.4 Hz, 2H), 0.83 (t, J = 7.4 Hz, 3H) |
| 131 | 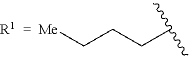 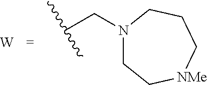 N7-butyl-1-({2-methoxy-5- [(4-methyl-1,4-diazepan-1- yl)methyl]phenyl}methyl)- 1H-pyrazolo[4,3-d] pyrimidine-5,7-diamine | 112.3 | 85.9 | LC/MS [M + H]⁺: 453.4 LC RT (min)/condition: 1.2/A δ 7.56 (s, 1H), 7.17-7.11 (m, 1H), 6.98 (d, J = 8.4 Hz, 1H), 6.46 (d, J = 2.3 Hz, 1H), 6.29 (s, 1H), 5.61 (d, J = 7.7 Hz, 3H), 3.83 (s, 3H), 3.38-3.34 (m, 2H), 2.47-2.49 (m, 2H), 2.43- 2.39 (m, 2H), 2.38-2.32 (m, 2H), 2.22 (s, 3H), 1.90 (s, 2H), 1.59-1.53 (m, 2H), 1.43 (p, J = 7.2 Hz, 2H), 1.15 (h, J = 7.4 Hz, 2H), 0.82 (t, J = 7.4 Hz, 3H) |
| 132 | 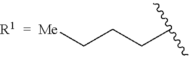 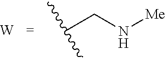 N7-butyl-1-({2-methoxy-5- [(methylamino)methyl] phenyl}methyl)-1H-pyrazolo- [4,3-d]pyrimidine-5,7- diamine | 186.5 | 10.9 | LC/MS [M + H]⁺: 370.3 LC RT (min)/condition: 1.3/A δ 7.55 (s, 1H), 7.26 (d, J = 8.3 Hz, 1H), 7.03 (d, J = 8.4 Hz, 1H), 6.65 (d, J = 2.1 Hz, 1H), 6.40 (s, 1H), 5.61 (d, J = 5.7 Hz, 3H), 3.83 (s, 3H), 3.56-3.19 (m, 2H), 2.22 (s, 3H), 1.90 (s, 2H), 1.48 (p, J = 7.2 Hz, 2H), 1.21 (h, J = 7.1 Hz, 2H), 0.86 (t, J = 7.3 Hz, 3H) |
| 133 | 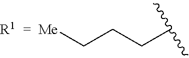 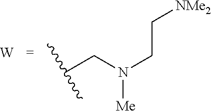 N7-butyl-1-{[5-({[2- (dimethylamino)ethyl](methyl)- amino}methyl)-2-methoxy- phenyl]methyl}-1H-pyrazolo [4,3-d]pyrimidine-5,7- diamine | 176.0 | 17 | LC/MS [M + H]⁺: 441.2 LC RT (min)/condition: 1.3/A δ 7.55 (s, 1H), 7.17 (dd, J = 8.4, 2.1 Hz, 1H), 7.00 (d, J = 8.4 Hz, 1H), 6.52 (d, J = 2.1 Hz, 1H), 6.36 (t, J = 5.6 Hz, 1H), 5.62 (d, J = 3.6 Hz, 3H), 3.84 (s, 3H), 2.31 (dd, J = 14.2, 5.5 Hz, 4H), 2.12 (s, 6H), 2.01 (s, 4H), 1.48 (p, J = 7.3 Hz, 2H), 1.20 (h,J = 7.4 Hz, 2H), 0.85 (t, J = 7.4 Hz, 3H) |
| 134 | 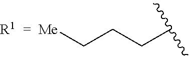 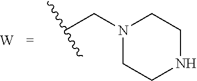 N7-butyl-1-({2-methoxy-5- [(piperazin-1-yl)methyl]- phenyl}methyl)-1H- pyrazolo[4,3-d]pyrimidine- 5,7-diamine | 201.0 | 62.5 | LC/MS [M + H]⁺: 425.1 LC RT (min)/condition: 1.1/A δ 8.29 (s, 1H), 7.75 (s, 1H), 7.36 (d, J = 8.0 Hz, 1H), 7.06 (d, J = 8.5 Hz, 1H), 6.97 (s, 1H), 5.70 (s, 2H), 3.81 (s, 1H), 3.75 (s, 2H), 3.68-3.56 (m, 2H), 3.22-3.15 (m, 2H), 2.89- 2.80 (m, 2H), 2.55 (s, 6H), 1.58 (p, J = 7.6 Hz, 2H), 1.27 (h, J = 7.5 Hz, 2H), 0.89 (t, J = 7.4 Hz, 3H) |
| 135 | 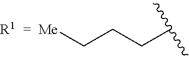 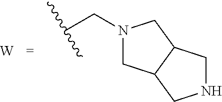 N7-butyl-1-{[2-methoxy-5- ({octahydropyrrolo[3,4-c]- pyrrol-2-yl}methyl)phenyl]- methyl}-1H-pyrazolo[4,3- d]pyrimidine-5,7-diamine | 448.2 | 325.2 | LC/MS [M + H]⁺: 449.1 LC RT (min)/condition: 1.4/A δ 7.57 (s, 1H), 7.14 (d, J = 8.3 Hz, 1H), 6.98 (d, J = 8.3 Hz, 1H), 6.41 (s, 1H), 6.35 (s, 1H), 5.62 (s, 2H), 3.83 (s, 3H), 3.40-3.35 (m, 1H), 3.27 (s, 1H), 3.30 (s, 1H), 2.86-2.78 (s, 1H), 2.49-2.37 (m, 4H), 2.30-2.24 (m 2H), 2.10 (d, J = 9.1 Hz, 2H), 1.82 (s, 2H), 1.45 (dq, J = 14.6, 6.8 Hz, 2H), 1.26-1.12 (m, 2H), 0.83 (t, J = 7.4 Hz, 3H) |
| 136 | 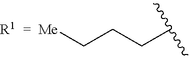 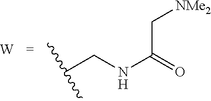 N-[(3-{[5-amino-7-(butyl- amino)-1H-pyrazolo[4,3- d]pyrimidin-1-yl]methyl}-4- methoxyphenyl)methyl]-2- (dimethylamino)acetamide | 51.1 | 19.1 | LC/MS [M + H]⁺: 441.1 LC RT (min)/condition: 1.2/A δ 8.96 (d, J = 5.5 Hz, 1H), 8.25 (s, 1H), 7.76- 7.72 (m, 1H), 7.23 (d, J = 8.5 Hz, 1H), 7.00 (d, J = 8.4 Hz, 1H), 6.87 (d, J = 2.2 Hz, 1H), 5.68 (s, 2H), 4.23 (d, J = 5.8 Hz, 2H), 3.88 (s, 3H), 3.72 (s, 2H), 3.68-3.56 (m, 2H), 2.79 (s, 6H), 1.59 (p, J = 7.3 Hz, 2H), 1.32-1.21 (m, 2H), 0.89 (t, J = 7.3 Hz, 3H) |
| 137 | 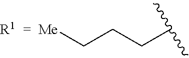 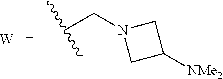 N7-butyl-1-[(5-{[3-(dime- thylamino)azetidin-1-yl]- methyl}-2-methoxyphenyl) methyl]-1H-pyrazolo- [4,3-d]pyrimidine-5,7- diamine | 114.2 | 28.1 | LC/MS [M + H]⁺: 439.2 LC RT (min)/condition: 1.1/A δ 7.57 (s, 1H), 7.13 (d, J = 10 Hz, 1H), 6.97 (d, J = 5 Hz, 1H), 6.44-6.36 (m, 2H), 5.62 (d, J = 7.5 Hz, 4H), 3.83 (s, 3H), 3.41-3.33 (m, 1H), 3.18-3.13 (m, 2H), 2.72-2.61 (m, 4H), 2.41 (s, 1H), 2.07(s, 1H), 1.96 (s, 6H), 1.47 (p, J = 7.1 Hz, 2H), 1.26-1.14 (m, 2H), 0.84 (t, J = 7.4 Hz, 3H) |
| 138 | 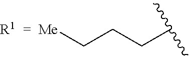 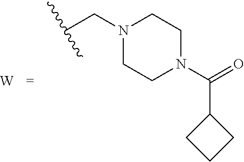 N7-butyl-1-({5-[(4-cyclo- butanecarbonylpiperazin-1- yl)methyl]-2-methoxy- phenyl}methyl)-1H-pyrazolo- [4,3-d]pyrimidine-5,7- diamine | 2067.5 | 1000 | LC/MS [M + H]⁺: 507.2 LC RT (min)/condition: 1.2/A δ 7.61 (s, 1H), 7.16 (dd, J = 8.5, 2.2 Hz, 1H), 7.00 (d, J = 8.4 Hz, 1H), 6.66 (s, 1H), 6.43 (d, J = 2.1 Hz, 1H), 5.99 (s, 1H), 5.65 (s, 2H), 3.82 (s, 3H), 3.30-3.25 (m, 2H), 3.19-3.14 (m, 2H), 2.15-2.02 (m, HH), 1.94-1.82 (m, 2H), 1.75-1.69 (m, 2H), 1.45 (p, J = 7.2 Hz, 2H), 1.16 (h, J = 7.4 Hz, 2H), 0.83 (t, J = 7.3 Hz, 3H) |
| 139 | 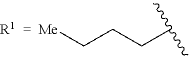 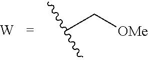 N7-butyl-1-{[2-methoxy-5- (methoxymethyl)phenyl]- methyl}-1H-pyrazolo[4,3- d]pyrimidine-5,7-diamine | 330.7 | 893.5 | LC/MS [M + H]⁺: 371.1 LC RT (min)/condition: 1.5/A δ 8.21 (s, 1H), 7.75 (s, 2H), 7.28-7.22 (m, 1H), 7.01 (d, J = 8.4 Hz, 1H), 6.85 (d, J = 2.1 Hz, 1H), 5.71 (s, 2H), 4.25 (s, 2H), 3.75 (s, 3H), 3.61 (s, 2H), 3.19 (s, 3H), 1.57 (p, J = 7.3 Hz, 2H), 1.27 (p, J = 7.4 Hz, 2H), 0.88 (t, J = 7.4 Hz, 3H) |
| 140 | 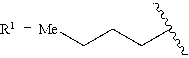 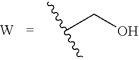 (3-{[5-amino-7-(butyl- amino)-1H-pyrazolo[4,3- d]pyrimidin-1-yl]methyl}-4- methoxyphenyl)methanol | 27.5 | 134.5 | LC/MS [M + H]⁺: 357.2 LC RT (min)/condition: 1.7/C ¹H NMR (400 MHz, DMSO-d₆) δ 7.55 (s, 1H), 7.20 (dd, J = 8.5, 2.2 Hz, 1H), 7.00 (d, J = 8.4 Hz, 1H), 6.63 (d, J = 2.1 Hz, 1H), 6.37 (t, J = 5.5 Hz, 1H), 5.61 (s, 4H), 4.99 (s, 1H), 4.28 (s, 2H), 3.83 (s, 3H), 3.40 (td, J = 7.0, 5.5 Hz, 2H), 1.56-1.43 (m, 2H), 1.23 (h, J = 7.4 Hz, 2H), 0.86 (t, J = 7.3 Hz, 3H) |
| 141 | 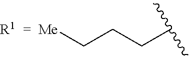 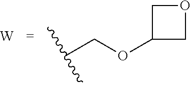 N7-butyl-1-({2-methoxy-5- [(oxetan-3-yloxy)me- thyl]phenyl}methyl)-1H- pyrazolo[4,3-d]pyrimidine- 5,7-diamine | 497.8 | 830.9 | LC/MS [M + H]⁺: 413.4 LC RT (min)/condition: 1.4/A δ 8.24 (s, 1H), 7.77 (s, 1H), 7.28 (dd, J = 8.2, 2.2 Hz, 1H), 7.02 (d, J = 8.4 Hz, 1H), 6.85 (d, J = 2.1 Hz, 1H), 5.72 (s, 2H), 4.58-4.47 (m, 4H), 4.33-4.22 (m, 4H), 3.76 (s, 3H), 3.63- 3.55 (m, 1H), 1.59 (p, J = 7.4 Hz, 2H), 1.28 (dt, J = 15.0, 7.5 Hz, 2H), 0.89 (t, J = 7.3 Hz, 3H) |
| 142 | 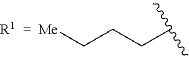 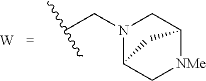 N7-butyl-1-[(2-methoxy-5- {[(1S,4S)-5-methyl-2,5- diazabicyclo[2.2.1]heptan-2- yl]methyl}phenyl)methyl]- 1H-pyrazolo[4,3-d] pyrimidine-5,7-diamine | 94.2 | 29.5 | LC/MS [M + H]⁺: 449.2 LC RT (min)/condition: 1.3/A δ 7.58 (s, 1H), 7.20-7.14 (m, 1H), 6.99 (d, J = 8.5 Hz, 1H), 6.44 (d, J = 2.1 Hz, 1H), 6.37 (s, 1H), 5.69-5.57 (m, 3H), 3.83 (s, 3H), 3.42-3.33 (m, 1H), 3.09 (s, 1H), 2.71 (d, J = 10.2 Hz, 1H), 2.50 (s, 1H), 2.41 (dd, J = 10.3, 2.5 Hz, 1H), 2.36 (s, 3H), 1.92 (s, 4H), 1.62 (q, J = 9.9 Hz, 2H), 1.45 (p, J = 7.1 Hz, 2H), 1.17 (h, J = 7.4 Hz, 2H), 0.84 (t, J = 7.3 Hz, 3H) |
| 143 | 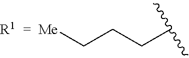 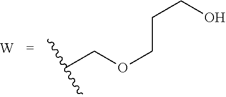 3-[(3-{[5-amino-7-(butyl- amino)-1H-pyrazolo[4,3- d]pyrimidin-1-yl]methyl}-4- methoxyphenyl)- methoxy]propan-1-ol | 424.4 | 724.6 | LC/MS [M + H]⁺: 415.1 LC RT (min)/condition: 1.5/A δ 7.57 (s, 1H), 7.21 (dd, J = 8.4, 2.3 Hz, 1H), 7.02 (d, J = 8.4 Hz, 1H), 6.58 (d, J = 2.2 Hz, 2H), 5.79 (s, 1H), 5.63 (s, 2H), 4.23 (s, 2H), 3.82 (s, 3H), 3.49-3.39 (m, 2H), 3.34 (t, J = 6.5 Hz, 2H), 2.56 (s, 1H), 1.92 (s, 1H), 1.58 (p, J = 6.5 Hz, 2H), 1.49 (p, J = 7.3 Hz, 2H), 1.21 (h, J = 7.3 Hz, 2H), 0.85 (t, J = 7.4 Hz, 3H) |
| 144 | 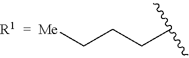 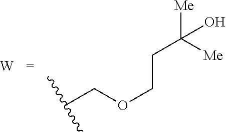 4-[(3-{[5-amino-7-(butyl- amino)-1H-pyrazolo[4,3- d]pyrimidin-1-yl]methyl}-4- methoxyphenyl)methoxy]- 2-methylbutan-2-ol | 562.3 | 1000.0 | LC/MS [M + H]⁺: 442.9 LC RT (min)/condition: 1.5/A δ 7.60 (s, 1H), 7.21 (dd, J = 8.4, 2.1 Hz, 1H), 7.01 (d, J = 8.3 Hz, 1H), 6.86 (s, 1H), 6.63 (d, J = 2.1 Hz, 1H), 5.63 (s, 2H), 4.23 (s, 2H), 3.81 (s, 3H), 3.45-3.37 (m, 4H), 1.53 (dt, J = 35.0, 7.3 Hz, 4H), 1.20 (h, J = 7.3 Hz, 2H), 1.03 (s, 6H), 0.84 (t, J = 7.4 Hz, 3H) |
| 145 | 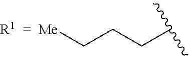 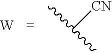 3-{[5-amino-7-(butyl- amino)-1H-pyrazolo[4,3- d]pyrimidin-1-yl]methyl}-4- methoxybenzonitrile | 402.1 | 942.2 | LC/MS [M + H]⁺: 410.2 LC RT (min)/condition: 1.5/D δ 7.79 (dd, J = 8.6, 2.2 Hz, 1H), 7.60 (s, 1H), 7.23 (d, J = 8.6 Hz, 1H), 6.85 (d, J = 2.1 Hz, 1H), 6.73 (t, J = 5.4 Hz, 1H), 5.75 (s, 2H), 5.67 (s, 2H), 3.90 (s, 3H), 3.43 (q, J = 6.6 Hz, 2H), 1.57-1.45 (m, 2H), 1.28-1.14 (m, 2H), 0.85 (t, J = 7.4 Hz, 3H) |
| 146 | 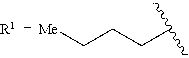 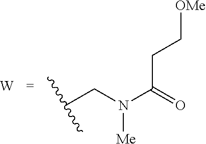 N-[(3-{[5-amino-7-(butyl- amino)-1H-pyrazolo[4,3- d]pyrimidin-1-yl]methyl}-4- methoxyphenyl)methyl]-3- methoxy-N- methylpropanamide | 499.1 | 1000.0 | LC/MS [M + H]⁺: 456.2 LC RT (min)/condition: 1.5/A δ 8.16 (s, 1H), 7.76 (s, 1H), 7.16 (d, J = 8.3 Hz, 1H), 7.02 (dd, J = 27.2, 8.3 Hz, 1H), 6.58 (s, 1H), 6.46 (s, 1H), 5.72 (d, J = 15.1 Hz, 2H), 4.42 (s, 1H), 4.34 (s, 1H), 3.76 (d, J = 9.0 Hz, 3H), 3.59-3.52 (m, 1H), 3.24 (s, 1H), 3.19 (s, 1H), 2.79 (s, 1H), 2.67 (s, 1H), 2.51 (dd, J = 3.7, 2.0 Hz, 6H), 2.45 (t, J = 6.6 Hz, 1H), 1.55 (s, 2H), 1.23 (q, J = 7.3 Hz, 2H), 0.87 (dd, J = 8.6, 6.4 Hz, 3H) |
| 147 | 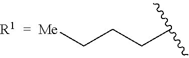 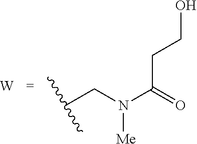 N-[(3-{[5-amino-7-(butyl- amino)-1H-pyrazolo[4,3- d]pyrimidin-1-yl]methyl}-4- methoxyphenyl)methyl]-3- hydroxy-N- methylpropanamide | 170.2 | 454.6 | LC/MS [M + H]⁺: 442.2 LC RT (min)/condition: 1.3/A δ 8.21 (d, J = 14.4 Hz, 1H), 7.82 (s, 1H), 7.76 (s, 1H), 7.17 (d, J = 7.8 Hz, 1H), 7.02 (dd, J = 27.3, 8.3 Hz, 1H), 6.60 (s, 1H), 5.72 (d, J = 14.1 Hz, 2H), 4.44 (s, 1H), 4.35 (s, 1H), 3.76 (d, J = 8.2 Hz, 3H), 3.68-3.44 (m, 3H), 2.93 (s, 0H), 2.80 (s, 2H), 2.67 (s, 1H), 2.42 (dt, J = 32.8, 6.6 Hz, 4H), 1.55 (q, J = 7.4 Hz, 2H), 1.27-1.15 (s, 2H), 0.87 (t, J = 7.4 Hz, 3H) |
| 148 | 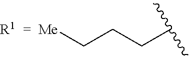 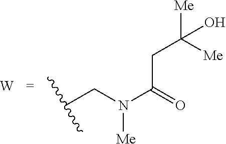 N-[(3-{[5-amino-7-(butyl- amino)-1H-pyrazolo[4,3- d]pyrimidin-1-yl]methyl}-4- methoxyphenyl)methyl]-3- hydroxy-N,3-dimethyl- butanamide | 665.4 | 1000.0 | LC/MS [M + H]⁺: 470.2 LC RT (min)/condition: 1.6/A δ 8.12 (s, 1H), 7.75 (d, J = 6.7 Hz, 1H), 7.17 (dd, J = 17.7, 8.4 Hz, 1H), 7.03 (dd, J = 26.5, 8.5 Hz, 1H), 5.72 (d, J = 20.0 Hz, 4H), 4.47 (s, 1H), 4.37 (s, 1H), 3.78 (d, J = 11.9 Hz, 3H), 3.59-3.41 (m, 2H), 2.82 (s, 2H), 2.68 (s, 1H), 2.55 (s, 4H), 2.40 (s, 1H), 2.30 (s, 1H), 1.54 (q, J = 7.7 Hz, 2H), 1.21 (dq, J = 18.6, 10.3, 9.0 Hz, 2H), 1.14 (s, 3H), 1.09 (s, 3H), 0.86 (q, J = 7.3 Hz, 3H) |
| 149 | 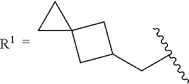 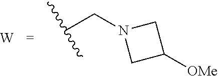 1-({2-methoxy-5-[(3- methoxyazetidin-1-yl)methyl]- phenyl}methyl)-N7-({spiro- [2.3]hexan-5-yl}methyl)- 1H-pyrazolo[4,3- d]pyrimidine-5,7-diamine | 271.4 | 525.4 | LC/MS [M + H]⁺: 464.0 LC RT (min)/condition: 1.3/A δ 7.35 (s, 1H), 6.93 (d, J = 7.9 Hz, 1H), 6.75 (d, J = 8.4 Hz, 1H), 6.38 (s, 1H), 6.24 (s, 1H), 5.63 (s, 1H), 5.38 (s, 2H), 3.68 (t, J = 5.8 Hz, 1H), 3.58 (s, 3H), 3.32 (t, J = 6.5 Hz, 1H), 2.89 (s, 3H), 2.63-2.56 (m, 2H), 2.44-2.37 (m, 2H), 1.76-1.68 (m, 4H), 1.54-1.49 (m, 3H), 0.70 (t, J = 7.5 Hz, 1H), 0.11 (s, 4H) |
| 150 | 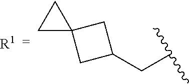 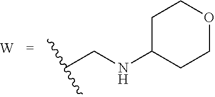 1-[(2-methoxy-5-{[(oxan-4- yl)amino]methyl}phenyl)- methyl]-N7-({spiro[2.3]- hexan-5-yl}methyl)-1H- pyrazolo[4,3-d]pyrimidine- 5,7-diamine | 387.4 | 298.2 | LC/MS [M + H]⁺: 478.1 LC RT (min)/condition: 1.2/A δ 8.33 (s, 1H), 7.90 (s, 1H), 7.77 (s, 1H), 7.51-7.45 (m, 1H), 7.12 (d, J = 8.5 Hz, 1H), 7.08-7.03 (m, 1H), 5.72 (s, 2H), 4.08 (s, 2H), 3.93 (d, J = 11.8 Hz, 2H), 3.77 (s, 3H), 3.47-3.39 (m, 1H), 3.30-3.15 (m, 2H), 2.83- 2.73 (m, 1H), 2.08 (t, J = 10.1 Hz, 2H), 1.98- 1.86 (m, 4H), 1.60-1.54 (m, 4H), 0.40 (s, 4H) |
| 151 | 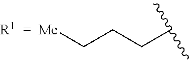 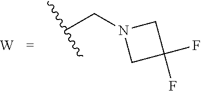 N7-butyl-1-({5-[(3,3-difluoro- azetidin-1-yl)methyl]-2- methoxyphenyl}methyl)- 1H-pyrazolo[4,3-d] pyrimidine-5,7-diamine | 767.6 | 1000.0 | LC/MS [M + H]⁺: 431.9 LC RT (min)/condition: 1.8/A δ 7.61-7.56 (m, 1H), 7.23-7.16 (m, 1H), 7.04-6.97 (m, 1H), 6.51 (s, 2H), 5.73 (s, 1H), 5.62 (d, J = 3.9 Hz, 2H), 3.82 (s, 3H), 3.59-3.48 (m, 2H), 3.46-3.37 (m, 4H), 2.59-2.54 (m, 2H), 1.50-1.46 (m, 2H), 1.23-1.16 (m, 2H), 0.85 (qd, J = 7.2, 6.1, 2.5 Hz, 3H) |
| 152 | 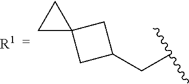 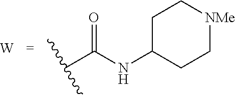 3-({5-amino-7-[({spiro- [2.3]hexan-5-yl}methyl)- amino]-1H-pyrazolo[4,3- d]pyrimidin-1-yl}methyl)-4- methoxy-N-(1- methylpiperidin-4- yl)benzamide | 467.2 | 158.8 | LC/MS [M + H]⁺: 505.1 LC RT (min)/condition: 1.2/A δ 7.81 (d, J = 7.6 Hz, 1H), 7.54 (d, J = 8.5 Hz, 1H), 7.29 (d, J = 2.3 Hz, 1H), 6.98 (d, J = 2.1 Hz, 1H), 6.80 (d, J = 8.7 Hz, 1H), 5.37 (d, J = 7.5 Hz, 3H), 3.59 (s, 3H), 2.52-2.48 (m, 2H), 1.90 (s, 3H), 1.75-1.62 (m, 8H), 1.54-1.40 (m, 4H), 1.32-1.26 (m, 2H), 0.08 (s, 4H) |
| 153 | 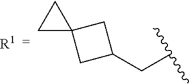 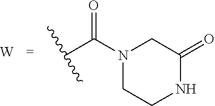 4-[3-({5-amino-7-[({spiro- [2.3]hexan-5-yl}methyl)- amino]-1H-pyrazolo[4,3- d]pyrimidin-1-yl}methyl)-4- methoxybenzoyl]piperazin- 2-one | 2245.3 | 484.8 | LC/MS [M + H]⁺: 410.0 LC RT (min)/condition: 1.5/A δ 8.35 (s, 1H), 8.11 (s, 1H), 7.83 (s, 1H), 7.76 (s, 1H), 7.44 (dd, J = 8.5, 2.1 Hz, 1H), 7.09 (d, J = 8.5 Hz, 1H), 6.94 (s, 1H), 5.74 (s, 2H), 3.95 (s, 1H), 3.79 (s, 3H), 3.75-3.71 (m, 2H), 3.56 (s, 5H), 3.21-3.16 (m, 1H), 2.79- 2.71 (m, 1H), 2.08-2.00 (m, 2H), 1.85 (dd, J = 11.7, 6.2 Hz, 2H), 0.36 (s, 4H) |
| 154 | 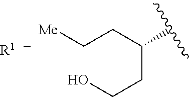 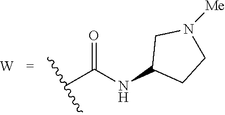 3-[(5-amino-7-{[(3S)-1- hydroxyhexan-3-yl]amino}- 1H-pyrazolo[4,3-d]pyrimidin- 1-yl)methyl]-4-methoxy- N-[(3R)-1-methylpyrrolidin- 3-yl] benzamide | 280.2 | 144.2 | LC/MS [M + H]⁺: 496 LC RT(min)/condition: 1.2/A δ 8.28-8.17 (m, 1H), 7.85-7.73 (m, 1H), 7.54-7.42 (m, 1H), 7.19-7.09 (m, 1H), 7.05-6.95 (m, 1H), 5.77-5.68 (m, 1H), 5.66-5.46 (m, 3H), 4.35-4.15 (m, 2H), 3.80 (s, 2H), 3.32-3.22 (m, 1H), 2.68-2.59 (m, 1H), 2.58-2.50 (m, 1H), 2.36 (br d, J = 6.1 Hz, 2H), 2.19 (s, 3H), 2.09-1.96 (m, 1H), 1.83 (s, 2H), 1.70-1.52 (m, 2H), 1.52- 1.41 (m, 1H), 1.39-1.24 (m, 2H), 1.13- 1.05 (m, 1H), 1.04-0.92 (m, 2H), 0.68 (s, 3H) |
| 155 | 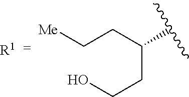 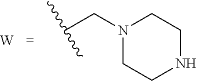 (3S)-3-{[5-amino-1-({2- methoxy-5-[(piperazin-1-yl)- methyl]phenyl}methyl)-1H- pyrazolo[4,3-d]pyrimidin-7- yl]amino}hexan-1-ol | 150.3 | 129.2 | LC/MS [M + H]⁺ 469.3 LC RT (min/condition) = 1.2/A δ 7.58 (s, 1H), 7.15 (d, J = 8.5 Hz, 1H), 7.01 (d, J = 8.5 Hz, 1H), 6.33 (s, 1H), 5.71-5.59 (m, 3H), 5.56 (d, J = 17.0 Hz, 1H), 4.30 (s, 1H), 3.84 (s, 3H), 3.31 (q, J = 7.6, 7.1 Hz, 1H), 3.19 (d, J = 12.4 Hz, 1H), 2.63 (d, J = 5.4 Hz, 4H), 2.55 (s, 2H), 2.13 (s, 4H), 1.88 (s, 2H), 1.61 (dt, J = 13.1, 6.5 Hz, 1H), 1.50 (dt, J = 13.7, 6.8 Hz, 1H), 1.35 (ddq, J = 36.8, 14.3, 7.9, 7.0 Hz, 1H), 1.01 (h, J = 7.4 Hz, 2H), 0.75 (t, J = 7.3 Hz, 3H) |
| 156 | 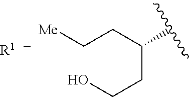 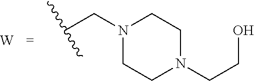 (3S)-3-({5-amino-1-[(5-{[4- (2-hydroxyethyl)piperazin- 1-yl]methyl}-2-methoxy- phenyl)methyl]-1H- pyrazolo[4,3-d]pyrimidin-7- yl}amino)hexan-1-ol | 56.9 | 73.6 | LC/MS [M + H]⁺ 513.0 LC RT (min/condition) = 1.1/A δ 7.59 (s, 1H), 7.15 (d, J = 8.8 Hz, 1H), 7.00 (d, J = 8.4 Hz, 1H), 6.33 (s, 1H), 5.69 (d, J = 16.4 Hz, 3H), 5.56 (d, J = 17.1 Hz, 1H), 4.29 (s, 1H), 3.84 (s, 3H), 3.32 (q, J = 7.2, 6.4 Hz, 1H), 3.22 (s, 1H), 2.55 (s, 4H), 2.41 (s, 2H), 2.37 (s, 4H), 2.19 (s, 4H), 1.91 (s, 2H), 1.62 (dd, J = 13.0, 6.5 Hz, 1H), 1.54-1.48 (m, 1H), 1.42-1.29 (m, 1H), 1.01 (q, J = 7.6 Hz, 2H), 0.75 (t, J = 7.2 Hz, 3H) |
| 157 | 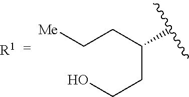 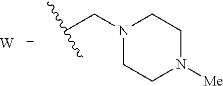 (3S)-3-{[5-amino-1-({2- methoxy-5-[(4-methyl- piperazin-1-yl)methyl]- phenyl}methyl)-1H- pyrazolo[4,3-d]pyrimidin-7- yl]amino}hexan-1-ol | 22.5 | 13.2 | LC/MS [M + H]⁺ 483.4 LC RT (min/condition) = 1.3/A δ 7.58 (s, 1H), 7.14 (d, J = 8.5 Hz, 1H), 7.00 (d, J = 8.4 Hz, 1H), 6.33 (s, 1H), 5.72-5.55 (m, 4H), 4.30 (s, 1H), 3.84 (s, 3H), 3.50 (s, 1H), 3.20 (s, 2H), 2.55 (s, 2H), 2.16 (s, 4H), 2.10 (s, 3H), 1.90 (s, 4H), 1.62 (dd, J = 13.3, 6.1 Hz, 1H), 1.54-1.47 (m, 1H), 1.35 (dq, J = 23.8, 7.4 Hz, 1H), 1.05-0.98 (m, 2H), 0.74 (t, J = 7.4 Hz, 3H) |
| 158 | 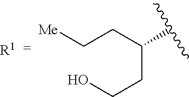 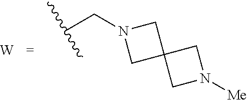 (3S)-3-[(5-amino-1-{[2- methoxy-5-({6-methyl-2,6- diazaspiro[3.3]heptan-2- yl}methyl)phenyl]methyl}- 1H-pyrazolo[4,3- d]pyrimidin-7-yl)amino]- hexan-1-ol | 603.6 | 198.9 | LC/MS [M + H]⁺ 495.2 LC RT (min/condition) = 1.3/A δ 7.60-7.56 (m, 2H), 7.13 (s, 1H), 7.00 (d, J = 8.8 Hz, 1H), 6.37 (s, 1H), 5.69 (d, J = 17.1 Hz, 2H), 5.56 (d, J = 16.5 Hz, 1H), 4.31 (s, 1H), 3.84 (s, 3H), 3.34 (s, 4H), 3.06 (s, 1H), 3.00 (s, 4H), 2.56 (s, 3H), 2.46 (s, 1H), 1.92 (s, 1H), 1.63 (s, 2H), 1.52 (s, 1H), 1.40 (s, 1H), 1.33 (s, 1H), 1.01 (d, J = 12.8 Hz, 2H), 0.76-0.73 (m, 3H) |
| 159 | 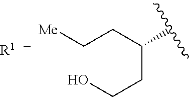 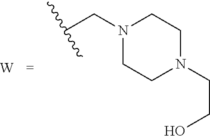 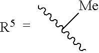 (3S)-3-({5-amino-1-[(5-{[4- (2-hydroxyethyl)piperazin- 1-yl]methyl}-2- methoxyphenyl)methyl]-3- methyl-1H-pyrazolo[4,3- d]pyrimidin-7- yl}amino)hexan-1-ol | 98.6 | 37.1 | LC/MS [M + H]⁺ 527.2 LC RT (min/condition) = 1.3 δ 7.17-7.11 (m, 1H), 7.00 (d, J = 8.3 Hz, 1H), 6.38 (d, J = 1.8 Hz, 1H), 5.61 (d, J = 17.9 Hz, 3H), 5.48 (d, J = 17.1 Hz, 1H), 4.31 (s, 1H), 3.91 (s, 1H), 3.84, (s, 3H), 3.32 (d, J = 6.5 Hz, 1H), 3.20 (d, J = 16.1 Hz, 1H), 2.56 (s, 3H), 2.34 (t, J = 6.3 Hz, 2H), 2.27 (s, 3H), 2.17 (s, 4H), 2.08 (s, 1H), 1.92 (s, 4H), 1.66- 1.59 (m, 1H), 1.50 (d, J = 5.8 Hz, 1H), 1.43- 1.30 (m, 1H), 1.25 (s, 1H), 1.03 (q, J = 7.5 Hz, 2H), 0.76 (t, J = 7.3 Hz, 3H) |
| 160 | 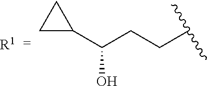 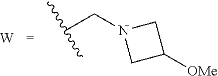 (1S)-3-{[5-amino-1-({2- methoxy-5-[(3-methoxy- azetidin-1-yl)methyl]phenyl}- methyl)-1H-pyrazolo[4,3- d]pyrimidin-7-yl]amino}-1- cyclopropylpropan-1-ol | 740.4 | 150.6 | LC/MS [M + H]⁺ = 468.13 RT (min) = 1.01 (LC/MS Procedure A) 1H NMR (500 MHz, DMSO-d6) δ 7.39 (s, 1H), 7.07-6.94 (m, 1H), 6.84-6.73 (m, 1H), 6.40-6.29 (m, 1H), 5.81-5.68 (m, 1H), 5.39 (s, 2H), 3.78-3.67 (m, 1H), 3.62 (s, 2H), 3.38-3.28 (m, 1H), 2.93 (s, 2H), 2.77-2.58 (m, 2H), 2.30 (br s, 6H), 1.71 (s, 3H), 1.62-1.51 (m, 1H), 1.48-1.36 (m, 1H), 0.64-0.51 (m, 1H), 0.20-0.07 (m, 2H), 0.04-−0.05 (m, 1H), −0.10-−0.19 (m, 1H) |
| 161 | 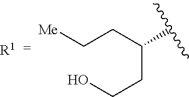 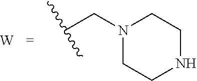 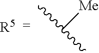 (3S)-3-{[5-amino-1-({2- methoxy-5-[(piperazin-1- yl)methyl]phenyl}methyl)- 3-methyl-1H-pyrazolo[4,3- d]pyrimidin-7- yl]amino}hexan-1-ol | 339.7 | 132.5 | LC/MS [M + H]⁺ 483.2 LC RT (min/condition) = 1.2/A δ 7.18-7.08 (m, 1H), 6.99 (dd, J = 11.3, 8.4 Hz, 1H), 6.35 (d, J = 2.1 Hz, 1H), 5.66-5.55 (m, 3H), 5.48 (d, J = 17.0 Hz, 1H), 4.30 (s, 1H), 3.84 (s, 3H), 3.32 (s, 2H), 3.23 (s, 1H), 2.74 (d, J = 6.9 Hz, 3H), 2.55 (s, 2H), 2.26 (d, J = 1.7 Hz, 3H), 2.22 (s, 4H), 1.90 (s, 4H), 1.66-1.58 (m, 1H), 1.49 (dd, J = 8.5, 5.5 Hz, 1H), 1.34 (ddt, J = 28.5, 14.2, 7.6 Hz, 1H), 1.02 (q, J = 7.4 Hz, 2H), 0.73 (dt, J = 14.5, 7.3 Hz, 3H) |
| 162 | 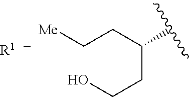 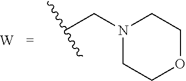 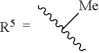 (3S)-3-({5-amino-1-[(2- methoxy-5-{[(oxan-4-yl) amino]methyl}phenyl)methyl]- 3-methyl-1H-pyrazolo[4,3- d]pyrimidin-7-yl}amino)- hexan-1-ol | 293.9 | 90.3 | LC/MS [M + H]⁺ 498.2 LC RT (min/condition) = 1.2/A δ 7.39 (d, J = 8.3 Hz, 1H), 7.12 (d, J = 8.5 Hz, 1H), 6.71 (s, 1H), 5.70 (d, J = 15.4 Hz, 2H), 5.60 (d, J = 17.0 Hz, 1H), 5.50 (d, J = 17.0 Hz, 1H), 4.33 (s, 1H), 3.86 (t, J = 13.1 Hz, 3H), 3.33 (q, J = 6.8, 5.9 Hz, 1H), 3.18 (t, J = 11.5 Hz, 2H), 2.89 (s, 1H), 2.73 (d, J = 0.7 Hz, 1H), 2.55 (s, 5H), 2.27 (d, J = 12.5 Hz, 1H), 2.26 (s, 3H), 1.91 (s, 2H), 1.79 (s, 2H), 1.64 (dt, J = 12.6, 6.6 Hz, 1H), 1.56-1.46 (m, 1H), 1.47-1.29 (m, 1H), 1.08 (dt, J = 15.1, 7.3 Hz, 2H), 0.78 (t, J = 7.4 Hz, 3H) |
| 163 | 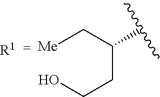 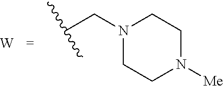 (3S)-3-{[5-amino-1-({2- methoxy-5-[(4-methylpiperazin- 1-yl)methyl]phenyl} methyl)-1H-pyrazolo[43-d] pyrimidin-7-yl]amino}pentan- l-ol | 267.2 | 119.1 | LC/MS [M + H]+ = 469.2 LC RT (min/condition) = 0.98/A δ 7.93 (s, 1H), 7.80 (s, 1H), 7.59 (d, J = 8.2 Hz, 1H), 7.28 (d, J = 7.8 Hz, 1H), 7.06 (t, J = 7.3 Hz, 1H), 6.72 (s, 1H), 5.83-5.67 (m, 2H), 4.44 (s, 1H), 3.78 (s, 3H), 3.03-2.85 (m, 1H), 2.74 (s, 3H), 2.56 (s, 6H), 1.83- 1.46 (m, 5H), 1.18 (t, J = 7.3 Hz, 1H), 0.77 (t, J = 7.4 Hz, 3H) |
| 164 | 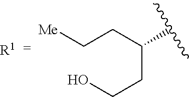 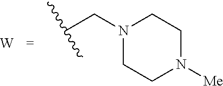 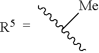 (3S)-3-{[5-amino-1-({2- methoxy-5-[(4-methylpiperazin- 1-yl)methyl]phenyl} methyl)-3-methyl-1H- pyrazolo[4,3-d]pyrimidin-7- yl]amino}hexan-1-ol | 48.5 | 21.7 | LC/MS [M + H]⁺ 497.2 LC RT (min/condition) = 1.3/A δ 7.95 (s, 1H), 7.18-7.12 (m, 1H), 7.00 (d, J = 8.4 Hz, 1H), 6.36 (s, 1H), 5.88 (s, 1H), 5.62 (d, J = 17.0 Hz, 1H), 5.50 (d, J = 17.1 Hz, 1H), 4.32 (d, J = 6.5 Hz, 1H), 3.83 (s, 3H), 3.32 (dd, J = 13.8, 7.9 Hz, 1H), 2.89 (s, 1H), 2.73 (d, J = 0.7 Hz, 1H), 2.55 (s, 2H), 2.42 (s, 3H), 2.27 (s, 7H), 1.91 (s, 4H), 1.62 (dt, J = 12.2, 6.5 Hz, 1H), 1.52 (dt, J = 13.8, 7.1 Hz, 1H), 1.43-1.30 (m, 1H), 1.02 (q, J = 7.5 Hz, 2H), 0.75 (t, J = 7.3 Hz, 3H) |
| 179 | 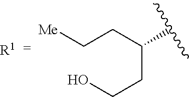 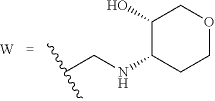 (3S,4S)-4-[({3-[(5-amino-7- {[(3S)-1-hydroxyhexan-3- yl]amino}-1H-pyrazolo[4,3- d]pyrimidin-1-yl)methyl]-4- methoxyphenyl}methyl)- amino]oxan-3-ol | 405.3 | | LC/MS [M + H]⁺ = 500.2 LC RT (min) = 0.97 (LC/MS Procedure A) δ 7.82-7.75 (m, 1H), 7.61-7.43 (m, 2H), 7.16-7.07 (m, 1H), 6.92 (s, 1H), 5.84-5.63 (m, 2H), 4.62-4.46 (m, 1H), 4.07-3.93 (m, 2H), 3.80 (s, 6H), 3.54-3.38 (m, 2H), 3.36- 3.29 (m, 1H), 3.27-3.18 (m, 1H), 3.17- 3.07 (m, 1H), 2.63 (s, 1H), 1.95-1.79 (m, 1H), 1.79-1.67 (m, 2H), 1.64 (br d, J = 10.7 Hz, 1H), 1.57-1.46 (m, 2H), 1.22-1.07 (m, 3H), 0.85-0.84 (m, 1H), 0.83 (br t, J = 7.3 Hz, 3H) |
| 180 | 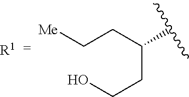 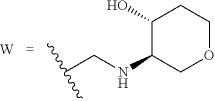 (3R,4R)-3-[({3-[(5-amino-7- {[(3S)-1-hydroxyhexan-3- yl]amino}-1H-pyrazolo[4,3- d]pyrimidin-1-yl)methyl]-4- methoxyphenyl}methyl)- amino]oxan-4-ol | 212.6 | | LC/MS [M + H]⁺ = 500.2 RT (min) = 1.00 (LC/MS Procedure A) δ 7.62-7.51 (m, 1H), 7.31-7.21 (m, 1H), 7.01 (d, J = 8.5 Hz, 1H), 6.67-6.54 (m, 1H), 5.73-5.49 (m, 5H), 4.41-4.27 (m, 1H), 3.83 (s, 3H), 3.75-3.64 (m, 2H), 3.48 (br s, 2H), 3.34 (br d, J = 6.1 Hz, 2H), 3.29-3.10 (m, 3H), 2.80 (br t, J = 10.4 Hz, 2H), 2.24 (td, J = 8.9, 4.4 Hz, 2H), 1.90 (s, 2H), 1.73 (br d, J = 9.5 Hz, 2H), 1.68-1.59 (m, 2H), 1.58- 1.47 (m, 2H), 1.47-1.29 (m, 6H), 1.13- 0.97 (m, 4H), 0.78 (br t, J = 7.3 Hz, 5H) |
| 181 | 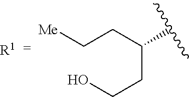 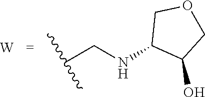 (3S,4R)-4-[({3-[(5-amino-7- {[(3S)-1-hydroxyhexan-3- yl]amino}-1H-pyrazolo[4,3- d]pyrimidin-1-yl)methyl]-4- methoxyphenyl}methyl)- amino]oxolan-3-ol | 307.6 | | LC/MS [M + H]⁺ = 486.2 RT (min) = 0.98 (LC/MS Procedure A) δ 7.54 (br d, J = 12.5 Hz, 1H), 7.28 (br d, J = 7.6 Hz, 1H), 7.04 (d, J = 8.2 Hz, 1H), 6.65 (s, 1H), 5.79 (br s, 3H), 5.70-5.50 (m, 3H), 4.35 (br s, 1H), 3.99 (br s, 1H), 3.83 (s, 3H), 3.77 (dd, J = 8.9, 5.2 Hz, 2H), 3.51-3.39 (m, 1H), 3.37-3.25 (m, 2H), 2.98 (br s, 1H), 1.92 (s, 1H), 1.71-1.58 (m, 2H), 1.52 (br d, J = 5.8 Hz, 2H), 1.48-1.29 (m, 3H), 1.15- 0.94 (m, 2H), 0.78 (br t, J = 7.3 Hz, 3H) |
| 182 | 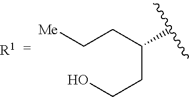 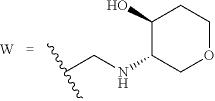 (3S,4S)-3-[({3-[(5-amino-7- {[(3S)-1-hydroxyhexan-3- yl]amino}-1H-pyrazolo[4,3- d]pyrimidin-1-yl)methyl]-4- methoxyphenyl}methyl)- amino]oxan-4-ol | 197.2 | | LC/MS [M + H]⁺ = 500.2 RT (min) = 1.01 (LC/MS Procedure A) δ 7.61-7.50 (m, 1H), 7.24 (br d, J = 8.2 Hz, 1H), 7.01 (d, J = 8.5 Hz, 1H), 6.60 (s, 1H), 5.75-5.48 (m, 6H), 4.33 (br s, 1H), 3.83 (s, 3H), 3.74-3.63 (m, 2H), 3.57-3.44 (m, 2H), 3.41-3.32 (m, 2H), 3.31-3.15 (m, 2H), 2.83-2.72 (m, 1H), 2.31-2.18 (m, 2H), 1.80-1.70 (m, 1H), 1.70-1.59 (m, 2H), 1.59-1.47 (m, 2H), 1.47-1.38 (m, 2H), 1.38-1.28 (m, 2H), 1.13-0.98 (m, 3H), 0.77 (br t, J = 7.3 Hz, 4H) |
| 183 | 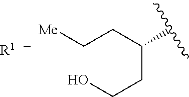 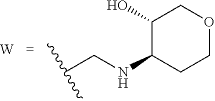 (3S,4R)-4-[({3-[(5-amino-7- {[(3S)-1-hydroxyhexan-3- yl]amino}-1H-pyrazolo[4,3- d]pyrimidin-1-yl)methyl]-4- methoxyphenyl}methyl)- amino]oxan-3-ol | 478.6 | | LC/MS [M + H]⁺ = 500.2 RT (min) = 1.01 (LC/MS Procedure A) δ 7.86-7.73 (m, 1H), 7.87-7.72 (m, 1H), 7.55-7.46 (m, 1H), 7.54-7.44 (m, 1H), 7.38-7.37 (m, 1H), 7.21-7.09 (m, 1H), 7.14 (d, J = 8.6 Hz, 1H), 6.93 (d, J = 1.6 Hz, 1H), 6.99-6.85 (m, 1H), 5.83-5.63 (m, 2H), 4.61-4.42 (m, 1H), 4.11 (s, 2H), 3.81 (s, 4H), 3.65-3.50 (m, 1H), 3.22-3.10 (m, 1H), 3.00-2.87 (m, 3H), 2.85-2.74 (m, 1H), 2.02-1.90 (m, 1H), 1.80-1.64 (m, 2H), 1.61-1.44 (m, 3H), 1.17 (t, J = 7.3 Hz, 6H), 0.91-0.73 (m, 3H) |
| 184 | 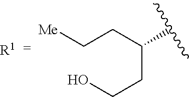 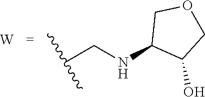 (3R,4S)-4-[({3-[(5-amino-7- {[(3S)-1-hydroxyhexan-3- yl]amino}-1H-pyrazolo[4,3- d]pyrimidin-1-yl)methyl]-4- methoxyphenyljmethyl)- amino]oxolan-3-ol | 338.8 | | LC/MS [M + H]⁺ = 486.2 RT (min) = 0.98 (LC/MS Procedure A) δ 7.61-7.51 (m, 1H), 7.24 (br d, J = 8.5 Hz, 1H), 7.01 (d, J = 8.5 Hz, 1H), 6.60 (s, 1H), 5.63 (br d, J = 3.7 Hz, 4H), 4.33 (br s, 1H), 3.98-3.87 (m, 2H), 3.83 (s, 3H), 3.74 (br d, J = 3.4 Hz, 2H), 3.50 (br s, 1H), 3.46-3.39 (m, 1H), 3.39-3.28 (m, 3H), 2.90 (br s, 2H), 1.91 (s, 1H), 1.77-1.57 (m, 2H), 1.57-1.47 (m, 2H), 1.47-1.39 (m, 2H), 1.39-1.17 (m, 2H), 1.15-0.94 (m, 2H), 0.77 (br t, J = 7.3 Hz, 3H) |

Additional compounds of this disclosure are shown in Table A2, along with their biological properties and analytical data.

TABLE A2
Additional Compounds of the Disclosure

| | | | hWB | Mass spectrum; LC/MS Retention |
|---|---|---|---|---|
| | | hTLR7 | CD69 | Time (Procedure A unless noted |
| Cpd | | Agonism | EC₅₀ | otherwise); ¹H NMR (500 MHz, DMSO- |
| No. | Structure | EC₅₀ (nM) | (nM) | d6 unless noted otherwise)) |
| 165 | 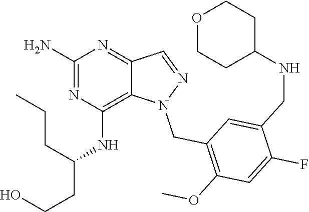 [SMILES: CCC[C@@H](CCO)Nc1nc(N)nc2cnn(Cc3cc(CNC4CCOCC4)c(F)cc3OC)c12] (3S)-3-({5-amino-1-[(4-fluoro-2- methoxy-5-{[(oxan-4-yl)amino]- methyl}phenyl)methyl]-1H- pyrazolo[4,3-d]pyrimidin-7- yl}amino)hexan-1-ol | 89.9 | 27.3 | LC/MS [M + H]⁺ 502.2 LC RT (min/condition) = 1.3/A δ 7.56 (s, 1H), 6.92 (d, J = 11.8 Hz, 1H), 6.54 (br d, J = 8.2 Hz, 1H), 5.68-5.58 (m, 4H), 5.52-5.47 (m, 1H), 4.34- 4.26 (m, 1H), 3.83 (s, 3H), 3.74-3.68 (m, 4H), 3.36-3.32 (m, 2H), 3.05 (br t, J = 11.7 Hz, 2H), 2.26-2.18 (m, 1H), 1.68-1.60 (m, 1H), 1.56-1.48 (m, 2H), 1.47-1.37 (m, 2H), 1.36-1.26 (m, 1H), 1.13-1.04 (m, 2H), 1.04- 0.95 (m, 2H), 0.74 (t, J = 7.3 Hz, 3H) |
| 166 | 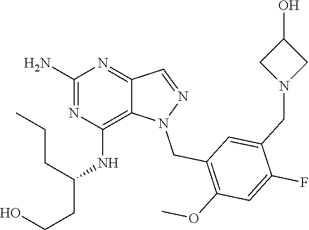 [SMILES: CCC[C@@H](CCO)Nc1nc(N)nc2cnn(Cc3cc(CN4CC(O)C4)c(F)cc3OC)c12] 1-({5-[(5-amino-7-{[(3S)-1- hydroxyhexan-3-yl]amino}-1H- pyrazolo[4,3-d]pyrimidin-1-yl) methyl]-2-fluoro-4- methoxyphenyl}- methyl)azetidin-3-ol | 116.3 | 34.2 | LC/MS [M + H]⁺ 474.2 LC RT (min/condition) = 1.23/A δ 7.57 (s, 1H), 6.92 (d, J = 12.0 Hz, 1H), 6.42 (d, J = 8.4 Hz, 1H), 5.75 (br d, J = 7.9 Hz, 1H), 5.66-5.57 (m, 3H), 5.52- 5.46 (m, 1H), 4.32 (br dd, J = 8.2, 4.2 Hz, 1H), 4.03 (quin, J = 6.2 Hz, 1H), 3.81 (s, 3H), 3.37-3.30 (m, 4H), 3.29-3.25 (m, 1H), 3.24-3.20 (m, 1H), 2.61- 2.55 (m, 2H), 1.68-1.60 (m, 1H), 1.58- 1.50 (m, 1H), 1.46-1.37 (m, 1H), 1.38-1.29 (m, 1H), 1.06-0.97 (m, 2H), 0.75 (t, J = 7.3 Hz, 3H) |
| 167 | 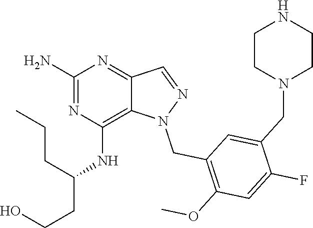 [SMILES: CCC[C@@H](CCO)Nc1nc(N)nc2cnn(Cc3cc(CN4CCNCC4)c(F)cc3OC)c12] (3S)-3-{[5-amino-1-({4-fluoro-2- methoxy-5-[(piperazin-1- yl)methyl]phenyl}methyl)-1H- pyrazolo[4,3-d]pyrimidin-7- yl]amino}hexan-1-ol | 189.4 | 145.0 | LC/MS [M + H]⁺ 487.2 LC RT (min/condition) = 1.27A δ 7.58 (s, 1H), 6.92 (d, J = 12.0 Hz, 1H), 6.27 (br d, J = 8.5 Hz, 1H), 5.78-5.73 (m, 2H), 5.67-5.61 (m, 1H), 5.59 (s, 2H), 5.55-5.48 (m, 1H), 4.32-4.25 (m, 1H), 3.83 (s, 3H), 3.35-3.27 (m, 2H), 3.23 (s, 2H), 2.62-2.56 (m, 4H), 2.11 (br s, 4H), 1.68-1.58 (m, 1H), 1.56-1.48 (m, 1H), 1.45-1.28 (m, 2H), 1.05-0.95 (m, 2H), 0.74 (br t, J = 7.3 Hz, 3H) |
| 168 | 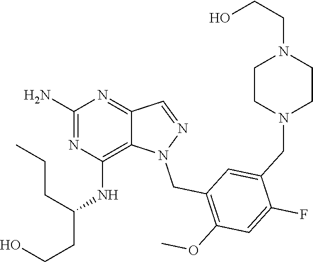 [SMILES: CCC[C@@H](CCO)Nc1nc(N)nc2cnn(Cc3cc(CN4CCN(CCO)CC4)c(F)cc3OC)c12] (3S)-3-({5-amino-1-[(4-fluoro-5-{[4- (2-hydroxyethyl)piperazin-1- yl]methyl}-2-methoxyphenyl)- methyl]-1H-pyrazolo[4,3- d]pyrimidin-7-yl}amino)hexan-1-ol | 62.8 | 39.2 | LC/MS [M + H]⁺ 531.3 LC RT (min/condition) = 1.30/A δ 7.58 (s, 1H), 6.91 (d, J = 11.7 Hz, 1H), 6.25 (br d, J = 8.3 Hz, 1H), 5.78 (br d, J = 8.6 Hz, 1H), 5.70-5.64 (m, 1H), 5.61 (s, 2H), 5.54-5.47 (m, 1H), 4.28 (br dd, J = 7.4, 5.0 Hz, 1H), 3.83 (s, 3H), 3.47-3.43 (m, 2H), 3.36-3.30 (m, 2H), 3.24 (s, 2H), 2.34 (br t, J = 6.4 Hz, 2H), 2.31-2.07 (m, 8H), 1.68-1.59 (m, 1H), 1.57-1.49 (m, 1H), 1.43- 1.27 (m, 2H), 0.99 (sxt, J = 7.4 Hz, 2H), 0.73 (t, J = 7.2 Hz, 3H) |
| 169 | 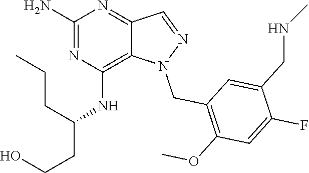 [SMILES: CCC[C@@H](CCO)Nc1nc(N)nc2cnn(Cc3cc(CNC)c(F)cc3OC)c12] (3S)-3-{[5-amino-1-({4-fluoro-2- methoxy-5-[(methylamino)- methyl]phenyl}methyl)-1H- pyrazolo[4,3-d]pyrimidin-7- yl]amino}hexan-1-ol | 43.5 | 6.2 | LC/MS [M + H]⁺ 432.0 LC RT (min/condition) = 1.18 δ 7.55 (s, 1H), 6.95 (d, J = 12.0 Hz, 1H), 6.64 (d, J = 8.4 Hz, 1H), 5.74 (br d, J = 7.9 Hz, 1H), 5.64-5.58 (m, 3H), 5.53- 5.48 (m, 1H), 4.37-4.29 (m, 1H), 3.82 (s, 3H), 3.37-3.32 (m, 2H), 2.12 (s, 3H), 1.70-1.61 (m, 1H), 1.59-1.49 (m, 1H), 1.47-1.31 (m, 2H), 1.10 (s, 2H), 0.80-0.73 (m, 3H) |
| 170 | 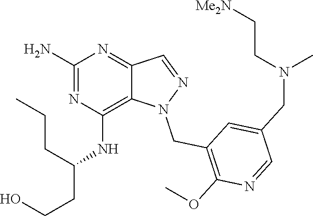 [SMILES: CCC[C@@H](CCO)Nc1nc(N)nc2cnn(Cc3cc(CN(C)CCN(C)C)cnc3OC)c12] (3S)-3-[(5-amino-1-{[5-({[2-(dimethyl- amino)ethyl](methyl)amino}- methyl)-2-methoxypyridin-3- yl]methyl}-1H-pyrazolo[4,3-d] pyrimidin-7-yl)amino]hexan-1-ol | 769.5 | 170.2 | LC/MS [M + H]⁺ 486.2 LC RT (min/condition) = 1.2/A δ 8.09-7.96 (m, 1H), 7.72-7.58 (m, 1H), 6.81-6.64 (m, 1H), 5.78-5.66 (m, 1H), 5.66-5.52 (m, 1H), 4.46- 4.30 (m, 1H), 3.96-3.86 (m, 2H), 3.46- 3.34 (m, 2H), 3.12-3.03 (m, 1H), 2.81-2.54 (m, 6H), 2.52-2.38 (m, 3H), 2.07-1.97 (m, 2H), 1.74-1.59 (m, 2H), 1.53-1.38 (m, 2H), 1.17- 0.99 (m, 2H), 0.84-0.70 (m, 3H) |
| 171 | 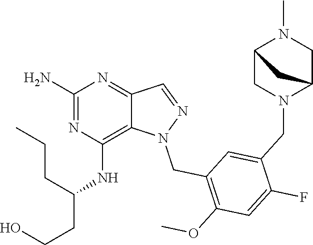 [SMILES: CCC[C@@H](CCO)Nc1nc(N)nc2cnn(Cc3cc(CN4C[C@H]5C[C@@H]4CN5C)c(F)cc3OC)c12] (3S)-3-({5-amino-1-[(4-fluoro-2- methoxy-5-{[(1R,4R)-5-methyl-2,5- diazabicyclo[2.2.1]heptan-2- yl]methyl}phenyl)methyl]-1H- pyrazolo[4,3-d]pyrimidin-7- yl}amino)hexan-1-ol | 436.8 | 139.8 | LC/MS [M + H]⁺ 513.3 LC RT (min/condition) = 1.24/A δ 7.59 (s, 1H), 6.93 (br d, J = 12.3 Hz, 1H), 6.33 (br d, J = 8.5 Hz, 1H), 5.84 (br d, J = 8.0 Hz, 1H), 5.73-5.62 (m, 3H), 5.59-5.52 (m, 1H), 4.35-4.26 (m, 1H), 3.89-3.84 (m, 2H), 3.84 (s, 3H), 3.13-3.10 (m, 1H), 2.59-2.55 (m, 2H), 1.95-1.91 (m, 1H), 1.81-1.69 (m, 2H), 1.68-1.61 (m, 1H), 1.59- 1.50 (m, 1H), 1.46-1.33 (m, 2H), 1.07- 0.99 (m, 2H), 0.78-0.73 (m, 3H) |
| 172 | 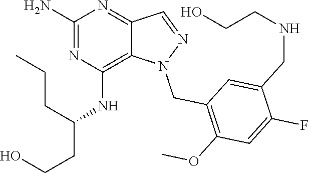 (3S)-3-({5-amino-1-[(4-fluoro-5-{[(2- hydroxyethyl)amino]methyl}-2- methoxyphenyl)methyl]-1H- pyrazolo[4,3-d]pyrimidin-7- yl}amino)hexan-1-ol | 167.2 | 29.8 | LC/MS [M + H]⁺ 426.2 LC RT (min/condition) = 1.14/A δ 7.57 (s, 1H), 7.02 (d, J = 11.7 Hz, 1H), 6.73 (d, J = 8.4 Hz, 1H), 5.81 (br d, J = 8.3 Hz, 1H), 5.68 (br s, 2H), 5.63 (br d, J = 16.6 Hz, 1H), 5.56-5.50 (m, 1H), 4.93-4.68 (m, 2H), 4.40-4.29 (m, 1H), 3.84 (s, 3H), 3.79-3.70 (m, 2H), 2.61 (br t, J = 5.2 Hz, 2H), 1.72-1.62 (m, 1H), 1.60-1.50 (m, 1H), 1.49- 1.32 (m, 2H), 1.08 (dq, J = 15.0, 7.3 Hz, 2H), 0.78 (t, J = 7.2 Hz, 3H) |
| 173 | 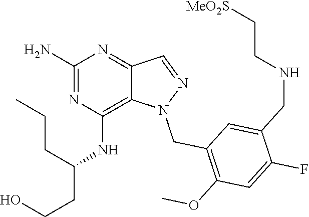 [SMILES: CCC[C@@H](CCO)Nc1nc(N)nc2cnn(Cc3cc(CNCCS(C)(=O)=O)c(F)cc3OC)c12] (3S)-3-({5-amino-1-[(4-fluoro-5-{[(2- methanesulfonylethyl)amino]methyl}- 2-methoxyphenyl)methyl]-1H- pyrazolo[4,3-d]pyrimidin-7- yl}amino)hexan-1-ol | 40.0 | 23.2 | LC/MS [M + H]⁺ 524.1 LC RT (min/condition) = 1.40/A δ 7.56 (s, 1H), 6.95 (br d, J = 12.1 Hz, 1H), 6.62 (br d, J = 8.3 Hz, 1H), 5.74 (br d, J = 7.7 Hz, 1H), 5.66-5.59 (m, 3H), 5.55-5.49 (m, 1H), 4.38-4.29 (m, 1H), 3.83 (s, 3H), 3.12 (br t, J = 6.2 Hz, 2H), 2.90 (s, 3H), 2.77 (br t, J = 6.3 Hz, 2H), 1.69-1.61 (m, 1H), 1.58-1.50 (m, 1H), 1.48-1.32 (m, 2H), 1.12- 1.02 (m, 2H), 0.78 (br t, J = 7.2 Hz, 3H) |
| 174 | 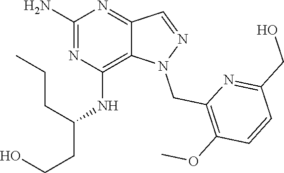 [SMILES: CCC[C@@H](CCO)Nc1nc(N)nc2cnn(Cc3nc(CO)ccc3OC)c12] (3S)-3-[(5-amino-1-{[6-(hydroxyl- methyl)-3-methoxypyridin-2- yl]methyl}-1H-pyrazolo[4,3- d]pyrimidin-7-yl)amino]hexan-1-ol | 96.1 | 41.4 | LC/MS [M + H]⁺ 402.3 LC RT (min/condition) = 1.08/E 1H NMR (400 MHz, DMSO-d6) δ = 7.56 (d, J = 8.6 Hz, 1H), 7.47 (s, 1H), 7.43 (d, J = 8.6 Hz, 1H), 7.13 (br d, J = 8.8 Hz, 1H), 5.68-5.57 (m, 4H), 5.35 (br d, J = 2.0 Hz, 1H), 4.53-4.48 (m, 1H), 4.44 (d, J = 5.6 Hz, 2H), 4.40- 4.34 (m, 1H), 3.84 (s, 3H), 3.48-3.46 (m, 2H), 3.17 (d, J = 4.6 Hz, 1H), 1.79- 1.66 (m, 2H), 1.61-1.51 (m, 2H), 1.35- 1.26 (m, 2H), 0.86 (t, J = 7.3 Hz, 3H) |
| 175 | 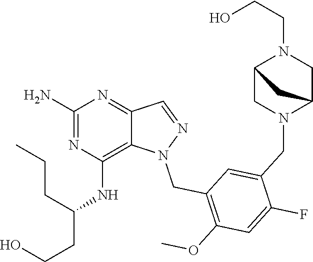 [SMILES: CCC[C@@H](CCO)Nc1nc(N)nc2cnn(Cc3cc(CN4C[C@H]5C[C@@H]4CN5CCO)c(F)cc3OC)c12] (3S)-3-({5-amino-1-[(4-fluoro-5- {[(1R,4R)-5-(2-hydroxyethyl)-2,5- diazabicyclo[2.2.1]heptan-2- yl]methyl}-2-methoxyphenyl)- methyl]-1H-pyrazolo[4,3- d]pyrimidin-7-yl}amino)hexan-1-ol | 203.8 | 77.0 | LC/MS [M + H]⁺ 543.0 LC RT (min/condition) = 1.24/A δ 7.57 (s, 1H), 6.90 (d, J = 12.0 Hz, 1H), 6.41 (br d, J = 8.3 Hz, 1H), 5.77 (br d, J = 8.1 Hz, 1H), 5.68-5.63 (m, 1H), 5.61 (s, 2H), 5.55-5.49 (m, 1H), 4.34-4.24 (m, 1H), 3.83 (s, 3H), 3.42-3.31 (m, 2H), 3.24 (br s, 1H), 2.93 (br s, 1H), 2.46-2.40 (m, 2H), 2.27-2.21 (m, 1H), 1.68-1.59 (m, 1H), 1.58-1.51 (m, 1H), 1.51-1.42 (m, 2H), 1.41- 1.29 (m, 2H), 1.01 (dq, J = 15.1, 7.7 Hz, 2H), 0.74 (t, J = 7.3 Hz, 3H) |
| 176 | 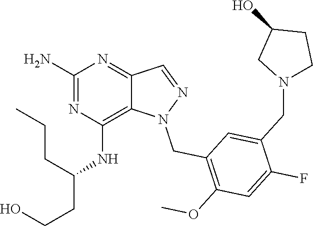 [SMILES: CCC[C@@H](CCO)Nc1nc(N)nc2cnn(Cc3cc(CN4CC[C@H](O)C4)c(F)cc3OC)c12] (3S)-1-({5-[(5-amino-7-{[(3S)-1- hydroxyhexan-3-yl]amino}-1H- pyrazolo[4,3-d]pyrimidin-1- yl)methyl]-2-fluoro-4- methoxyphenyl}methyl) pyrrolidin-3-ol | 57.2 | 5.7 | LC/MS [M + H]⁺ 488.2 LC RT (min/condition) = 1.22/A δ 7.56 (s, 1H), 6.92 (d, J = 11.9 Hz, 1H), 6.43 (d, J = 8.3 Hz, 1H), 5.80 (d, J = 8.2 Hz, 1H), 5.67-5.59 (m, 3H), 5.53- 5.47 (m, 1H), 4.71-4.59 (m, 1H), 4.35- 4.29 (m, 1H), 4.10-4.04 (m, 1H), 3.82 (s, 3H), 3.38-3.34 (m, 2H), 2.58- 2.55 (m, 1H), 2.28 (q, J = 7.7 Hz, 1H), 2.21-2.15 (m, 1H), 2.11 (dd, J = 9.5, 3.9 Hz, 1H), 1.88-1.80 (m, 1H), 1.70- 1.61 (m, 1H), 1.60 -1.51 (m, 1H), 1.46- 1.32 (m, 3H), 1.07-0.99 (m, 2H), 0.79-0.72 (m, 3H) |
| 177 | 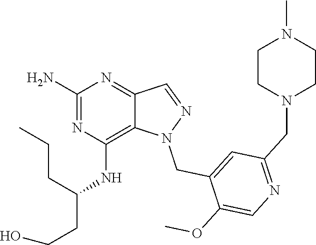 [SMILES: CCC[C@@H](CCO)Nc1nc(N)nc2cnn(Cc3cc(CN4CCN(C)CC4)ncc3OC)c12] (3S)-3-{[5-amino-1-({5-methoxy-2- [(4-methylpiperazin-1-yl)methyl]- pyridin-4-yl}methyl)-1H- pyrazolo[4,3-d]pyrimidin-7- yl]amino}hexan-1-ol | 750.5 | 34.5 | LC/MS [M + H]⁺ = 484.4 LC RT (min/condition) = 0.85/E (¹H NMR 400 MHz, DMSO-d₆) δ = 8.24 (s, 1H), 7.64 (s, 1H), 6.14 (s, 1H), 5.98 (d, J = 9.0 Hz, 1H), 5.85-5.56 (m, 4H), 4.27 (br dd, J = 2.0, 7.1 Hz, 1H), 3.94 (s, 3H), 2.11 (s, 8H), 1.91 (s, 2H), 1.65- 1.49 (m, 2H), 1.42-1.29 (m, 2H), 1.02- 0.85 (m, 2H), 0.76-0.67 (m, 3H) |
| 178 | 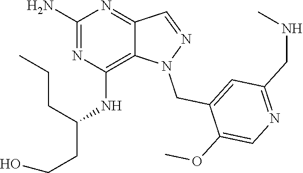 [SMILES: CCC[C@@H](CCO)Nc1nc(N)nc2cnn(Cc3cc(CNC)ncc3OC)c12] (3S)-3-{[5-amino-1-({5-methoxy-2- [(methylamino)methyl]pyridin-4- yl}methyl)-1H-pyrazolo[4,3- d]pyrimidin-7-yl]amino}hexan-1-ol | 616.5 | 33.9 | LC/MS [M + H]⁺ = 415.3 LC RT (min/condition) = 0.93/E (¹H NMR 400 MHz, DMSO-d₆) δ = 8.29 (s, 1H), 7.61 (s, 1H), 6.38 (s, 1H), 5.92 (d, J = 8.3 Hz, 1H), 5.79-5.71 (m, 1H), 5.68-5.60 (m, 3H), 4.30 (br s, 1H), 3.95 (s, 3H), 3.56 (s, 2H), 3.35-3.34 (m, 2H), 3.02 (br d, J = 7.3 Hz, 1H), 2.15 (s, 3H), 1.90 (s, 1H), 1.68 -1.52 (m, 2H), 1.44-1.37 (m, 2H), 1.14- 1.02 (m, 2H), 0.79-0.72 (m, 3H) |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### Industrial Applicability and Uses

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy | Alternative | |
|---|---|---|
| Agent | Name(s) | Target |
| AMG 557 | | B7RP-1 |
| | | (ICOSL) |
| AMP-224 | | PD-1 |
| Atezolizumab | MPDL3280A, | PD-L1 |
| | RO5541267, | |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS |
| | | (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, | CTLA-4 |
| | CP-675, CP- | |
| | 675, 206 | |
| Urelumab | BMS-663513 | CD137 |
| Utomilumab | PF-05082566 | CD137 |
| Varlilumab | CDX 1127 | CD27 |
| Vonlerolizumab | RG7888, | OX40 |
| | MOXR0916, | |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and/or analytical (LC/MS) liquid chromatography methods were used.

LC/MS Condition A: Column: Waters XBridge C18, 2.1 mm×50 mm, 1.7 μm particles; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0% B to 100% B over 3 min, then a 0.50 min hold at 100% B; Flow: 1 mL/min; Detection: MS and UV (220 nm).

LC/MS Condition B: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm).

LC/MS Condition C: Column: Waters XBridge C18, 2.1 mm×50 mm, 1.7 μm particles; Mobile Phase A: acetonitrile with 0.1% TFA; Mobile Phase B: water with 0.1% TFA; Temperature: 37° C.; Gradient: 0% B to 100% B over 3 min, then a 0.50 min hold at 100% B; Flow: 1 mL/min; Detection: MS and UV (240 nm).

LC/MS Condition D: Column: Waters XBridge C18, 2.1 mm×50 mm, 1.7 μm particles; Mobile Phase A: acetonitrile with 0.1% formic acid; Mobile Phase B: water with 0.1% formic acid; Temperature: 37° C.; Gradient: 0% B to 100% B over 2.5 min, then a 0.50 min hold at 100% B; Flow: 1 mL/min; Detection: MS and UV (240 nm).

LC/MS Condition E: Column: Waters X-Bridge BEH C18 XP (50×2.1 mm) 2.5 am; Mobile Phase A: 5:95 acetonitrile: water with 10 mM NH₄OAc; Mobile Phase B:95:5 acetonitrile: water with 10 mM NH₄OAc; Temperature: 50° C.; Gradient: 0-100% B over 3 minutes; Flow: 1.1 ml/min).

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown for convenience, but it is to be understood that they are present in the initial product mixture and separated at a later time, for example by preparative HPLC.

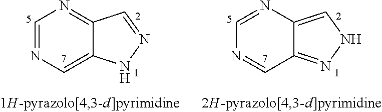 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present disclosure can be prepared by a number of methods well known to one skilled in the art of synthetic organic chemistry. These methods include those described below, or variations thereof. Preferred methods include, but are not limited to, those described below in the Schemes below. The Schemes are intended to be generic, but in some instances specific groups (e.g., methyl ester or methoxy) are depicted for convenience.

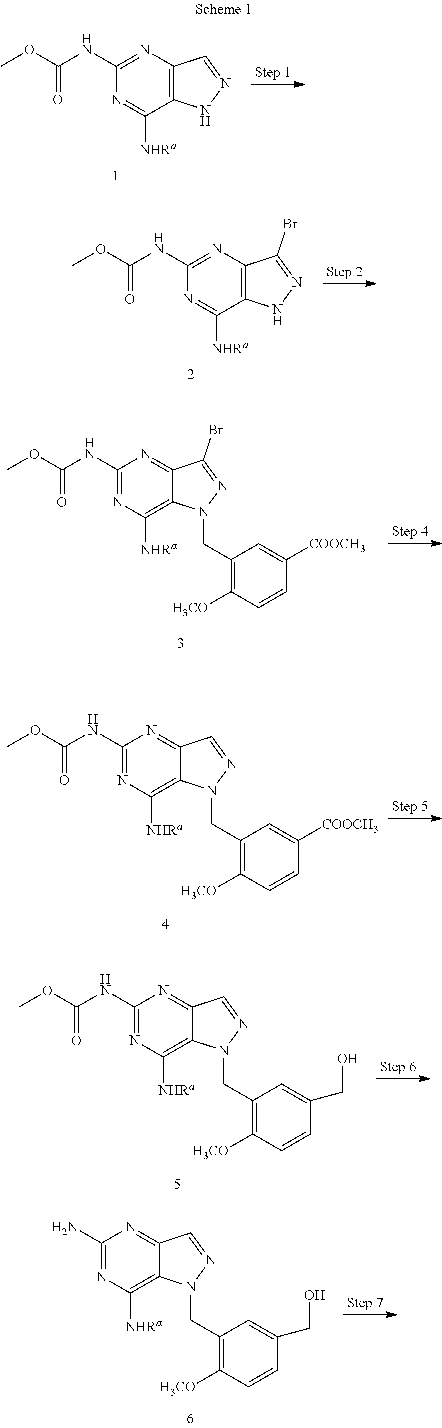

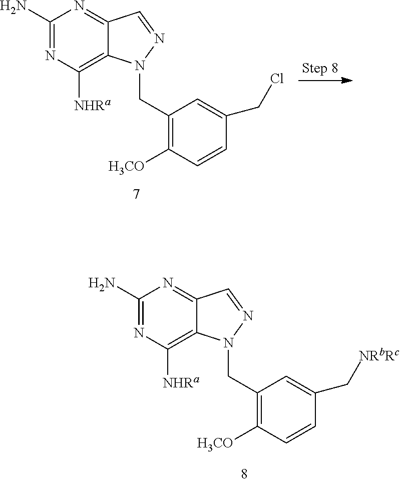

R^{a} can be, in Scheme 1 and other occurrences thereof, for example,

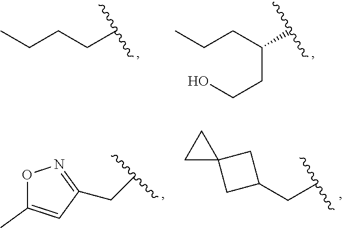

or other suitable moiety.
R^{b}NHR^{c} is, in Scheme 1 and other occurrences thereof, a primary or secondary amine. R^{a}, R^{b}, and/or R^{c} can have functional groups masked by a protecting group that is removed at the appropriate time during the synthetic process.

Compound 8 can be prepared by a synthetic sequence as outlined in Scheme 1 above. Pyrazolopyrimidine 1 is converted to bromide 2 by reaction with NBS. After alkylation with methyl 3-bromomethyl-4-methoxy benzoate, compound 3 is obtained. Compound 3 is hydrogenated under H₂ to give compound 4. Compound 4 is reduced to alcohol 5 with LiAlH₄. Alcohol 5 is treated with NaOH to provide amine 6. Reaction of amine 6 with SOCl₂ gives chloride 7. In the last step of Scheme 1, Compound 8 is prepared by alkylation of chloride 7 with R^{b}NHR^{c}.

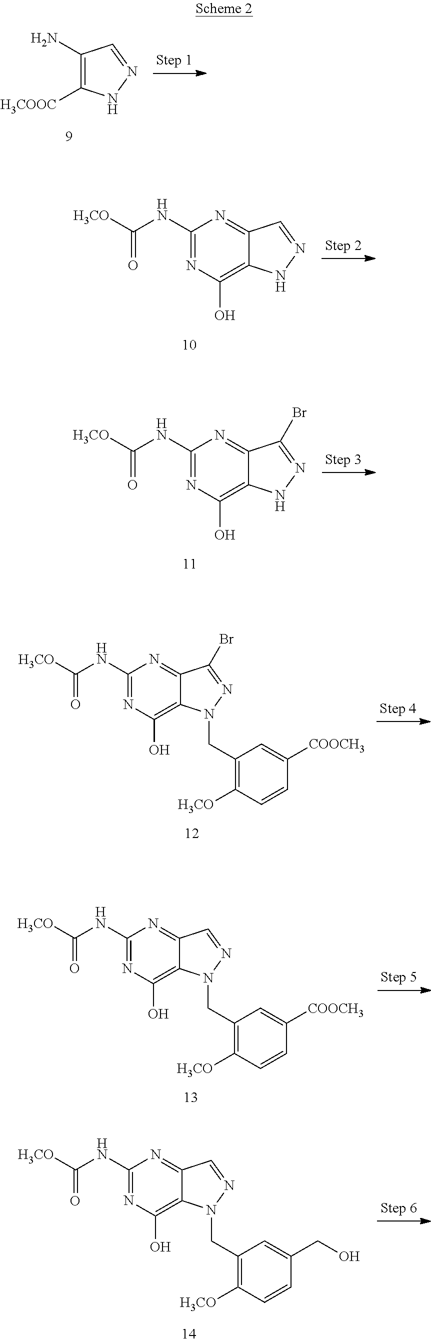

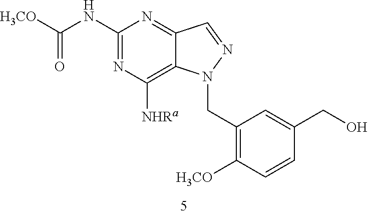

Scheme 2 above shows an alternative method for the preparation of intermediate 5, by coupling methyl 4-amino-1H-pyrazole-5-carboxylate (CAS Reg. No. 923283-54-9) and 1,3-bis(methoxycarbonyl)-2-methyl-2-thiopseudourea (CAS Reg. No. 34840-23-8) to form compound 10. Compound 11 is obtained by bromination of compound 10 with NBS (N-bromosuccinimide). After alkylation with methyl 3-bromomethyl-4-methoxy benzoate, compound 12 is obtained. Compound 12 is hydrogenated under H₂ to give compound 13. Compound 13 is reduced to alcohol 14 by reaction with LiAlH₄. Intermediate 5 is synthesized by reaction of compound 14 with RaNH₂ in the presence of BOP and DBU.

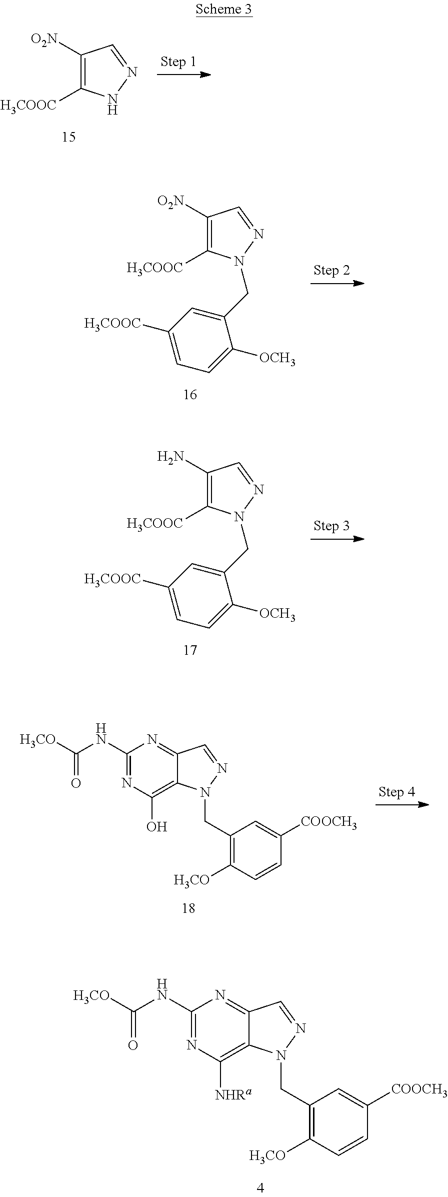

Scheme 3 above shows an alternative method for the preparation of intermediate 4, by alkylation of methyl 4-nitro-1H-pyrazole-5-carboxylate 15 (CAS Reg. No. 1345513-95-2) with methyl 3-bromomethyl-4-methoxy benzoate to form compound 16. Compound 16 is hydrogenated under H₂ to give compound 17. Compound 18 is obtained by reaction of compound 17 with 1,3-bis(methoxycarbonyl)-2-methyl-2-thiopseudourea. Intermediate 4 is synthesized by reaction of compound 18 with RaNH₂ in the presence of BOP and DBU.

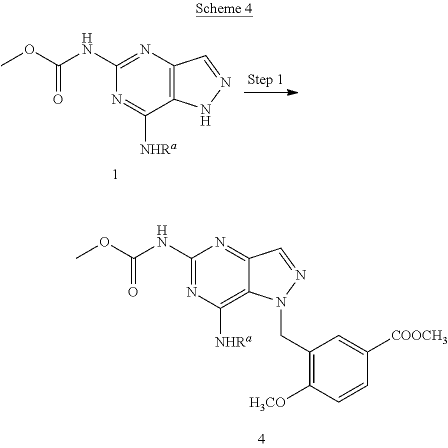

Compound 1 can be alkylated directly with methyl 3-bromomethyl-4-methoxy benzoate to form intermediate 4. However, in this method the ratio of N1 isomer to N2 isomer is generally less favorable.

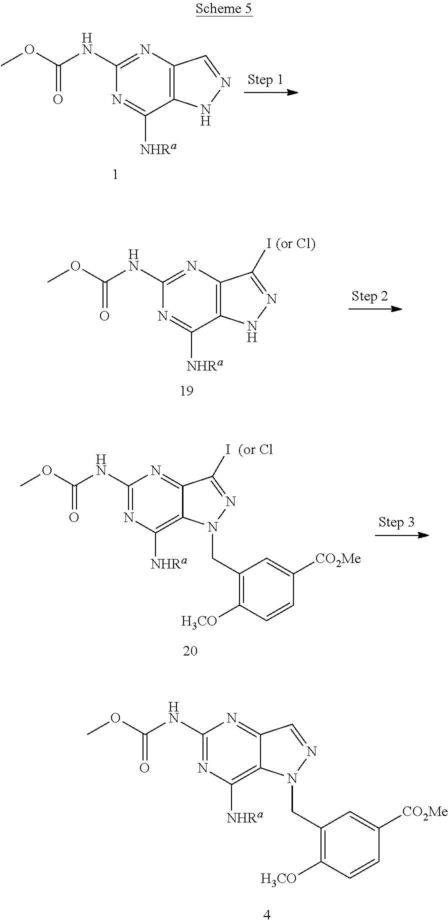

Scheme 5 above shows an alternative method for the preparation of intermediate 4. Pyrazolopyrimidine 1 is converted to iodide or chloride 19 with NIS (N-iodosuccinimide) or NCS (N-chlorosuccinimide). After alkylation with methyl 3-bromomethyl-4-methoxy benzoate, compound 20 is obtained. Compound 20 is hydrogenated under H₂ to give compound 4.

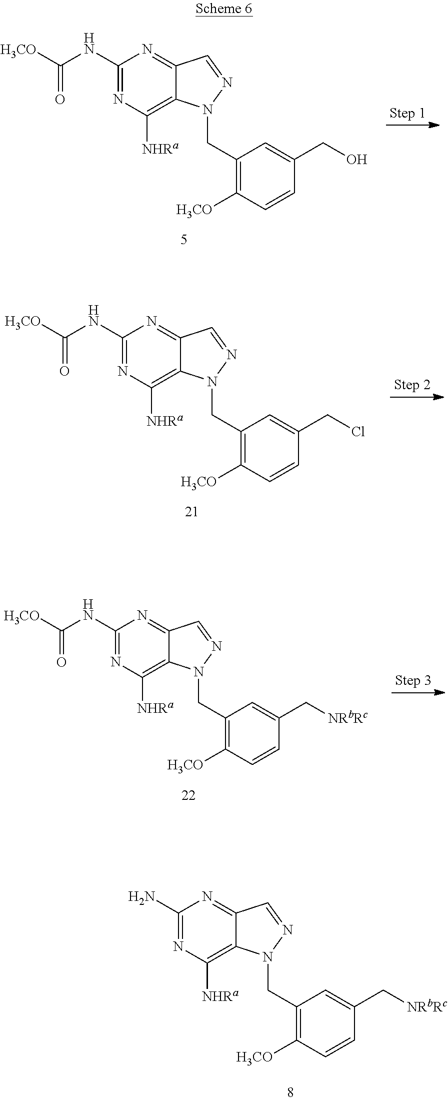

Scheme 6 above shows an alternative method for the preparation of product 8. Reaction of compound 5 with SOCl₂ gives chloride 21. Chloride 7 is treated with R^{b}NHR^{c} to give compound 22. Product 8 is obtained by deprotection of compound 22 with NaOH.

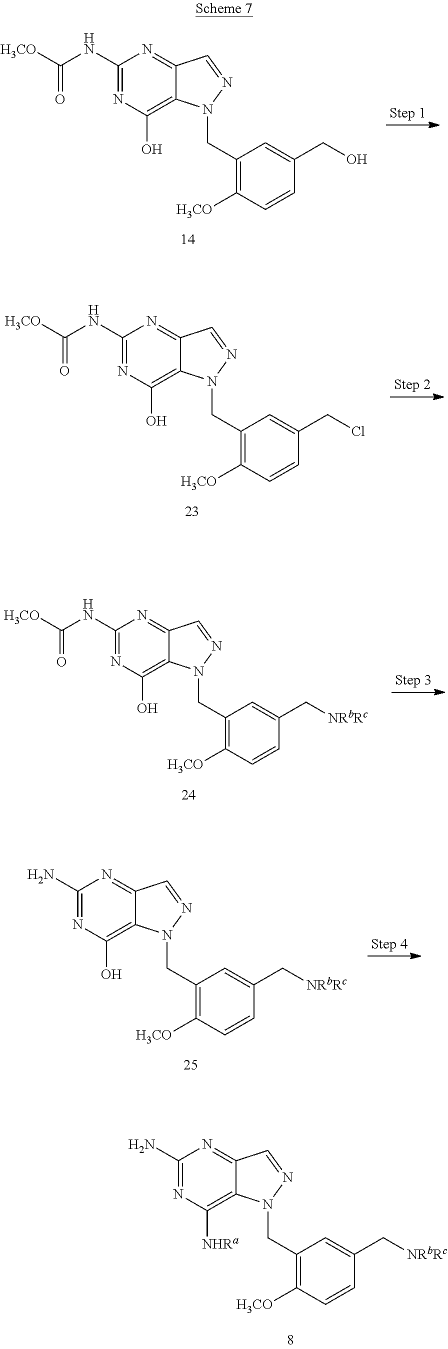

Scheme 7 above shows an alternative method for the preparation of product 8. Reaction of compound 14 with SOCl₂ gives chloride 23. Chloride 7 is treated with R^{b}NHR^{c} to give compound 24. Compound 25 is obtained by deprotection of compound 24 with NaOH. Product 8 is synthesized by reaction of compound 25 with R^{a}NH₂ in the presence of BOP and DBU.

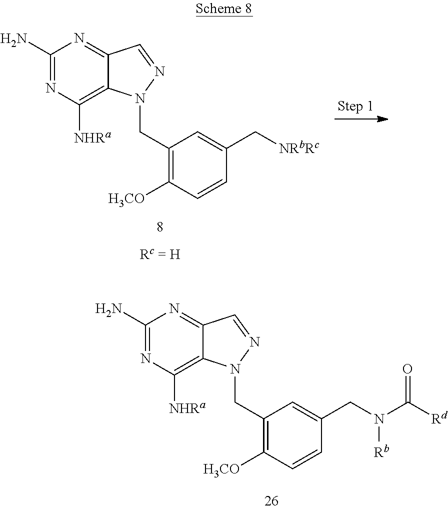

Compound 26 can be prepared by coupling compound 8 (in the instance in which R^{c} is H with acid R^{d}COOH, as outlined in Scheme 8 above.

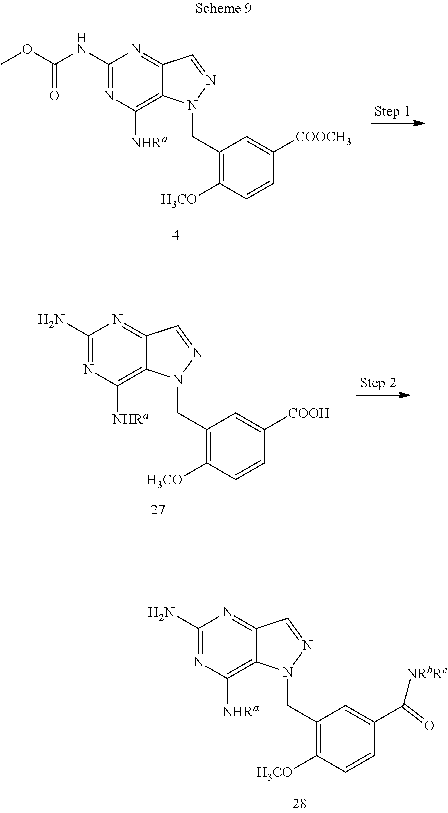

Compound 28 can be prepared by a synthetic sequence outlined in Scheme 9 above. Compound 4 was hydrolyzed using NaOH to form acid 27. Coupling compound 27 with R^{b}NHR^{c} gives product 28.

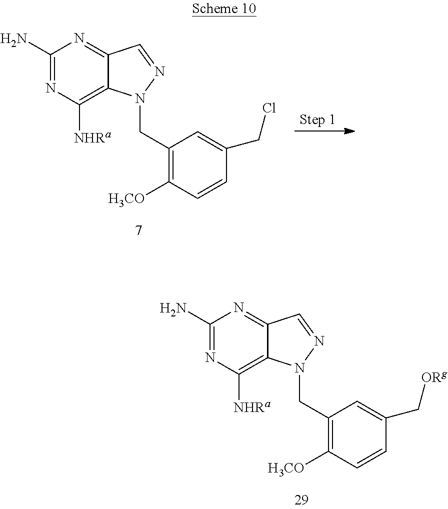

Compound 29 can be obtained by reaction of chloride 7 with an alcohol R^{g}OH, as outlined in Scheme 10 above.

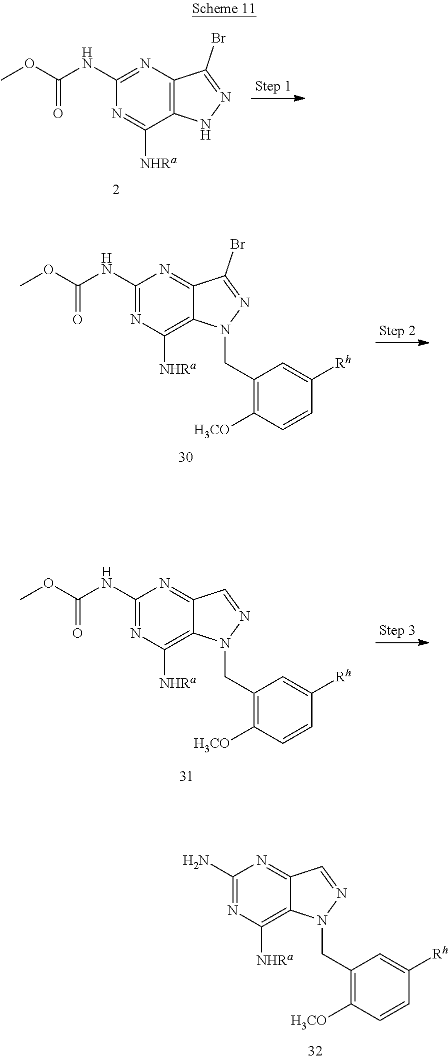

Compound 32 can be prepared by a synthetic sequence outlined in Scheme 11 above. Compound 30 is obtained by alkylation of compound 2. Deprotection of compound 30 gives compound 31. Product 32 is obtained by hydrolysis of compound 31 with NaOH.

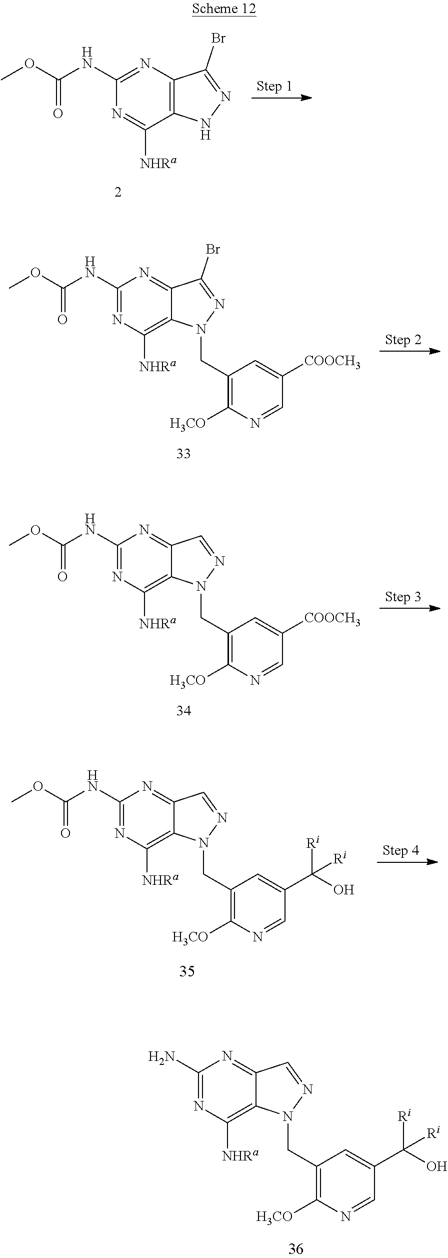

Compound 36 can be prepared by a synthetic sequence outlined in Scheme 12 above. Compound 33 is obtained after alkylation of compound 2. Compound 33 is hydrogenated under H₂ to give compound 34. Compound 34 is converted to compound 35 by reaction with a Grignard reagent RⁱMgBr where Rⁱ is for example lower alkyl. Product 36 is obtained by deprotection of compound 35 using NaOH.

Those skilled in the art will be able to make compounds of this disclosure by referencing the general procedures above, employing reagents, solvents and conditions known in the art, or by modifying the procedures of the specific examples following, mutatis mutandis.

### Synthesis—Specific Examples

To further illustrate the foregoing, the following non-limiting, the following exemplary synthetic schemes are included. Variations of these examples within the scope of the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 101 and higher can be found in Table A1 or Table A2.

### Example A—Compound 105

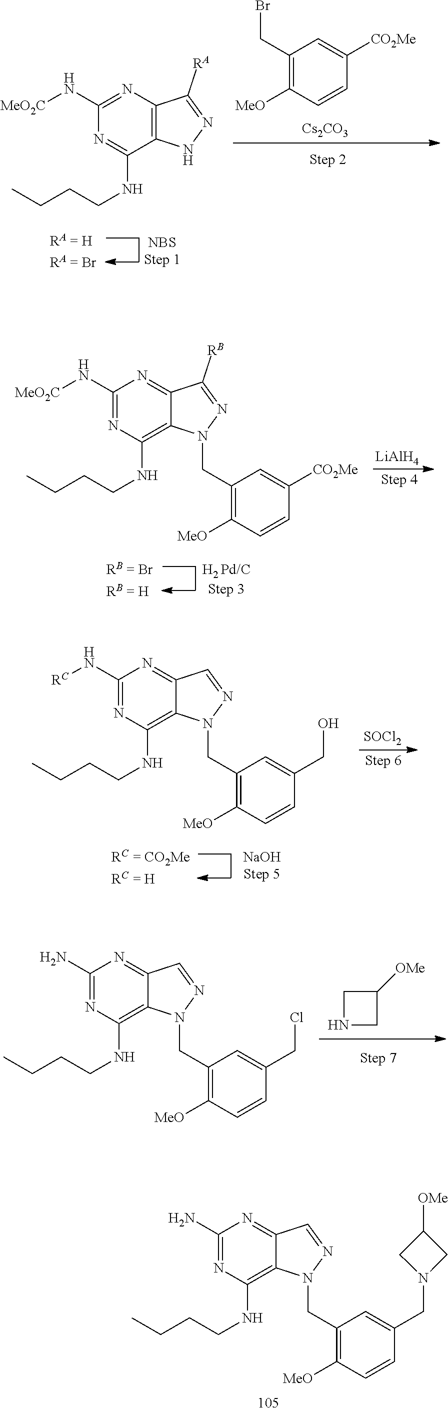

Step 1. To a suspension of methyl (7-(butylamino)-1H-pyrazolo[4,3-d]pyrimidin-5-yl)carbamate (4 g, 15.13 mmol) in DMF (7 mL) was added a solution of NBS (2.96 g, 16.65 mmol) in acetonitrile (14 mL). The reaction mixture was stirred at RT for 1 hour. Water (33 mL) was added. The precipitate was collected by filtration. The solid was washed with water (3×20 mL), and air dried overnight, giving methyl (3-bromo-7-(butylamino)-1H-pyrazolo[4,3-d]pyrimidin-5-yl)carbamate.

LC-MS m/z 343.1 [M+H]⁺.

¹H NMR (400 MHz, DMSO-d₆) δ 12.89 (s, 1H), 9.79 (s, 1H), 7.58 (s, 1H), 3.62 (s, 3H), 3.54 (q, J=6.8 Hz, 2H), 1.68-1.56 (m, 2H), 1.47-1.33 (m, 2H), 0.94 (t, J=7.4 Hz, 3H).

Step 2. Cs₂CO₃ (5.73 g, 17.59 mmol) was added to a mixture of methyl (3-bromo-7-(butylamino)-1H-pyrazolo[4,3-d]pyrimidin-5-yl)carbamate (3.32 g, 9.67 mmol) and methyl 3-(bromomethyl)-4-methoxybenzoate (2.279 g, 8.79 mmol) in DMF (21.72 ml) at RT. The reaction mixture was stirred at RT for 2 h, diluted with EtOAc, washed with water, dried, filtered, and concentrated. The crude product was purified on a silica gel column with 0-20% EtOAc in hexanes to provide methyl 3-((3-bromo-7-(butylamino)-5-((methoxycarbonyl)amino)-1H-pyrazolo[4,3-d]pyrimidin-1-yl)methyl)-4-methoxybenzoate as a white solid.

LC-MS m/z 521.1 [M+H]⁺.

¹H NMR (400 MHz, DMSO-d6) δ 9.87 (s, 1H), 7.98-7.90 (m, 1H), 7.50 (d, J=2.2 Hz, 1H), 7.40 (t, J=5.6 Hz, 1H), 7.16 (d, J=8.7 Hz, 1H), 5.75 (s, 2H), 3.83 (s, 3H), 3.78 (s, 3H), 3.63 (s, 3H), 3.55 (q, J=6.6 Hz, 2H), 1.65-1.53 (m, 2H), 1.31-1.23 (m, 2H), 0.87 (t, J=7.4 Hz, 3H).

Step 3. Pd/C (10 wt %, 30 mg, 0.403 mmol) was added to a solution of methyl 3-((3-bromo-7-(butylamino)-5-((methoxycarbonyl)amino)-1H-pyrazolo[4,3-d]pyrimidin-1-yl)methyl)-4-methoxybenzoate (0.21 g, 0.403 mmol) in MeOH (5 mL) at RT. The reaction mixture was stirred under H₂ overnight. The catalyst was filtered off, and the filtrate was concentrated to afford methyl 3-((7-(butylamino)-5-((methoxycarbonyl)amino)-1H-pyrazolo[4,3-d]pyrimidin-1-yl)methyl)-4-methoxybenzoate as a white solid.

LC-MS m/z 443.2 [M+H]⁺.

¹H NMR (400 MHz, Chloroform-d) δ 8.77 (t, J=5.8 Hz, 1H), 8.09 (s, 1H), 7.96 (dd, J=8.8, 2.2 Hz, 1H), 7.77 (d, J=2.1 Hz, 1H), 6.90 (d, J=8.7 Hz, 1H), 6.03 (s, 2H), 3.93-3.75 (m, 11H), 1.73-1.63 (m, 2H), 1.31 (h, J=7.6 Hz, 2H), 0.89 (t, J=7.4 Hz, 3H).

Step 4. LiAlH₄ in THF (1M) (1.549 mL, 1.549 mmol) was added to a mixture of methyl 3-((7-hydroxy-5-((methoxycarbonyl)amino)-1H-pyrazolo[4,3-d]pyrimidin-1-yl)methyl)-4-methoxybenzoate (60 mg, 0.155 mmol) in THF (8 mL) at 0° C. The reaction mixture was stirred at RT for 3 h, quenched by the slow addition of methanol and stirred with Rochelle salt (1M, 3 mL) for 1 h. The aqueous solution was extracted with EtOAC. The combined organic layers were dried, filtered, and concentrated. The crude product was purified on a silica gel column with 0-20% MeOH in DCM to provide methyl (7-(butylamino)-1-(5-(hydroxymethyl)-2-methoxybenzyl)-1H-pyrazolo[4,3-d]pyrimidin-5-yl)carbamate as a white solid.

LC-MS m/z 443.2 [M+H]⁺.

¹H NMR (400 MHz, Chloroform-d) δ 8.08 (s, 1H), 7.78 (s, 1H), 7.33-7.26 (m, 1H), 6.96 (d, J=2.1 Hz, 1H), 6.89 (d, J=8.5 Hz, 1H), 5.70 (t, J=5.4 Hz, 1H), 5.57 (s, 2H), 5.29 (s, 2H), 4.47 (s, 2H), 3.90 (s, 3H), 3.73 (s, 3H), 3.44 (td, J=7.0, 5.3 Hz, 3H), 1.52-1.39 (m, 2H), 1.29-1.15 (m, 2H), 0.87 (t, J=7.4 Hz, 3H).

Step 5. NaOH (10M, 5.02 mL, 50.2 mmol) was added to a mixture of methyl (7-(butylamino)-1-(5-(hydroxymethyl)-2-methoxybenzyl)-1H-pyrazolo[4,3-d]pyrimidin-5-yl)carbamate (1.04 g, 2.509 mmol) in dioxane (25 mL) at RT. The reaction mixture was heated at 54° C. overnight, diluted with water, and extracted with EtOAc. The combined organic layers were dried, filtered, and concentrated. The crude product was purified on a silica gel column with 0-30% MeOH in DCM to provide Compound 140 as a white solid.

LC-MS m/z 357.2 [M+H]⁺.

Step 6. SOCl₂ (0.410 ml, 5.61 mmol) was added to a solution of (3-((5-amino-7-(butylamino)-1H-pyrazolo[4,3-d]pyrimidin-1-yl)methyl)-4-methoxyphenyl)methanol (0.1 g, 0.281 mmol) in THF (4.60 ml) at RT. The reaction mixture was stirred at RT for 2 h. The solvent was evaporated to afford N⁷-butyl-1-(5-(chloromethyl)-2-methoxybenzyl)-1H-pyrazolo[4,3-d]pyrimidine-5,7-diamine as a white solid.

LC-MS m/z 375.2 [M+H]⁺.

Step 7. A mixture of N⁷-butyl-1-(5-(chloromethyl)-2-methoxybenzyl)-1H-pyrazolo[4,3-d]pyrimidine-5,7-diamine (10 mg, 0.027 mmol) and 3-methoxyazetidine (13.94 mg, 0.160 mmol) in DMF (0.5 mL) was stirred at RT overnight. The crude product was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing Compound 105 were combined and dried via centrifugal evaporation.

The following compounds were analogously prepared: Compound 101, Compound 102, Compound 103, Compound 104, Compound 106, Compound 107, Compound 108, Compound 109, Compound 110, Compound 111, Compound 112, Compound 113, Compound 114, Compound 115, Compound 116, Compound 117, Compound 118, Compound 119, Compound 120, Compound 121, Compound 122, Compound 123, Compound 124, Compound 125, Compound 126, Compound 127, Compound 129, Compound 130, Compound 131, Compound 132, Compound 133, Compound 134, Compound 135, Compound 137, Compound 138, Compound 142, and Compound 151.

### Example B—Compound 128

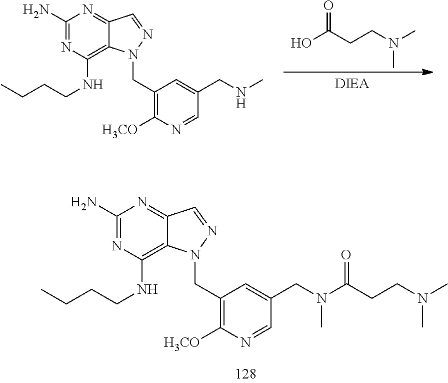

DIEA (6.07 μl, 0.035 mmol) was added to a mixture of N7-butyl-1-(2-methoxy-5-((methylamino)methyl)benzyl)-1H-pyrazolo[4,3-d]pyrimidine-5,7-diamine (Compound 132, 9.9 mg, 0.027 mmol), 3-(dimethylamino)propanoic acid (3.45 mg, 0.029 mmol) and HATU (12.22 mg, 0.032 mmol) in DMF (1 mL) at RT. The reaction mixture was stirred at RT overnight. The crude material was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing Compound 128 were combined and dried via centrifugal evaporation.

The following compounds were analogously prepared: Compound 136, Compound 146, Compound 147, and Compound 148.

### Example C—Compound 139

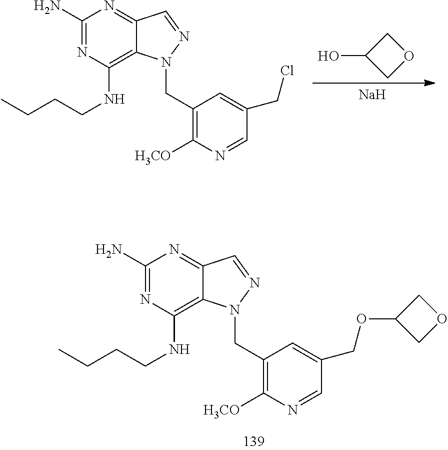

NaH (60%) (6.40 mg, 0.160 mmol) was added to a solution of oxetan-3-ol (11.86 mg, 0.160 mmol) in DMF (0.5 mL) at RT. The mixture was stirred at RT for 10 min and added to a solution of N⁷-butyl-1-(5-(chloromethyl)-2-methoxybenzyl)-1H-pyrazolo[4,3-d]pyrimidine-5,7-diamine (10 mg, 0.027 mmol) in DMF (0.5 mL) at RT. The reaction mixture was stirred at RT for 2 h. The crude product was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 100% B; Flow Rate: 20 mL/min; Column Temperature: 25° C. Fractions containing Compound 139 (collection triggered by MS and UV signals) were combined and dried via centrifugal evaporation.

The following compounds were analogously prepared: Compound 141, Compound 143, and Compound 144.

### Example D—Compound 145

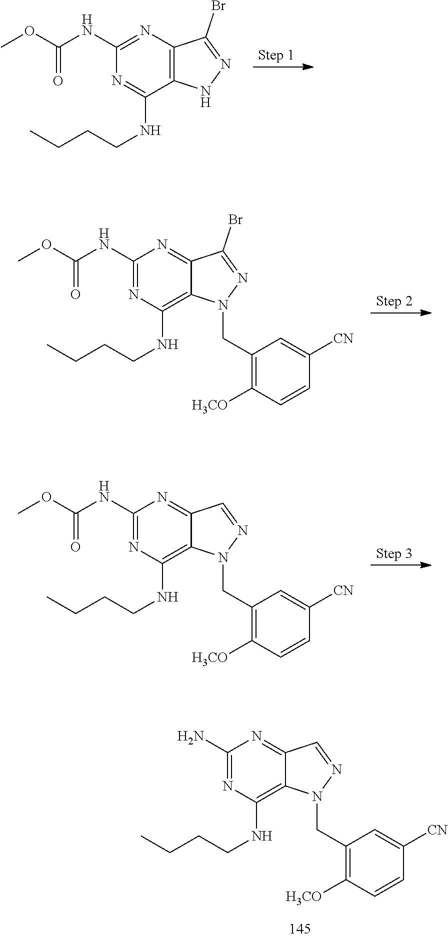 [SMILES: CCCCNc1nc(NC(=O)OC)nc2c(Br)n[nH]c12 ; CCCCNc1nc(NC(=O)OC)nc2c(Br)nn(Cc3cc(C#N)ccc3OC)c12 ; CCCCNc1nc(NC(=O)OC)nc2cnn(Cc3cc(C#N)ccc3OC)c12 ; CCCCNc1nc(N)nc2cnn(Cc3cc(C#N)ccc3OC)c12]

Step 1. Cs₂CO₃ (0.380 g, 1.166 mmol) was added to a mixture of methyl (3-bromo-7-(butylamino)-1H-pyrazolo[4,3-d]pyrimidin-5-yl)carbamate (0.2 g, 0.583 mmol) and 3-(bromomethyl)-4-methoxybenzonitrile (0.132 g, 0.583 mmol) in DMF (2 mL) at RT. The reaction mixture was stirred at RT over a weekend. The reaction mixture was diluted with EtOAc, washed with water, dried, filtered, and concentrated. The crude product was purified on a silica gel column with 0-70% EtOAc in hexanes to give methyl (3-bromo-7-(butylamino)-1-(4-cyano-2-methoxybenzyl)-1H-pyrazolo[4,3-d]pyrimidin-5-yl)carbamate as a white solid.

LC-MS m/z 488.1 [M+H]⁺.

Step 2. A mixture of methyl (3-bromo-7-(butylamino)-1-(5-cyano-2-methoxybenzyl)-1H-pyrazolo[4,3-d]pyrimidin-5-yl)carbamate (81 mg, 0.166 mmol) and Pd/C 10 wt % (20 mg, 0.166 mmol) in methanol (2 mL) was stirred under H₂ overnight. After the catalyst was filtered off, the filtrate was concentrated to afford methyl (7-(butylamino)-1-(4-cyano-2-methoxy-benzyl)-1H-pyrazolo[4,3-d]pyrimidin-5-yl)carbamate as a white solid.

LC-MS m/z 410.2 [M+H]⁺.

Step 3. A mixture of methyl (7-(butylamino)-1-(5-cyano-2-methoxybenzyl)-1H-pyrazolo[4,3-d]pyrimidin-5-yl)carbamate (42.6 mg, 0.104 mmol) and 10N NaOH (0.208 mL, 2.081 mmol) in dioxane (1.5 mL) was stirred at 54° C. overnight. The crude product was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing Compound 145 were combined and dried via centrifugal evaporation.

### Example E—Compound 149

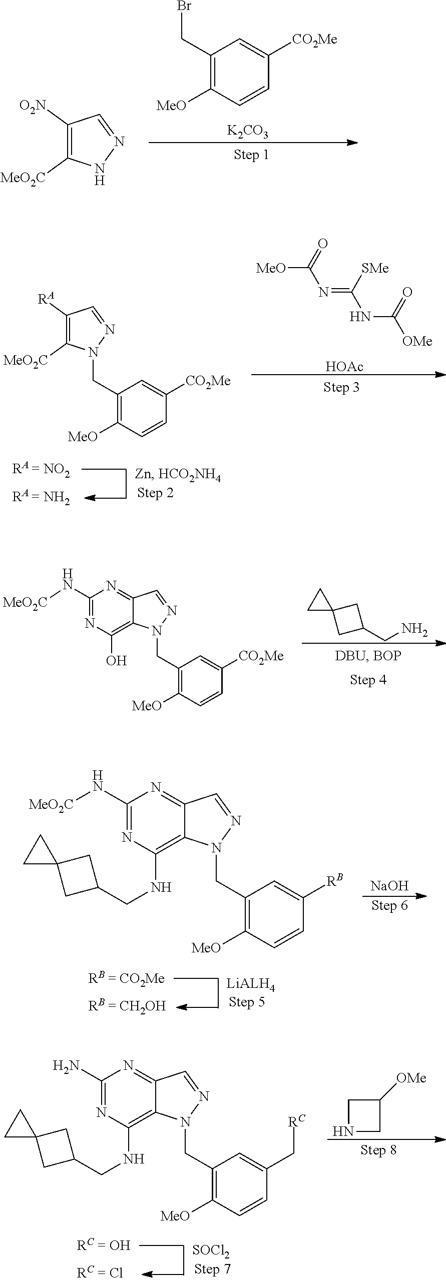

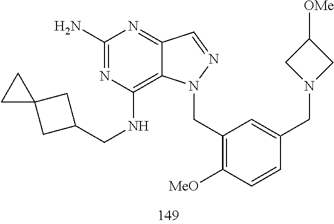 [SMILES: COc1ccc(CN2CC(OC)C2)cc1Cn1ncc2nc(N)nc(NCC3CC4(CC4)C3)c21]

Step 1. A mixture of methyl 3-(bromomethyl)-4-methoxybenzoate (3.6 g, 13.89 mmol), methyl 4-nitro-1H-pyrazole-5-carboxylate (2.377 g, 13.89 mmol) and K₂CO₃ (2.496 g, 18.06 mmol) in DMF (30 mL) was stirred at RT for 3 h. The reaction mixture was diluted with water and extracted with EtOAc. The combined organic layers were dried, filtered, and concentrated. The crude product was purified on a silica gel column with 0-50% EtOAc in hexanes to give methyl 1-(2-methoxy-5-(methoxycarbonyl)benzyl)-4-nitro-1H-pyrazole-5-carboxylate as a white solid.

LC-MS m/z 350.1 [M+H]⁺.

¹H NMR (400 MHz, DMSO-d6) δ 8.38 (s, 1H), 7.98 (dd, J=8.6, 2.2 Hz, 1H), 7.89 (d, J=2.2 Hz, 1H), 7.15 (d, J=8.7 Hz, 1H), 5.52 (s, 2H), 3.98 (s, 3H), 3.82 (d, J=5.1 Hz, 6H).

Step 2. To a mixture of methyl 1-(2-methoxy-5-(methoxycarbonyl)benzyl)-4-nitro-1H-pyrazole-5-carboxylate (1 g, 2.86 mmol) and ammonium formate (0.903 g, 14.31 mmol) in THF (9 mL) and MeOH (9 mL) was added Zn (0.599 g, 9.16 mmol) at RT. The reaction mixture was stirred at RT for 1 h. The solid was filtered off. The filtrate was concentrated to yield methyl 4-amino-1-(2-methoxy-5-(methoxycarbonyl)benzyl)-1H-pyrazole-5-carboxylate as a white solid. LC-MS m/z 320.1 [M+H]⁺.

Step 3. A mixture of 1,3-bis(methoxycarbonyl)-2-Methyl-2-thiopseudourea (0.452 g, 2.192 mmol) and methyl 4-amino-1-(2-methoxy-5-(methoxycarbonyl)benzyl)-1H-pyrazole-5-carboxylate (0.7 g, 2.192 mmol) was taken up in MeOH (18 mL) and treated with acetic acid (0.627 mL, 10.96 mmol) at RT. The reaction mixture was stirred overnight. Sodium methoxide in methanol (4.37M) (5.02 mL, 21.92 mmol) was then added to the reaction mixture, which was then stirred at RT overnight. The pH was adjusted to 5 by the slow addition of acetic acid. The precipitate was collected by filtration, washed with water and acetonitrile, and dried to provide methyl 3-((7-hydroxy-5-((methoxycarbonyl)amino)-1H-pyrazolo[4,3-d]pyrimidin-1-yl)methyl)-4-methoxybenzoate as a white solid.

LC-MS m/z 388.1 [M+H]⁺.

Step 4. A solution of spiro[2.3]hexan-5-ylmethanamine (0.201 g, 1.808 mmol), methyl 3-((7-hydroxy-5-((methoxycarbonyl)amino)-1H-pyrazolo[4,3-d]pyrimidin-1-yl)methyl)-4-methoxybenzoate (0.35 g, 0.904 mmol) in DMSO (5 mL) was treated with DBU (0.545 mL, 3.61 mmol) and BOP (0.799 g, 1.807 mmol). The reaction mixture was heated at 40° C. for 1 h. Water was added to quench the reaction. The aqueous solution was extracted with EtOAc. The combined organic layers were dried, filter, and concentrated. The crude product was purified on a silica gel column with 0-20% MeOH in DCM to afford methyl 4-methoxy-3-((5-((methoxycarbonyl)amino)-7-((spiro[2.3]hexan-5-ylmethyl)amino)-1H-pyrazolo[4,3-d]pyrimidin-1-yl)methyl)benzoate a white solid.

LC-MS m/z 481.2 [M+H]⁺.

¹H NMR (400 MHz, DMSO-d6) δ 9.65 (s, 1H), 7.96-7.87 (m, 2H), 7.25 (d, J=2.2 Hz, 1H), 7.23-7.03 (m, 2H), 5.76 (s, 2H), 3.88 (d, J=6.6 Hz, 3H), 3.74 (s, 3H), 3.69-3.52 (m, 5H), 2.84-2.68 (m, 1H), 2.08-1.90 (m, 2H), 1.86-1.77 (m, 2H), 0.34 (s, 4H).

Step 5. A solution of methyl 4-methoxy-3-((5-((methoxycarbonyl)amino)-7-((spiro[2.3]hexan-5-ylmethyl)amino)-1H-pyrazolo[4,3-d]pyrimidin-1-yl)methyl)benzoate (0.122 g, 0.254 mmol) in THF (3 mL) was cooled to 0° C. and was treated with LiAlH₄ (0.127 mL, 0.254 mmol) dropwise. After 20 min, the reaction was quenched by slow addition of methanol and was stirred with Rochelle salt (1M, 3 mL) for 1 h. The aqueous solution was extracted with EtOAC. The combined organic layers were dried, filtered, and concentrated. The crude product was purified on a silica gel column with 0-30% MeOH in DCM to afford methyl (1-(5-(hydroxymethyl)-2-methoxybenzyl)-7-((spiro[2.3]hexan-5-ylmethyl)amino)-1H-pyrazolo[4,3-d]pyrimidin-5-yl)carbamate as a white solid.

LC-MS m/z 453.2 [M+H]⁺.

Step 6. NaOH (10N, 0.350 mL, 3.50 mmol) was added to a mixture of methyl (1-(5-(hydroxymethyl)-2-methoxybenzyl)-7-((spiro[2.3]hexan-5-ylmethyl)amino)-1H-pyrazolo[4,3-d]pyrimidin-5-yl)carbamate (79.1 mg, 0.175 mmol) in dioxane (2 mL) and DMSO (1 mL) at RT. The reaction mixture was heated at 54° C. overnight. The reaction mixture was diluted with water and extracted with EtOAc. The combined organic layers were dried, filtered, and concentrated. The crude product was purified on a silica gel column with 0-30% MeOH in DCM to afford (3-((5-amino-7-((spiro[2.3]hexan-5-ylmethyl)amino)-1H-pyrazolo[4,3-d]pyrimidin-1-yl)methyl)-4-methoxyphenyl)methanol as a white solid.

LC-MS m/z 395.2 [M+H]⁺.

Step 7. SOCl₂ (0.221 mL, 3.04 mmol) was added to a solution of (3-((5-amino-7-((spiro[2.3]hexan-5-ylmethyl)amino)-1H-pyrazolo[4,3-d]pyrimidin-1-yl)methyl)-4-methoxy-phenyl)methanol (60 mg, 0.152 mmol) in THF (1.5 mL) at RT. The reaction mixture was stirred at RT for 2 h. The solvent was evaporated off to afford 1-(5-(chloromethyl)-2-methoxybenzyl)-N7-(spiro[2.3]hexan-5-ylmethyl)-1H-pyrazolo[4,3-d]pyrimidine-5,7-diamine as a white solid. LC-MS m/z 413.2 [M+H]⁺.

Step 8. A mixture of 1-(5-(chloromethyl)-2-methoxybenzyl)-N7-(spiro[2.3]hexan-5-ylmethyl)-1H-pyrazolo[4,3-d]pyrimidine-5,7-diamine (10 mg, 0.024 mmol) and 3-methoxyazetidine (2.110 mg, 0.024 mmol) in DMF (0.5 mL) was stirred at RT for 2 h. The crude material was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing Compound 149 were combined and dried via centrifugal evaporation.

Compound 150 was prepared analogously per this Example.

### Example F—Compound 152

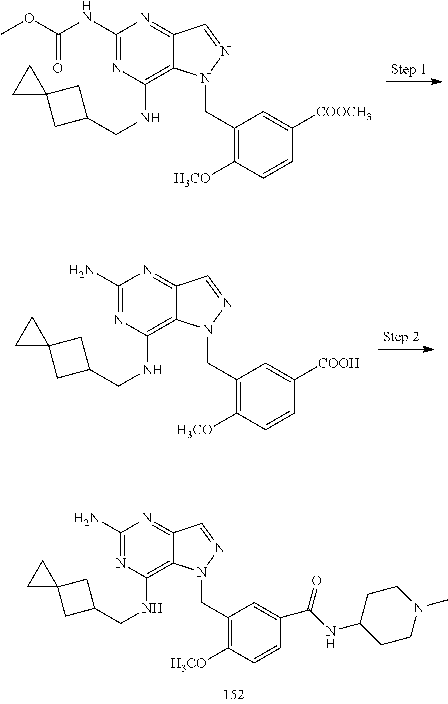 [SMILES: COC(=O)Nc1nc(NCC2CC3(CC3)C2)c2c(cnn2Cc2cc(C(=O)OC)ccc2OC)n1 ; COc1ccc(C(=O)O)cc1Cn1ncc2nc(N)nc(NCC3CC4(CC4)C3)c21 ; COc1ccc(C(=O)NC2CCN(C)CC2)cc1Cn1ncc2nc(N)nc(NCC3CC4(CC4)C3)c21]

Step 1. NaOH (10N, 0.237 mL, 2.372 mmol) was added to methyl 4-methoxy-3-((5-((methoxycarbonyl)amino)-7-((spiro[2.3]hexan-5-ylmethyl)amino)-1H-pyrazolo[4,3-d]pyrimidin-1-yl)methyl)benzoate (57 mg, 0.119 mmol) in DMSO (1 mL) at RT. The reaction mixture was heated at 54° C. overnight and neutralized by addition of 6 N HCl. The solvent was evaporated off to afford 3-((5-amino-7-((spiro[2.3]hexan-5-ylmethyl)amino)-1H-pyrazolo[4,3-d]pyrimidin-1-yl)methyl)-4-methoxybenzoic acid as a white solid.

LC-MS m/z 409.2 [M+H]⁺.

Step 2. DIEA (8.53 μl, 0.049 mmol) was added to a mixture of 1-methylpiperidin-4-amine (16.77 mg, 0.147 mmol), 3-((5-amino-7-((spiro[2.3]hexan-5-ylmethyl)amino)-1H-pyrazolo[4,3-d]pyrimidin-1-yl)methyl)-4-methoxybenzoic acid (10 mg, 0.024 mmol) and HATU (12.10 mg, 0.032 mmol) in DMF (0.5 mL) at RT. The reaction mixture was stirred at RT for 2 h. The crude material was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing Compound 152 were combined and dried via centrifugal evaporation.

Compound 153 was analogously prepared per this Example.

### Example G—Compound 154

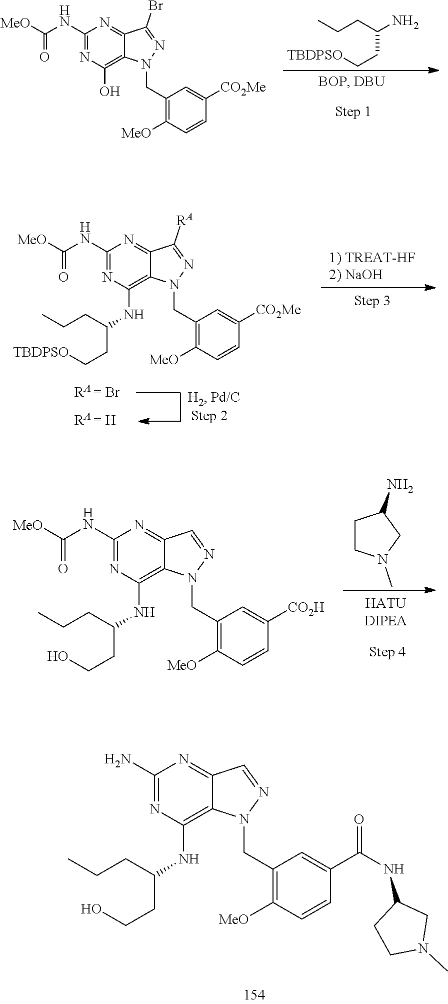

Step 1. A mixture of methyl 3-((3-bromo-7-hydroxy-5-((methoxycarbonyl)amino)-1H-pyrazolo[4,3-d]pyrimidin-1-yl)methyl)-4-methoxybenzoate (0.5 g, 1.072 mmol) in DMSO (5 mL) was treated with (S)-1-((tert-butyldiphenylsilyl)oxy)hexan-3-amine (0.763 g, 2.145 mmol), 2,3,4,6,7,8,9,10-octahydropyrimido[1,2-a]azepine (0.5 mL, 3.32 mmol) followed by ((1H-benzo[d][1,2,3]triazol-1-yl)oxy)tris(dimethylamino)phosphonium hexafluorophosphate(V) (0.949 g, 2.145 mmol). The reaction mixture was heated at 70° C. for 2 h, diluted with EtOAc, and washed with water. The solvent mixture was dried over Na₂SO₄ and the solvent was removed. The residue was diluted with MeOH and filtered to remove starting material. The solvent was removed and the material was purified on silica gel (dry load) Hexane-EtOAc 0-100% to afford methyl (S)-3-((3-bromo-7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxybenzoate (0.59 g, 0.734 mmol, 68.4% yield).

LC-MS m/z 803.3 [M+H]⁺.

¹H NMR (400 MHz, DMSO-d₆) δ 9.84 (s, 1H), 7.98-7.81 (m, 1H), 7.66-7.31 (m, 10H), 7.30-7.19 (m, 2H), 7.14-7.06 (m, 1H), 6.71-6.58 (m, 1H), 5.87-5.59 (m, 2H), 4.77-4.53 (m, 1H), 3.80-3.75 (m, 3H), 3.75-3.71 (m, 3H), 3.67-3.61 (m, 2H), 3.60-3.56 (m, 3H), 1.95-1.77 (m, 2H), 1.64-1.42 (m, 2H), 1.29-1.10 (m, 2H), 0.92 (s, 9H), 0.78 (br t, J=7.3 Hz, 3H)

Step 2. To a Parr bottle was added methyl (S)-3-((3-bromo-7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxybenzoate (0.59 g, 0.734 mmol), methanol (10 mL), and Pd/C (20 mg, 0.188 mmol). The hydrogenation reaction was allowed to proceed for 2 h at 25° C. under 50 psi. The material was filtered and solvent removed to afford methyl (S)-3-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxybenzoate (0.466 g, 0.624 mmol, 85% yield). LC-MS m/z 725.4 [M+H]⁺.

¹H NMR (400 MHz, DMSO-d6) δ 7.76-7.66 (m, 3H), 7.62 (s, 5H), 7.55-7.42 (m, 8H), 7.42-7.33 (m, 2H), 7.31-7.06 (m, 1H), 5.93-5.68 (m, 1H), 3.85-3.68 (m, 5H), 1.98-1.80 (m, 2H), 1.79-1.68 (m, 1H), 1.62-1.39 (m, 3H), 1.38-1.23 (m, 2H), 1.01 (s, 9H), 0.92 (s, 3H), 0.87 (t, J=7.3 Hz, 3H), 0.83-0.77 (m, 1H)

Step 3. A 20 mL scintillation vial was charged with methyl (S)-3-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxybenzoate (460 mg, 0.635 mmol), dioxane (4 mL) and triethylamine trihydrofluoride (TREAT-HF™, 1.3 mL, 7.98 mmol). The reaction mixture was stirred at 50° C. for 2 hours. NaOH (6 mL, 30.0 mmol) was added, followed by stirring at 80° C. for 1 h. After cooling, the reaction mixture was neutralized with 5N HCl, and evaporated to dryness on a V10 evaporator. Flash chromatography (EZ Prep., 50 g column, loaded in DMSO/water, 0 to 60% MeCN in water containing 0.05% TFA over 14 minutes) gave (S)-3-((5-amino-7-((1-hydroxyhexan-3-yl)amino)-1H-pyrazolo[4,3-d]pyrimidin-1-yl)methyl)-4-methoxybenzoic acid (100 mg, 0.241 mmol, 38.0% yield) as a white lyophilized solid.

LC-MS m/z 451.2 [M+H]⁺.

¹H NMR (400 MHz, DMSO-d6) δ 12.74-12.22 (m, 2H), 7.81-7.62 (m, 3H), 7.57 (s, 1H), 7.47-7.35 (m, 1H), 7.16-7.06 (m, 1H), 6.96-6.84 (m, 1H), 5.56 (br d, J=14.7 Hz, 2H), 4.53-4.17 (m, 2H), 3.61 (s, 3H), 1.51 (br d, J=6.4 Hz, 2H), 1.36-1.17 (m, 2H), 1.01-0.80 (m, 2H), 0.56 (t, J=7.4 Hz, 3H).

Step 4. A 20 mL scintillation vial was charged with (S)-3-((5-amino-7-((1-hydroxy-hexan-3-yl)amino)-1H-pyrazolo[4,3-d]pyrimidin-1-yl)methyl)-4-methoxybenzoic acid (30 mg, 0.072 mmol), HATU (33.0 mg, 0.087 mmol), (R)-1-methylpyrrolidin-3-amine (14.50 mg, 0.145 mmol) and DMF (1.5 mL). DIPEA (0.038 mL, 0.217 mmol) was added, and the reaction stirred at RT for 1 hour. The crude product was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 30 100% B; Flow Rate: 20 mL/min; Column Temperature: 25° C. Fraction collection was triggered by MS and UV signals. Fractions containing Compound 154 were combined and dried via centrifugal evaporation.

### Example H—Compound 157

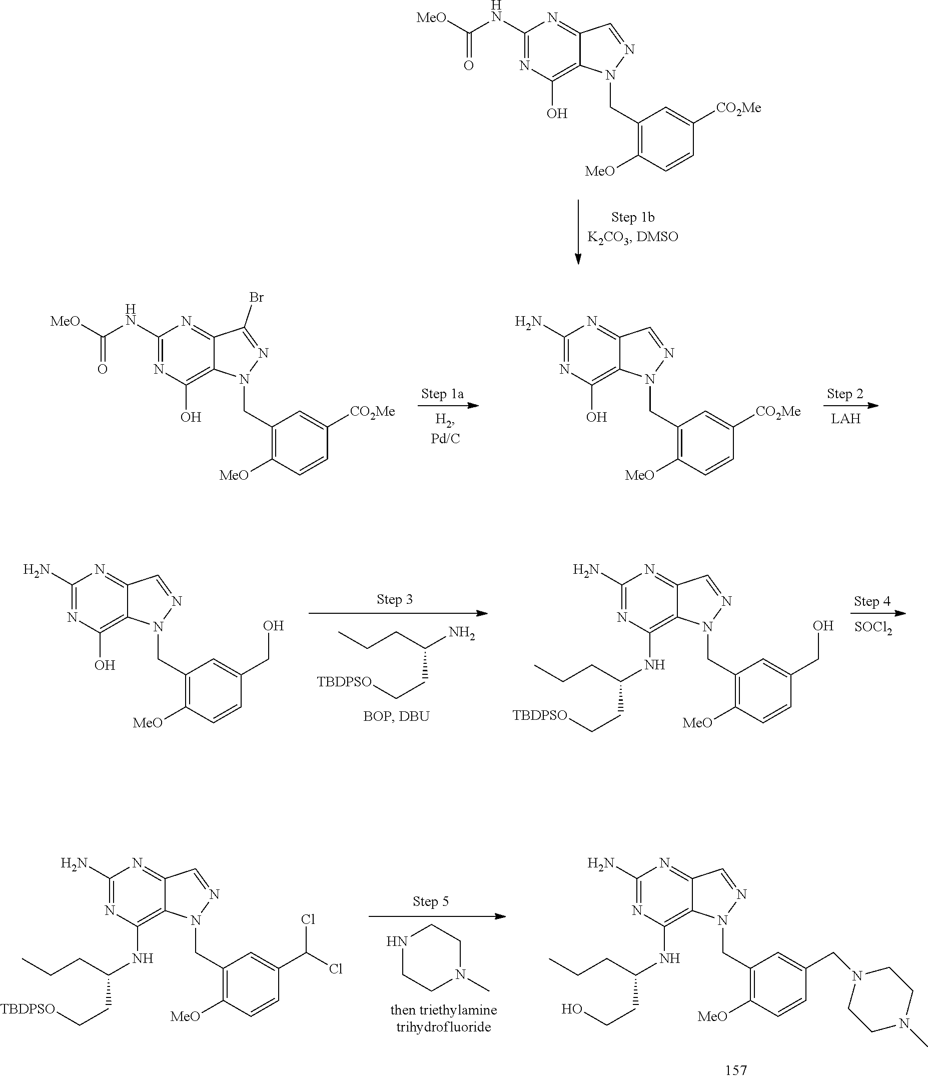

Step 1a. A mixture of methyl 3-((3-bromo-7-hydroxy-5-((methoxycarbonyl)amino)-1H-pyrazolo[4,3-d]pyrimidin-1-yl)methyl)-4-methoxybenzoate (1.1 g, 2.359 mmol) and Pd/C (0.500 g, 2.359 mmol, prepared in the previous patent) in DMSO (30 mL) and EtOH (10 mL) was stirred under H₂ at 80° C. for 3 days. The catalyst was filtered off, and the filtrate was concentrated. The crude product was purified on a silica gel column with 0-10% MeOH in CH₂Cl₂ to provide methyl 3-((5-amino-7-hydroxy-1H-pyrazolo[4,3-d]pyrimidin-1-yl)methyl)-4-methoxybenzoate as a white solid. LC-MS m/z 330.1 [M+H]⁺. ¹H NMR (400 MHz, DMSO-d6) δ 10.99 (s, 1H), 7.90 (dd, J=8.6, 2.2 Hz, 1H), 7.61 (s, 1H), 7.21-7.12 (m, 2H), 6.10 (s, 2H), 5.65 (s, 2H), 3.91 (s, 3H), 3.75 (s, 3H).

Step 1b. A mixture of methyl 3-((7-hydroxy-5-((methoxycarbonyl)amino)-1H-pyrazolo[4,3-d]pyrimidin-1-yl)methyl)-4-methoxybenzoate (0.1 g, 0.258 mmol, prepared by BBRC) and K₂CO₃ (0.107 g, 0.774 mmol) in DMSO (2 mL) was stirred at 80° C. for 90 min. After cooling, the reaction mixture was quenched by addition of water. The aqueous solution was extracted with EtOAc. The combined organic layers were dried, filtered, and concentrated. The crude product was purified on a silica gel column with 0-10% MeOH in CH₂Cl₂ to provide methyl 3-((5-amino-7-hydroxy-1H-pyrazolo[4,3-d]pyrimidin-1-yl)methyl)-4-methoxybenzoate as a white solid. LC-MS m/z 330.1 [M+H]⁺.

Step 2. A solution of methyl 3-((5-amino-7-hydroxy-1H-pyrazolo[4,3-d]pyrimidin-1-yl)methyl)-4-methoxybenzoate (0.274 g, 0.832 mmol) in THF (20 mL) was cooled to 0° C., and then treated with LiAlH₄ (2M in THF) (0.416 mL, 0.832 mmol) dropwise. LCMS after 1 h showed completion of reaction. The reaction was quenched by slow addition of methanol, and then stirred with Rochelle salt (1M, 10 mL) for 1 h. The aqueous solution was extracted with EtOAC. The combined organic layers were dried, filtered, and concentrated. The crude product was purified on a silica gel column with 0-10% MeOH in CH₂Cl₂ to provide 5-amino-1-(5-(hydroxymethyl)-2-methoxybenzyl)-1H-pyrazolo[4,3-d]pyrimidin-7-ol as a white solid.

LC-MS m/z 302.1 [M+H]⁺.

¹H NMR (400 MHz, DMSO-d6) δ 7.55 (s, 1H), 7.17 (dd, J=8.3, 2.1 Hz, 1H), 6.96 (d, J=8.4 Hz, 1H), 6.51 (d, J=2.1 Hz, 1H), 6.10 (s, 2H), 5.62 (s, 2H), 4.96 (t, J=5.8 Hz, 1H), 4.28 (d, J=5.4 Hz, 2H), 3.81 (s, 3H).

Step 3. A solution of 5-amino-1-(5-(hydroxymethyl)-2-methoxybenzyl)-1H-pyrazolo[4,3-d]pyrimidin-7-ol (0.13 g, 0.431 mmol), (S)-1-((tert-butyldiphenylsilyl)oxy)hexan-3-amine (0.307 g, 0.863 mmol) in DMSO (5 mL) was treated with BOP (0.382 g, 0.863 mmol) and DBU (0.260 mL, 1.726 mmol). The reaction mixture was heated at 60° C. overnight. Water was added to quench the reaction. The aqueous solution was extracted with EtOAc. The combined organic layers were dried, filter, and concentrated. The crude product was purified on a silica gel column with 0-10% MeOH in CH₂Cl₂ to provide (S)-(3-((5-amino-7-((1-((tert-butyldiphenylsilyl)oxy)hexan-3-yl)amino)-1H-pyrazolo[4,3-d]pyrimidin-1-yl)methyl)-4-methoxyphenyl)methanol as a white solid.

LC-MS m/z 639.3 [M+H]⁺.

Step 4. A mixture of (S)-(3-((5-amino-7-((1-((tert-butyldiphenylsilyl)oxy)hexan-3-yl)amino)-1H-pyrazolo[4,3-d]pyrimidin-1-yl)methyl)-4-methoxyphenyl)methanol (0.24 g, 0.376 mmol) and SOCl₂ (0.545 mL, 7.51 mmol) in THF (2 mL) was stirred at RT for 30 min. The solvent was evaporated off to afford (S)-N7-(1-((tert-butyldiphenylsilyl)oxy)hexan-3-yl)-1-(5-(chloromethyl)-2-methoxybenzyl)-1H-pyrazolo[4,3-d]pyrimidine-5,7-diamine as a white solid. LC-MS m/z 657.3 [M+H]⁺.

Step 5. (S)-3-((5-amino-1-(2-methoxy-5-((4-methylpiperazin-1-yl)methyl)benzyl)-1H-pyrazolo[4,3-d]pyrimidin-7-yl)amino)hexan-1-ol (Compound 157). A mixture of (S)-N7-(1-((tert-butyldiphenylsilyl)oxy)hexan-3-yl)-1-(5-(chloromethyl)-2-methoxybenzyl)-1H-pyrazolo-[4,3-d]pyrimidine-5,7-diamine (15 mg, 0.023 mmol) and 1-methylpiperazine (13.71 mg, 0.137 mmol) in DMF (0.5 mL) was stirred at RT for 2 h. Triethylamine trihydrofluoride (0.022 mL, 0.137 mmol) and DMSO (0.5 mL) were added. The reaction mixture was stirred at RT overnight. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS signals. Fractions containing Compound 157 were combined and dried via centrifugal evaporation.

The following compounds were analogously prepared per this Example: Compound 155, Compound 156, and Compound 158.

### Example I—Compound 164

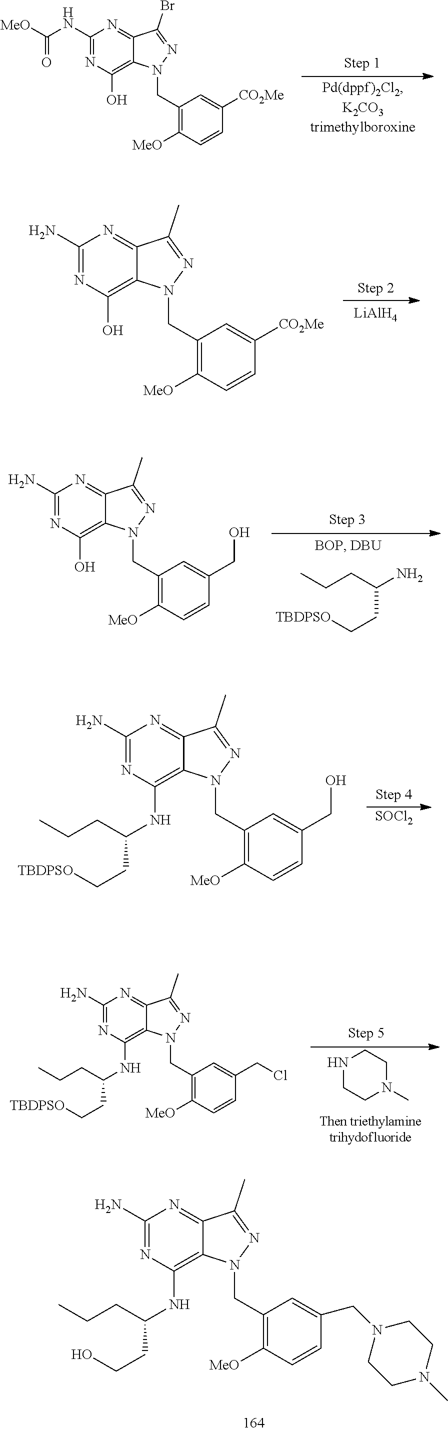

Step 1. A mixture of methyl 3-((3-bromo-7-hydroxy-5-((methoxycarbonyl)amino)-1H-pyrazolo[4,3-d]pyrimidin-1-yl)methyl)-4-methoxybenzoate (0.6 g, 1.287 mmol, prepared in the previous patent), Pd(dppf)₂Cl₂ (0.094 g, 0.129 mmol), K₂CO₃ (0.534 g, 3.86 mmol) and trimethyl-boroxine (0.899 mL, 6.43 mmol) was stirred at 120° C. overnight. After cooling, the reaction was quenched by addition of water. The aqueous solution was extracted with EtOAc. The combined organic layers were dried, filtered, and concentrated. The crude product was purified on a silica gel column with 0-10% MeOH in CH₂Cl₂ to provide methyl 3-((5-amino-7-hydroxy-3-methyl-1H-pyrazolo[4,3-d]pyrimidin-1-yl)methyl)-4-methoxybenzoate as a white solid.

LC-MS m/z 344.2 [M+H]⁺.

¹H NMR (500 MHz, DMSO-d6) δ 10.91 (s, 1H), 7.87 (dd, J=8.5, 2.0 Hz, 1H), 7.19-7.12 (m, 2H), 6.08 (s, 2H), 5.55 (s, 2H), 3.88 (s, 3H), 3.73 (s, 3H), 2.21 (s, 3H).

Step 2. A solution of methyl 3-((5-amino-7-hydroxy-3-methyl-1H-pyrazolo[4,3-d]pyrimidin-1-yl)methyl)-4-methoxybenzoate (0.12 g, 0.350 mmol) in THF (20 mL) was cooled to 0° C. and then treated with LiAlH₄ (2M in THF) (0.175 mL, 0.350 mmol) dropwise. LCMS after 2 h showed completion of reaction. The reaction was quenched by slow addition of methanol and then stirred with Rochelle salt (1M, 10 mL) for 1 h. The aqueous solution was extracted with EtOAC. The combined organic layers were dried, filtered, and concentrated. The crude product was purified on a silica gel column with 0-20% MeOH in CH₂Cl₂ to provide 5-amino-1-(5-(hydroxymethyl)-2-methoxybenzyl)-3-methyl-1H-pyrazolo[4,3-d]pyrimidin-7-ol as a white solid.

LC-MS m/z 316.2 [M+H]⁺.

¹H NMR (400 MHz, DMSO-d6) δ 7.16 (d, J=8.3 Hz, 1H), 6.96 (d, J=8.3 Hz, 1H), 6.54 (s, 1H), 6.09 (s, 2H), 5.54 (s, 2H), 4.96 (t, J=5.7 Hz, 1H), 4.28 (d, J=5.5 Hz, 2H), 3.80 (s, 3H), 2.22 (s, 3H).

Step 3. To a solution of 5-amino-1-(5-(hydroxymethyl)-2-methoxybenzyl)-3-methyl-1H-pyrazolo[4,3-d]pyrimidin-7-ol (74.8 mg, 0.237 mmol) and BOP (210 mg, 0.474 mmol) in DMSO (2 mL) was added a solution of (S)-1-((tert-butyldiphenylsilyl)oxy)hexan-3-amine (506 mg, 1.423 mmol) and DBU (0.143 mL, 0.949 mmol) in DMSO (2 mL). The reaction mixture was heated at 60° C. for 6 h. Water was added to quench the reaction. The aqueous solution was extracted with EtOAc. The combined organic layers were dried, filter, and concentrated. The crude product was purified on a silica gel column with 0-20% MeOH in CH₂Cl₂ to provide (S)-(3-((5-amino-7-((1-((tert-butyldiphenylsilyl)oxy)hexan-3-yl)amino)-3-methyl-1H-pyrazolo[4,3-d]pyrimidin-1-yl)methyl)-4-methoxyphenyl)methanol as a light yellow oil.

LC-MS m/z 653.5 [M+H]⁺.

Step 4. SOCl₂ (0.333 mL, 4.59 mmol) was added to a solution of (S)-(3-((5-amino-7-((1-((tert-butyldiphenylsilyl)oxy)hexan-3-yl)amino)-3-methyl-1H-pyrazolo[4,3-d]pyrimidin-1-yl)methyl)-4-methoxyphenyl)methanol (0.15 g, 0.230 mmol) in THF (2 mL) at RT. the reaction mixture was stirred at RT for 20 min. The solvent was evaporated off to afford (S)-N7-(1-((tert-butyldiphenylsilyl)oxy)hexan-3-yl)-1-(5-(chloromethyl)-2-methoxybenzyl)-3-methyl-1H-pyrazolo[4,3-d]pyrimidine-5,7-diamine as a white solid.

LC-MS m/z 671.5 [M+H]⁺.

Step 5. 1-methylpiperazine (17.90 mg, 0.179 mmol) was added to a solution of (S)-N7-(1-((tert-butyldiphenylsilyl)oxy)hexan-3-yl)-1-(5-(chloromethyl)-2-methoxybenzyl)-3-methyl-1H-pyrazolo[4,3-d]pyrimidine-5,7-diamine (20 mg, 0.030 mmol) in DMF (0.5 mL). The reaction mixture was stirred at RT for 3 h. Triethylamine trihydrofluoride (0.029 mL, 0.179 mmol) in DMSO (0.5 mL) was added. The reaction mixture was stirred at RT overnight. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 10% B, 10-50% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 164.

The following compounds were analogously prepared per this Example: Compound 159, Compound 161, and Compound 162.

### Example J—Compound 169

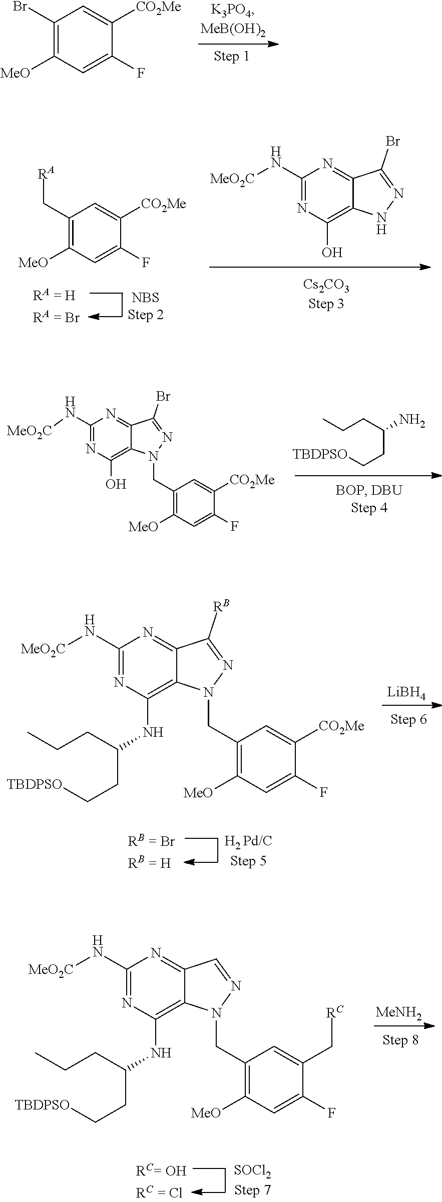

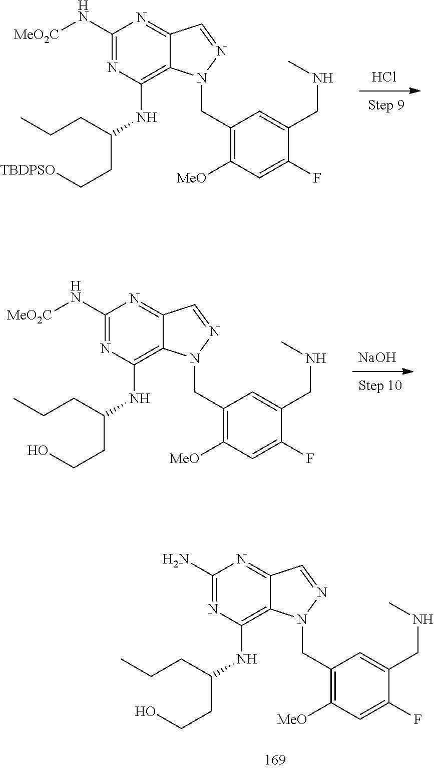

Step 1. A RT mixture of methyl 5-bromo-2-fluoro-4-methoxybenzoate (2.239 g, 8.51 mmol, prepared according to US 2015/0299104) and tribasic potassium phosphate (5.42 g, 25.5 mmol) in 1,4-dioxane (38.3 ml) and H₂O (4.26 ml) was sparged with N₂ for 30 min. Methyl-boronic acid (0.764 g, 12.77 mmol) and XPhos Pd G2 (0.167 g, 0.213 mmol) were added. The mixture was sparged with N₂ for 2 min and was stirred at 80° C. for 22 h. The reaction was cooled to RT, diluted with EtOAc (200 mL), washed with H₂O (200 mL) and saturated aqueous NaCl (200 mL), dried over Na₂SO₄, filtered, and concentrated in vacuo. The crude material was purified by flash chromatography (40 g silica gel; linear gradient 0-25% EtOAc-hexanes). The mixed fractions were concentrated and further purified by flash chromatography (40 g silica gel; linear gradient 0-25% EtOAc-hexanes). The products from both columns were combined to provide methyl 2-fluoro-4-methoxy-5-methylbenzoate (1.563 g, 93%).

LC-MS m/z 199 [M+H]⁺.

¹H NMR (400 MHz, DMSO-d₆) δ 7.67 (dd, J=8.6, 0.7 Hz, 1H), 6.95 (d, J=13.2 Hz, 1H), 3.87 (s, 3H), 3.80 (s, 3H), 2.13 (s, 3H).

Step 2. To a RT solution of methyl 2-fluoro-4-methoxy-5-methylbenzoate (1.563 g, 7.89 mmol) in CCl₄ (19.72 ml) was added N-bromosuccinimide (1.474 g, 8.28 mmol) and 2,2′-azobis(2-methylpropionitrile) (0.130 g, 0.789 mmol). The suspension was stirred at 75° C. for 20 h. The reaction was cooled to RT and filtered. The solids were washed with CCl₄ (2×2 mL). The combined filtrates were concentrated in vacuo. The crude material was purified by flash chromatography (40 g silica gel; linear gradient 0-25% EtOAc-hexanes). The mixed fractions were concentrated and further purified by flash chromatography (40 g silica gel; linear gradient 0-15% EtOAc-hexanes). The products from both columns were combined to provide methyl 5-(bromomethyl)-2-fluoro-4-methoxybenzoate (1.73 g, 79%).

LC-MS m/z 277/279 [M+H]⁺.

¹H NMR (400 MHz, DMSO-d₆) δ 8.00 (d, J=8.5 Hz, 1H), 7.09 (d, J=13.2 Hz, 1H), 4.67 (s, 2H), 3.95 (s, 3H), 3.82 (s, 3H).

Step 3. To a RT solution of methyl (3-bromo-7-hydroxy-1H-pyrazolo[4,3-d]pyrimidin-5-yl)carbamate (1.60 g, 5.55 mmol) (Scheme 2, compound 11, above) in DMF (27.8 ml) was added Cs₂CO₃ (5.43 g, 16.66 mmol). The reaction was stirred at 0° C. for 10 min, then methyl 5-(bromomethyl)-2-fluoro-4-methoxybenzoate (1.539 g, 5.55 mmol) was added. The reaction was stirred at 0° C. for 30 min, then the cooling bath was removed and it was stirred at RT for 1 h. The reaction was added to H₂O (150 mL), and the solids were collected by vacuum filtration and washed with H₂O (3×10 mL), MeOH (3×10 mL), and Et₂O (3×10 mL) to provide methyl 5-((3-bromo-7-hydroxy-5-((methoxycarbonyl)amino)-1H-pyrazolo[4,3-d]pyrimidin-1-yl)methyl)-2-fluoro-4-methoxybenzoate (1.191 g, 44%) as an off-white solid.

LC-MS m/z 484/486 [M+H]⁺.

¹H NMR (400 MHz, DMSO-d₆) δ 11.82-11.58 (m, 1H), 11.51-11.31 (m, 1H), 7.59 (d, J=8.3 Hz, 1H), 7.07 (d, J=13.1 Hz, 1H), 5.68 (s, 2H), 3.84 (s, 3H), 3.79 (s, 3H), 3.75 (s, 3H).

Step 4. To a RT suspension of methyl 5-((3-bromo-7-hydroxy-5-((methoxycarbonyl)-amino)-1H-pyrazolo[4,3-d]pyrimidin-1-yl)methyl)-2-fluoro-4-methoxybenzoate (569 mg, 1.175 mmol) in DMSO (7834 μl) was added (S)-1-((tert-butyldiphenylsilyl)oxy)hexan-3-amine, HCl (691 mg, 1.763 mmol) (US 2020/0038403 A1, FIG. 8, compound 71a) and (benzotriazol-1-yloxy)tris-(dimethylamino)phosphonium hexafluorophosphate (780 mg, 1.763 mmol), followed by 1,8-diazabicyclo[5.4.0]undec-7-ene (879 μl, 5.88 mmol). The reaction was stirred at RT for 4 h. Additional (benzotriazol-1-yloxy)tris(dimethylamino)phosphonium hexafluorophosphate (52 mg, 0.12 mmol) was added, and the reaction was stirred at RT for 19 h. The reaction was added to a stirred flask of H₂O (80 mL), and the insoluble material was collected by vacuum filtration and washed with H₂O (2×5 mL), and then it was dissolved in EtOAc (100 mL). The resulting solution was washed with saturated aqueous NaCl (100 mL), dried over Na₂SO₄, filtered, and concentrated in vacuo. The crude material was purified by flash chromatography (80 g silica gel; linear gradient 0-40% EtOAc-CH₂Cl₂). This material was further purified by flash chromatography (40 g silica gel; linear gradient 0-50% EtOAc-hexanes) to provide methyl (S)-5-((3-bromo-7-((1-((tert-butyldiphenylsilyl)oxy)hexan-3-yl)amino)-5-((methoxycarbonyl)amino)-1H-pyrazolo-[4,3-d]pyrimidin-1-yl)methyl)-2-fluoro-4-methoxybenzoate (455 mg, 47%) as a brown foam.

LC-MS m/z 821/823 [M+H]⁺.

¹H NMR (400 MHz, DMSO-d₆) δ 9.80 (s, 1H), 7.55 (dd, J=7.9, 1.5 Hz, 2H), 7.50-7.46 (m, 2H), 7.46-7.32 (m, 5H), 7.27-7.21 (m, 2H), 7.03 (d, J=13.1 Hz, 1H), 6.68 (br d, J=8.5 Hz, 1H), 5.77-5.69 (m, 1H), 5.67-5.59 (m, 1H), 4.70-4.60 (m, 1H), 3.74 (s, 6H), 3.65 (t, J=6.5 Hz, 2H), 3.58 (s, 3H), 1.91-1.83 (m, 2H), 1.61-1.43 (m, 2H), 1.27-1.13 (m, 2H), 0.91 (s, 9H), 0.80 (t, J=7.4 Hz, 3H).

Step 5. A RT solution of methyl (S)-5-((3-bromo-7-((1-((tert-butyldiphenylsilyl)oxy)-hexan-3-yl)amino)-5-((methoxycarbonyl)amino)-1H-pyrazolo[4,3-d]pyrimidin-1-yl)methyl)-2-fluoro-4-methoxybenzoate (0.455 g, 0.554 mmol) in EtOH (22.15 ml) was evacuated and then back-filled with N₂ (3×), and then palladium on carbon (10 wt % (dry basis), wet support) (0.088 g) was added. The mixture was evacuated and then back-filled with H₂, and stirred under an atmosphere of H₂ (balloon) for 2 h. The reaction mixture was purged with N₂ for 30 min, then it was filtered through CELITE™ under a blanket of N₂ and washed with EtOH (2×15 mL). The combined filtrates were concentrated in vacuo to provide methyl (S)-5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-2-fluoro-4-methoxybenzoate (423 mg, quant.) as a white foam.

LC-MS m/z 743 [M+H]⁺.

¹H NMR (400 MHz, DMSO-d₆) δ 8.03 (s, 1H), 7.56-7.49 (m, 3H), 7.50-7.46 (m, 2H), 7.43-7.32 (m, 4H), 7.29-7.24 (m, 2H), 7.04 (d, J=13.1 Hz, 1H), 5.85-5.78 (m, 1H), 5.73-5.66 (m, 1H), 4.68-4.58 (m, 1H), 3.76 (s, 3H), 3.77 (br s, 3H), 3.73 (s, 3H), 3.70-3.62 (m, 2H), 1.92 (br dd, J=5.3, 3.2 Hz, 2H), 1.67-1.51 (m, 2H), 1.29-1.14 (m, 2H), 0.91 (s, 9H), 0.82 (t, J=7.3 Hz, 3H).

Step 6. To a 0° C. solution of methyl (S)-5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-2-fluoro-4-methoxybenzoate (422 mg, 0.568 mmol) in a mixture of THF (5112 μl) and MeOH (568 μl) was added lithium borohydride (2 M solution in THF) (2840 μl, 5.68 mmol), dropwise. The reaction was stirred at RT for 17 h. Additional lithium borohydride (284 μL, 0.568 mmol) was added, and the reaction was stirred at RT for 30 min. Additional lithium borohydride (1.14 mL, 2.28 mmol) was added, and the reaction was stirred at RT for 30 min, and then at 40° C. for 5 h. The reaction was cooled to 0° C. and quenched by the slow addition of MeOH (2 mL). The mixture was stirred at RT for 15 min, and then it was diluted with H₂O (50 mL) and extracted with EtOAc (2×50 mL). The combined organic layers were washed with saturated aqueous NaCl (50 mL), dried over Na₂SO₄, filtered, and concentrated in vacuo. The crude material was purified by flash chromatography (40 g silica gel; linear gradient 0-100% EtOAc-CH₂Cl₂) to provide methyl (S)-(7-((1-((tert-butyldiphenylsilyl)oxy)hexan-3-yl)amino)-1-(4-fluoro-5-(hydroxymethyl)-2-methoxybenzyl)-1H-pyrazolo[4,3-d]pyrimidin-5-yl)carbamate (197.6 mg, 49%) as a white foam.

LC-MS m/z 715 [M+H]⁺.

¹H NMR (400 MHz, DMSO-d₆) δ 9.50 (s, 1H), 7.88 (s, 1H), 7.57-7.54 (m, 2H), 7.50-7.46 (m, 2H), 7.44-7.33 (m, 4H), 7.27-7.22 (m, 2H), 6.89 (d, J=12.1 Hz, 1H), 6.78 (d, J=8.5 Hz, 1H), 6.07 (d, J=8.5 Hz, 1H), 5.70-5.64 (m, 1H), 5.61-5.55 (m, 1H), 5.03 (t, J=5.6 Hz, 1H), 4.61-4.51 (m, 1H), 4.32-4.22 (m, 2H), 3.74 (s, 3H), 3.65-3.59 (m, 2H), 3.58 (s, 3H), 1.89-1.72 (m, 2H), 1.52-1.41 (m, 2H), 1.21-1.05 (m, 2H), 0.92 (s, 9H), 0.78 (t, J=7.3 Hz, 3H).

Step 7. To a 0° C. solution of thionyl chloride (104 μl, 1.427 mmol) in THF (2853 μl) was added a solution of methyl (S)-(7-((1-((tert-butyldiphenylsilyl)oxy)hexan-3-yl)amino)-1-(4-fluoro-5-(hydroxymethyl)-2-methoxybenzyl)-1H-pyrazolo[4,3-d]pyrimidin-5-yl)carbamate (204 mg, 0.285 mmol) in THF (2853 μl), dropwise. The reaction was stirred at RT for 20 min, and then it was concentrated in vacuo. The crude material was mixed with THF and concentrated in vacuo (2×) to provide crude methyl (S)-(7-((1-((tert-butyldiphenylsilyl)oxy)hexan-3-yl)amino)-1-(5-(chloromethyl)-4-fluoro-2-methoxybenzyl)-1H-pyrazolo[4,3-d]pyrimidin-5-yl)carbamate. This material was used without further purification.

LC-MS m/z 733 [M+H]⁺.

Step 8. A RT solution of methyl (S)-(7-((1-((tert-butyldiphenylsilyl)oxy)hexan-3-yl)amino)-1-(5-(chloromethyl)-4-fluoro-2-methoxybenzyl)-1H-pyrazolo[4,3-d]pyrimidin-5-yl)carbamate (0.042 g, 0.057 mmol) in MeCN (1.140 ml) was added to methylamine (2 M solution in THF) (0.086 ml, 0.171 mmol), and then N,N-diisopropylethylamine (0.060 ml, 0.342 mmol) was added. The reaction was stirred at 60° C. for 2 h then at 70° C. for 1 h. The reaction was cooled to RT and concentrated. The residue was taken up in EtOAc (2 mL) and washed with saturated aqueous NaHCO₃ (2 mL). The aqueous layer was extracted with EtOAc (2×2 mL). The combined organic layers were washed with saturated aqueous NaCl (2 mL), dried over Na₂SO₄, filtered, and concentrated in vacuo to provide crude methyl (S)-(7-((1-((tert-butyldiphenylsilyl)-oxy)hexan-3-yl)amino)-1-(4-fluoro-2-methoxy-5-((methylamino)methyl)benzyl)-1H-pyrazolo-[4,3-d]pyrimidin-5-yl)carbamate. This material was used without further purification.

LC-MS m/z 728 [M+H]⁺.

Step 9. To a RT solution of the crude material from Step 8 in 1,4-dioxane (570 μl) was added 4 N HCl in 1,4-dioxane (570 μl). The reaction was stirred at RT for 5 h, and concentrated. The residue was mixed with 1,4-dioxane (0.3 mL) and concentrated to provide crude methyl (S)-(1-(4-fluoro-2-methoxy-5-((methylamino)methyl)benzyl)-7-((1-hydroxyhexan-3-yl)amino)-1H-pyrazolo[4,3-d]pyrimidin-5-yl)carbamate. This material was used without further purification.

LC-MS m/z 490 [M+H]⁺.

Step 10. To a RT solution of the crude material from Step 9 in a mixture of 1,4-dioxane (570 μl) and MeOH (0.285 mL) was added 10 M aqueous NaOH (57.0 μl, 0.570 mmol). The reaction was stirred at 70° C. for 3 h. The reaction was cooled to RT and neutralized by the addition of acetic acid (32.6 μl, 0.570 mmol). The mixture was concentrated, and then it was dissolved in a mixture of H₂O (0.3 mL) and DMF (1.7 mL), filtered (0.45 μm nylon syringe filter), and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 minutes, then a 0-minute hold at 100% B; Flow Rate: 20 mL/min; Column Temperature: 25° C. Fraction collection was triggered by MS and UV signals. Fractions containing Compound 169 were combined and dried via centrifugal evaporation (10.4 mg, 41%).

These compounds were analogously prepared: Compound 165, Compound 166, Compound 167, Compound 168, Compound 171, Compound 172, Compound 173, Compound 175, and Compound 176. (In some cases, the following modifications were made to Step 8: additional equivalents of i-Pr₂NEt were added if the starting amine was a salt; the temperature of the reaction ranged from 60° C. to 80° C.).

### Example K—Compound 170

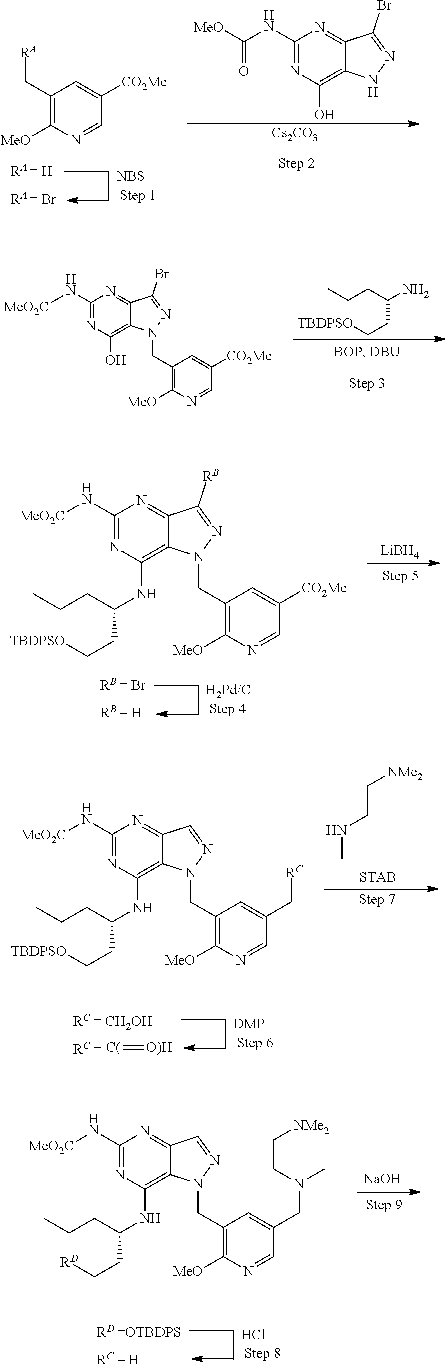

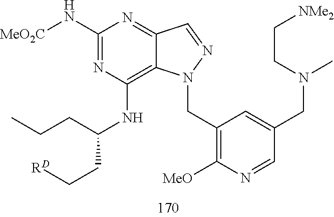

Step 1. A mixture of methyl 6-methoxy-5-methylnicotinate (491 mg, 2.71 mmol), NBS (627 mg, 3.52 mmol), and AlBN (111 mg, 0.677 mmol) in carbon tetrachloride (20 mL) was heated to 80° C. for 16 h. The reaction mixture was evaporated under reduced pressure and purified on a silica gel column with a gradient of 0% to 50% of ethyl acetate in hexanes to provide methyl 5-(bromomethyl)-6-methoxynicotinate (493 mg).

¹H NMR (400 MHz, CHLOROFORM-d) δ 8.84-8.74 (m, 1H), 8.28-8.18 (m, 1H), 4.54-4.46 (m, 2H), 4.15-4.07 (m, 3H), 3.98-3.89 (m, 3H)

Step 2. To a mixture of methyl 5-(bromomethyl)-6-methoxynicotinate (233 mg, 0.896 mmol) and methyl (3-bromo-7-hydroxy-1H-pyrazolo[4,3-d]pyrimidin-5-yl)carbamate (215 mg, 0.747 mmol) in DMF (5 mL) was added Cs₂CO₃ (730 mg, 2.240 mmol). After 16 h, the reaction was partitioned between ethyl acetate (50 mL)/LiCl (10% aqueous, 50 mL). The organic layer was dried with Na₂SO₄, filtered and concentrated under reduced pressure. The product was isolated by trituration with methanol to provide methyl 5-((3-bromo-7-hydroxy-5-((methoxycarbonyl)amino)-1H-pyrazolo[4,3-d]pyrimidin-1-yl)methyl)-6-methoxynicotinate (133 mg). This product was used without further purification.

LC-MS m/z 469.1 [M+H]⁺.

¹H NMR (400 MHz, DMSO-d₆) δ 11.69 (br s, 1H), 11.45 (br s, 1H), 8.72 (d, J=2.2 Hz, 1H), 7.85 (d, J=2.2 Hz, 1H), 5.73 (s, 2H), 3.99-3.92 (m, 3H), 3.83 (s, 3H), 3.76 (s, 3H).

Step 3. A solution of methyl 5-((3-bromo-7-hydroxy-5-((methoxycarbonyl)amino)-1H-pyrazolo[4,3-d]pyrimidin-1-yl)methyl)-6-methoxynicotinate (215 mg, 0.460 mmol), (S)-1-((tert-butyldiphenylsilyl)oxy)hexan-3-amine (245 mg, 0.690 mmol), BOP (305 mg, 0.690 mmol), and DBU (0.312 mL, 2.071 mmol) in DMSO (5 mL) was stirred for 16 h at RT. BOP (305 mg, 0.690 mmol) and DBU (0.312 mL, 2.071 mmol) were added. The reaction mixture was stirred 3 h at RT and partitioned between DCM (50 mL) and water (50 mL). The organic layer was dried with Na₂SO₄, filtered and concentrated under reduced pressure. The crude product was purified on a silica gel column with a gradient of 0% to 100% of ethyl acetate in hexanes to provide methyl (S)-5-((3-bromo-7-((1-((tert-butyldiphenylsilyl)oxy)hexan-3-yl)amino)-5-((methoxycarbonyl)-amino)-1H-pyrazolo[4,3-d]pyrimidin-1-yl)methyl)-6-methoxynicotinate (191 mg).

LC-MS m/z 804.4 [M+H]⁺.

Step 4. A suspension of methyl (S)-5-((3-bromo-7-((1-((tert-butyldiphenylsilyl)-oxy)hexan-3-yl)amino)-5-((methoxycarbonyl)amino)-1H-pyrazolo[4,3-d]pyrimidin-1-yl)methyl)-6-methoxynicotinate (191 mg, 0.237 mmol) and Pd-C (200 mg, 0.094 mmol) in MeOH (10 mL) was purged 3 times N₂ (evacuating in between) then purged three times with H₂ (evacuating in between). The mixture was stirred under hydrogen for 1 h. The reaction mixture was filtered through CELITE™ and evaporated under reduced pressure to provide methyl (S)-5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-6-methoxynicotinate (172 mg), used without further purification.

LC-MS m/z 726.3 [M+H]⁺.

Step 5. To a solution of methyl (S)-5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-6-methoxynicotinate (172 mg, 0.237 mmol) in a mixture of THF (3 mL) and methanol (0.600 mL) was added LiBH₄ (2M THF) (0.592 mL, 1.185 mmol). After 1 h, more LiBH₄ (2M THF) (0.592 mL, 1.185 mmol) was added. After 16 h the reaction was partitioned between ethyl acetate (50 mL) and 1% tartrate K/Na aqueous (10 mL). The organic layer was dried with Na₂SO₄, filtered and concentrated under reduced pressure. The crude product was purified on a silica gel column with a gradient of 0% to 100% of ethyl acetate in hexanes to provide methyl (S)-(7-((1-((tert-butyldiphenylsilyl)oxy)hexan-3-yl)amino)-1-((5-(hydroxymethyl)-2-methoxypyridin-3-yl)methyl)-1H-pyrazolo[4,3-d]pyrimidin-5-yl)carbamate (165 mg).

LC-MS m/z 698.5 [M+H]⁺.

Step 6. To a solution of methyl (S)-(7-((1-((tert-butyldiphenylsilyl)oxy)hexan-3-yl)amino)-1-((5-(hydroxymethyl)-2-methoxypyridin-3-yl)methyl)-1H-pyrazolo[4,3-d]pyrimidin-5-yl)carbamate (165 mg, 0.236 mmol) in DCM (10 mL) was added Dess-Martin periodinane (201 mg, 0.473 mmol). After 30 min the reaction was evaporated under reduced pressure and dried under high vacuum. The crude product was purified on a silica gel column with a gradient of 0% to 100% of EtOAc in hexanes to provide methyl (S)-(7-((1-((tert-butyldiphenylsilyl)oxy)hexan-3-yl)amino)-1-((5-formyl-2-methoxypyridin-3-yl)methyl)-1H-pyrazolo[4,3-d]pyrimidin-5-yl)carbamate (66 mg).

LC-MS m/z 696.5 [M+H]⁺.

Step 7. To a solution of methyl (S)-(7-((1-((tert-butyldiphenylsilyl)oxy)hexan-3-yl)amino)-1-((5-formyl-2-methoxypyridin-3-yl)methyl)-1H-pyrazolo[4,3-d]pyrimidin-5-yl)carbamate (33 mg, 0.047 mmol) and N1,N1,N2-trimethylethane-1,2-diamine (24.23 mg, 0.237 mmol) in DCM (3 mL) to give a solution to which was added sodium triacetoxy borohydride (70.4 mg, 0.332 mmol). After 2 h, 2 mL of saturated sodium carbonate was added. The resulting mixture was diluted with 20 mL of methanol. The organic layer was dried with Na₂SO₄, filtered and concentrated under reduced pressure to provide methyl (S)-(7-((1-((tert-butyldiphenylsilyl)oxy)hexan-3-yl)amino)-1-((5-(((2-(dimethylamino)ethyl)(methyl)amino)-methyl)-2-methoxypyridin-3-yl)methyl)-1H-pyrazolo[4,3-d]pyrimidin-5-yl)carbamate (37 mg). This product was used without further purification.

LC-MS m/z 782.5 [M+H]⁺.

Step 8. To a solution of methyl (S)-(7-((1-((tert-butyldiphenylsilyl)oxy)hexan-3-yl)amino)-1-((5-(((2-(dimethylamino)ethyl)(methyl)amino)methyl)-2-methoxypyridin-3-yl)methyl)-1H-pyrazolo[4,3-d]pyrimidin-5-yl)carbamate (0.037 g, 0.047 mmol) was added HCl (4N dioxane) (3 ml, 12.00 mmol). After 16 h the solvent was evaporated under reduced pressure and the residue dried under high vacuum to provide methyl (S)-(1-((5-(((2-(dimethylamino)-ethyl)(methyl)amino)methyl)-2-methoxypyridin-3-yl)methyl)-7-((1-hydroxyhexan-3-yl)amino)-1H-pyrazolo[4,3-d]pyrimidin-5-yl)carbamate hydrochloride (26 mg), used without further purification.

LC-MS m/z 544.3 [M+H]⁺.

Step 9. A solution of methyl (S)-(1-((5-(((2-(dimethylamino)ethyl)(methyl)amino)-methyl)-2-methoxypyridin-3-yl)methyl)-7-((1-hydroxyhexan-3-yl)amino)-1H-pyrazolo[4,3-d]pyrimidin-5-yl)carbamate (25.6 mg, 0.047 mmol) and NaOH (10N) (50 μl, 0.500 mmol) in dioxane (3 mL) was heated to 50° C. After 24 h, the solvent was evaporated under reduced pressure and the residue dried under high vacuum and diluted with 2 mL of DMF:HOAc (1:1). The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 7% B, 7-47% B over 20 minutes, then a 0-minute hold at 100% B; Flow Rate: 20 mL/min; Column Temperature: 25° C. Fraction collection was triggered by MS and UV signals. Fractions containing Compound 170 were combined and dried via centrifugal evaporation (11 mg).

### Example L—Compound 160

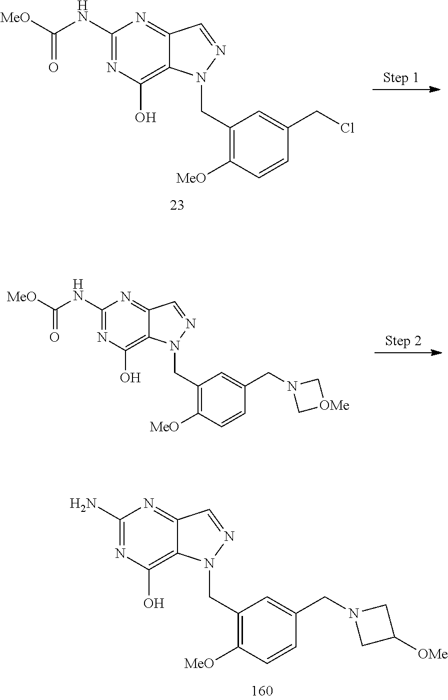

Step 1. To methyl (1-(5-(chloromethyl)-2-methoxybenzyl)-7-hydroxy-1H-pyrazolo[4,3-d]pyrimidin-5-yl)carbamate (Compound 23, 130 mg, 0.344 mmol) in DMF (3 mL) was added 3-methoxyazetidine (90 mg, 1.032 mmol) and DIPEA (0.240 mL, 1.376 mmol). The reaction stirred overnight at 25C. The solvent was removed via V-10 and the material purified on silica gel (dry load) 0-20% DCM-MeOH to afford methyl (7-hydroxy-1-(2-methoxy-5-((3-methoxyazetidin-1-yl)methyl)benzyl)-1H-pyrazolo[4,3-d]pyrimidin-5-yl)carbamate (130 mg, 0.303 mmol, 88% yield).

LC-MS m/z 429.4 [M+H]⁺.

Step 2. To methyl (7-hydroxy-1-(2-methoxy-5-((3-methoxyazetidin-1-yl)methyl)-benzyl)-1H-pyrazolo[4,3-d]pyrimidin-5-yl)carbamate (65 mg, 0.152 mmol) in DMSO (1.5 mL) was added (S)-3-amino-1-cyclopropylpropan-1-ol (34.9 mg, 0.303 mmol), DBU (0.091 mL, 0.607 mmol) and bop (134 mg, 0.303 mmol). The mixture stirred at 70C for 1 h. The mixture was treated with 5M NaOH (1 mL, 5.00 mmol) and heated at 70 degrees for 1 h. The crude product was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 minutes, then a 0-minute hold at 30 100% B; Flow Rate: 20 mL/min; Column Temperature: 25° C. Fraction collection was triggered by MS and UV signals. Fractions containing Compound 160 were combined and dried via centrifugal evaporation.

### Example M—Compound 163

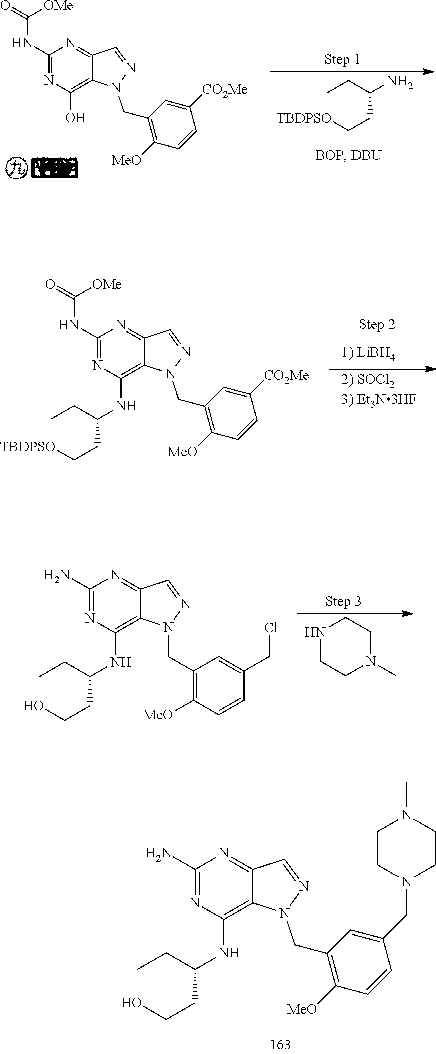

Step 1. A mixture of methyl 3-((7-hydroxy-5-((methoxycarbonyl)amino)-1H-pyrazolo[4,3-d]pyrimidin-1-yl)methyl)-4-methoxybenzoate (0.53 g, 1.368 mmol) in DMSO (8 mL) was treated with (S)-1-((tert-butyldiphenylsilyl)oxy)pentan-3-amine (1.402 g, 4.10 mmol), 2,3,4,6,7,8,9,10-octahydropyrimido[1,2-a]azepine (0.619 mL, 4.10 mmol) followed by ((1H-benzo[d][1,2,3]triazol-1-yl)oxy)tris(dimethylamino)phosphonium hexafluorophosphate(V) (1.210 g, 2.74 mmol) and stirred at RT for overnight. The reaction was diluted with EtOAc and washed with water. The solvent mixture was dried over Na₂SO₄. The solvent was removed and the material was purified on a 40 g COMBIFLASH silica gel column. 80% EtOAc/hexane fractions were concentrated to afford methyl (S)-3-((7-((1-((tert-butyldiphenylsilyl)oxy)pentan-3-yl)amino)-5-((methoxycarbonyl)amino)-1H-pyrazolo[4,3-d]pyrimidin-1-yl)methyl)-4-methoxybenzoate (0.32 g, 0.450 mmol, 32.9% yield) as a white solid.

LC/MS [M+H]=711.5.

Step 2. To a solution of methyl (S)-3-((7-((1-((tert-butyldiphenylsilyl)oxy)pentan-3-yl)amino)-5-((methoxycarbonyl)amino)-1H-pyrazolo[4,3-d]pyrimidin-1-yl)methyl)-4-methoxybenzoate (590 mg, 0.830 mmol) in THF (7469 μl) and MeOH (830 μl) was added lithium borohydride (2 M solution in THF) (4150 μl, 8.30 mmol), dropwise (gas evolution during addition). The reaction was stirred at RT for 30 min. The reaction was cooled to 0° C. and quenched by the addition of H₂O, causing precipitation of solids. The mixture was diluted with H₂O (50 mL) and extracted with EtOAc (2×50 mL) (layers were shaken until all the solids dissolved). The combined organic layers were washed with saturated aqueous NaCl (50 mL), dried over Na₂SO₄, filtered, and concentrated in vacuo. The crude material was purified by flash chromatography (loaded as a solution in CH₂Cl₂; 40 g silica gel; linear gradient 0-100% EtOAc-CH₂Cl₂ then 0-10% MeOH—CH₂Cl₂). Impurities eluted in the EtOAc gradient and product eluted in the MeOH gradient. The product fractions were concentrated to provide product as white solid. This was taken in THF (2 mL) and treated with thionyl chloride (0.062 mL, 0.843 mmol). The solvent was evaporated and re-dissolved in DMF (2 mL). Triethylamine trihydrofluoride (0.343 mL, 2.109 mmol) was added and stirred at RT for overnight at which LCMS shows completion of reaction. Purified on a COMBIFLASH 24 g column. 5% MeOH/DCM fractions provided methyl (S)-(1-(5-(chloromethyl)-2-methoxybenzyl)-7-((1-hydroxypentan-3-yl)amino)-1H-pyrazolo[4,3-d]pyrimidin-5-yl)carbamate (140 mg, 0.302 mmol, 36% yield) as thick oil. 15% MeOH/DCM fractions provided (S)-3-((5-amino-1-(5-(chloromethyl)-2-methoxybenzyl)-1H-pyrazolo[4,3-d]pyrimidin-7-yl)amino)pentan-1-ol (40 mg, 0.099 mmol, 12% yield).

LC/MS [M+H]=405.3.

Step 3. To a solution of (S)-3-((5-amino-1-(5-(chloromethyl)-2-methoxybenzyl)-1H-pyrazolo[4,3-d]pyrimidin-7-yl)amino)pentan-1-ol (0.099 mmol, 40 mg) in DMSO (1 mL) was added 1-methylpiperazine (0.494 mmol, 49.5 mg). The reaction mixture was heated at 80° C. for 1 h and purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing Compound 163 were combined and dried via centrifugal evaporation (white solid, 6.7 mg, 9% yield).

### Example N—Compound 178

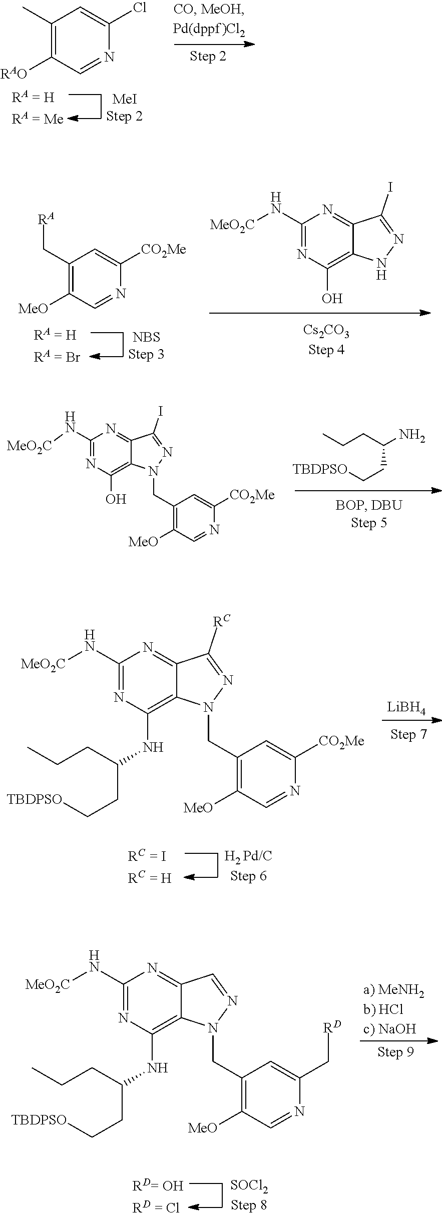

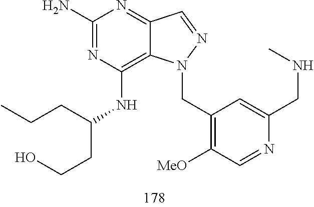

Step 1. A stirred solution of 2-chloro-5-methylpyridin-4-ol (5.00 g, 34.8 mmol) in DMF (50 mL) was cooled at 0° C. NaH (1.39 g, 34.8 mmol) was added. After 10 min, methyl iodide (2.61 mL, 41.8 mmol) was added. The reaction mixture was stirred at RT for 16 h and partitioned between water and ethyl acetate. The organic layer was washed with brine, dried over anhydrous Na₂SO₄, filtered and concentrated under vacuum to give crude product as a light yellow oil, which was purified using Combi Flash (silica gel 60-120 mesh; 15% ethyl acetate in petroleum ether as eluent). The fraction was concentrated using high vacuum at 50° C. to give 2-chloro-4-methoxy-5-methylpyridine (5.2 g, 32.7 mmol, 94% yield) as a yellow liquid. LC-MS [M+H]⁺ 158.2.

¹H NMR (400 MHz, DMSO-d) 6=8.00 (s, 1H), 7.33 (s, 1H), 3.89 (s, 3H), 2.17 (s, 3H).

Step 2. To a stirred solution of 2-chloro-4-methoxy-5-methylpyridine (5.750 g, 36.5 mmol) in DMF (100 mL) and methanol (100 mL), was added TEA (15.26 mL, 109 mmol). After purging with nitrogen for 5 min., PdCl₂(dppf)-CH₂Cl₂ adduct (5.96 g, 7.30 mmol) was added. The reaction mixture was stirred at 100° C. for 12 h under CO gas (10 kg pressure). The reaction mixture was filtered through a CELITE™ bed. The filtrate was washed with methanol and was concentrated under vacuum to give crude product as a light yellow oil. This was purified using Combi Flash (silica gel 60-120 mesh; 25% ethyl acetate in petroleum ether as eluent). The produce-containing fractions were concentrated using high vacuum at 50° C. to give methyl 4-methoxy-5-methylpicolinate (5.00 g, 27.6 mmol, 76% yield) as a brown solid.

LC-MS [M+H]⁺ 182.2.

¹H NMR (400 MHz, DMSO-d₆) δ=8.36 (s, 1H), 7.90 (s, 1H), 3.99 (s, 3H), 3.84 (s, 3H), 2.22 (s, 3H).

Step 3. To a solution of methyl 5-methoxy-4-methylpicolinate (5.00 g, 27.6 mmol) in carbon tetrachloride (100 mL), AlBN (0.906 g, 5.52 mmol) and NBS (5.89 g, 33.1 mmol) were added. The reaction mixture was stirred at 65° C. for 16 h and concentrated under vacuum. The residue was dissolved in ethyl acetate and partitioned between water and ethyl acetate. The organic layer was washed with brine, dried over anhydrous Na₂SO₄, filtered and concentrated under vacuum to give crude product as a light yellow oil, which was purified using Combi Flash (silica gel 60-120 mesh; 25% ethyl acetate in petroleum ether as eluent). The product containing fractions were concentrated using high vacuum at 50° C. to give methyl 4-(bromomethyl)-5-methoxypicolinate (5.1 g, 14.51 mmol, 52.6% yield) as a light yellow solid.

LC-MS [M+H]⁺: 260.1.

¹H NMR (400 MHz, DMSO-d₆) δ=8.59 (s, 1H), 8.13 (s, 1H), 4.66 (s, 2H), 4.03 (s, 3H), 3.86 (s, 3H).

Step 4. To a stirred solution of methyl (7-hydroxy-3-iodo-1H-pyrazolo[4,3-d]pyrimidin-5-yl)carbamate (1.600 g, 4.78 mmol) in DMF (20 mL), Cs₂CO₃ (3.11 g, 9.55 mmol) and methyl 4-(bromomethyl)-5-methoxypicolinate (1.242 g, 4.78 mmol) were added. The reaction mixture was stirred at 0° C. for 1 h. The reaction mixture was partitioned between water and ethyl acetate. The organic layer was washed with brine, dried over anhydrous Na₂SO₄, filtered and concentrated under vacuum to give crude product as a light yellow solid, which was purified using Combi Flash (silica gel 60-120 mesh; 10% ethyl acetate in chloroform as eluent). The product-containing fractions were concentrated using high vacuum at 50° C. to give methyl 4-((7-hydroxy-3-iodo-5-((methoxycarbonyl)amino)-1H-pyrazolo[4,3-d]pyrimidin-1-yl)methyl)-5-methoxypicolinate (1.100 g, 1.968 mmol, 41.2% yield) as an off-white solid.

LC-MS m/z 515.2 [M+H]⁺.

¹H NMR (400 MHz, DMSO-d₆) δ=11.71 (s, 1H), 11.40 (s, 1H) 8.51 (s, 1H), 7.43 (s, 1H), 5.73 (s, 2H), 4.04 (s, 3H), 3.80 (s, 3H), 3.73 (s, 3H).

Step 5. To a stirred solution of methyl 4-((7-hydroxy-3-iodo-5-((methoxycarbonyl)-amino)-1H-pyrazolo[4,3-d]pyrimidin-1-yl)methyl)-5-methoxypicolinate (1.100 g, 2.139 mmol) in DMSO (10 mL), DBU (0.967 mL, 6.42 mmol), BOP (1.419 g, 3.21 mmol) and (S)-1-((tert-butyldiphenylsilyl)oxy)hexan-3-amine (0.761 g, 2.139 mmol) were sequentially added. The reaction mixture was stirred at 45° C. for 4 h and then partitioned between water and ethyl acetate. The organic layer was washed with brine, dried over anhydrous Na₂SO₄, filtered and concentrated under vacuum to give crude product as a light yellow oil, which was purified using Combi Flash (silica gel 60-120 mesh; 25% ethyl acetate in chloroform as eluent). The fraction was concentrated using high vacuum at 50° C. to give methyl (S)-4-((7-((1-((tert-butyldiphenyl-silyl)oxy)hexan-3-yl)amino)-3-iodo-5-((methoxycarbonyl)amino)-1H-pyrazolo[4,3-d]pyrimidin-1-yl)methyl)-5-methoxypicolinate (1.10 g, 1.188 mmol, 55.5% yield) as a yellow solid.

LC-MS m/z 852.8 [M+H]⁺.

Step 6. To a stirred solution of methyl (S)-4-((7-((1-((tert-butyldiphenylsilyl)oxy)-hexan-3-yl)amino)-3-iodo-5-((methoxycarbonyl)amino)-1H-pyrazolo[4,3-d]pyrimidin-1-yl)methyl)-5-methoxypicolinate (1.30 g, 1.526 mmol) in methanol (15 mL), was added 10% palladium on carbon (0.812 g, 0.763 mmol). The reaction mixture was stirred at RT under H₂ for 14 h. The mixture was filtered through a CELITE™ bed. The filtrate was washed with methanol and DCM (400 mL) and was concentrated under vacuum 50° C. to give methyl (S)-4-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxypicolinate (1.050 g, 1.418 mmol, 93% yield) as a brown solid.

LC-MS m/z 726.3 [M+H]⁺.

Step 7. To a stirred solution of methyl (S)-4-((7-((1-((tert-butyldiphenylsilyl)oxy)-hexan-3-yl)amino)-5-((methoxycarbonyl)amino)-1H-pyrazolo[4,3-d]pyrimidin-1-yl)methyl)-5-methoxypicolinate (1.00 g, 1.378 mmol) in THF (10 mL):methanol (3 mL) at 0° C. LiBH₄ (10.33 mL, 20.66 mmol) was added. The reaction mixture was stirred at 45° C. for 16 h and quenched with ammonium chloride solution and extracted with ethyl acetate. The organic layer was washed with brine, dried over anhydrous Na₂SO₄, filtered and concentrated under vacuum to give crude product as an off-white solid. The crude product was purified using Combi Flash (silica gel 60-120 mesh; 5% methanol in chloroform as eluent). The fraction was concentrated using high vacuum at 50° C. to give methyl (S)-(7-((1-((tert-butyldiphenylsilyl)oxy)hexan-3-yl)amino)-1-((2-(hydroxymethyl)-5-methoxypyridin-4-yl)methyl)-1H-pyrazolo[4,3-d]pyrimidin-5-yl)carbamate (0.170 g, 0.173 mmol, 12.55% yield) as an off white solid.

LC-MS m/z 698.3 [M+H]⁺.

Step 8. To a stirred solution of methyl (S)-(7-((1-((tert-butyldiphenylsilyl)oxy)-hexan-3-yl)amino)-1-((2-(hydroxymethyl)-5-methoxypyridin-4-yl)methyl)-1H-pyrazolo[4,3-d]pyrimidin-5-yl)carbamate (0.200 g, 0.287 mmol) in THF (3 mL), thionyl chloride (0.105 mL, 1.433 mmol) was added at 0° C. The reaction mixture was stirred at 0° C. for 1 h. The reaction mixture was concentrated to give methyl (S)-(7-((1-((tert-butyldiphenylsilyl)oxy)hexan-3-yl)amino)-1-((2-(chloromethyl)-5-methoxypyridin-4-yl)methyl)-1H-pyrazolo[4,3-d]pyrimidin-5-yl)carbamate (0.226 g, 0.271 mmol, 95% yield) as a yellow oil.

LC-MS m/z 718.2 [M+H]⁺.

Step 9. To a stirred solution of methyl (S)-(7-((1-((tert-butyldiphenylsilyl)oxy)hexan-3-yl)amino)-1-((2-(chloromethyl)-5-methoxypyridin-4-yl)methyl)-1H-pyrazolo[4,3-d]pyrimidin-5-yl)carbamate (0.112 g, 0.156 mmol) in DMF (2 mL), methylamine HCl (0.021 g, 0.313 mmol) and K₂CO₃ (0.065 g, 0.469 mmol) were added. The reaction mixture was stirred at 50° C. for 14 h. The reaction mixture was concentrated in vacuo. The residue was dissolved in methanol (2 mL). HCl (5.21 μl, 0.172 mmol) in water (1 mL) was added. The reaction mixture was stirred at RT and concentrated in vacuo. The crude was taken in 1,4-dioxane (1 mL), to which NaOH (0.044 g, 1.100 mmol) in water (1 mL) was added. The reaction mixture was stirred at 70° C. for 3 h. The reaction mixture was partitioned between water and ethyl acetate. The organic layer was washed with brine solution, dried over anhydrous Na₂SO₄, filtered and concentrated under vacuum to give crude product as a light brown oil. The crude product was purified by preparative LC/MS (Column: Waters XBridge C18, 150 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 10% B, 10-45% B over 25 minutes, then a 5-minute hold at 100% B; Flow Rate: 15 mL/min; column temperature: 25° C. to afford Compound 178 (0.04 g, 2.4% yield).

Compound 177 was analogously prepared.

### Example O—Compound 174

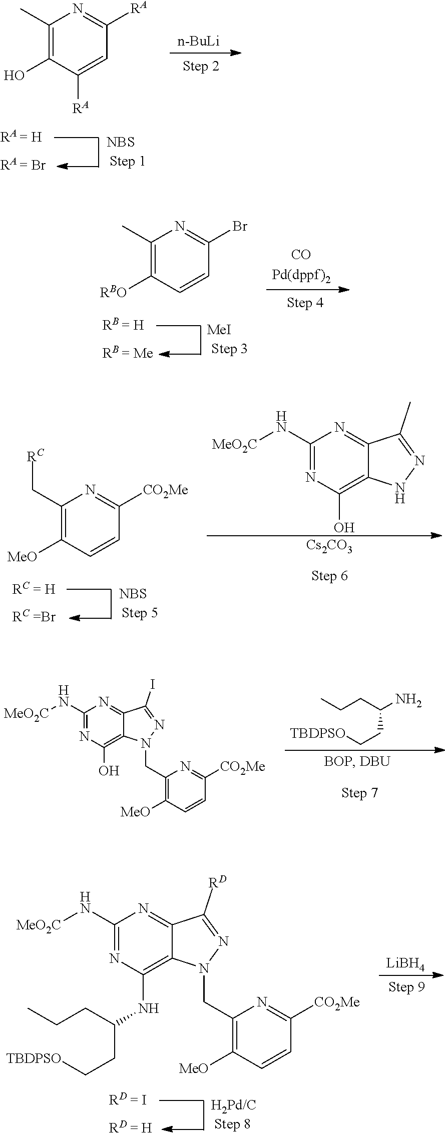

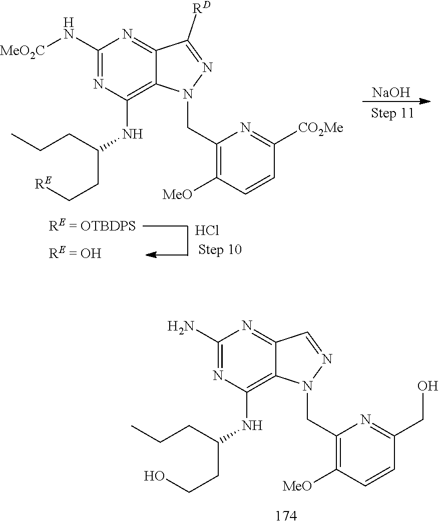

Step 1. To a stirred solution of 2-methylpyridin-3-ol (10.0 g, 92 mmol) in acetonitrile (150.0 mL), a solution of NBS (33.4 g, 188 mmol) in acetonitrile (350.0 mL) was added slowly over 1 h. The reaction mixture was stirred at 85° C. for 2 h. The reaction mixture was concentrated under reduced pressure to afford crude product, which was absorbed on silica gel and purified by ISCO COMBIFLASH™ chromatography by eluting with 0-100% ethyl acetate in chloroform to afford 4,6-dibromo-2-methylpyridin-3-ol (11.0 g, 39.6 mmol, 43.2% yield) as a light yellow solid.

LC-MS m/z 268.0 [M+H]⁺.

¹H NMR (300 MHz, DMSO-d₆) δ=9.98 (s, 1H), 7.70 (s, 1H), 2.41 (s, 3H).

Step 2. To a stirred solution of 4,6-dibromo-2-methylpyridin-3-ol (10.0 g, 37.5 mmol) in THF (150.0 mL), n-BuLi (31.5 mL, 79 mmol) was added at −78° C. The reaction mixture was stirred at same temperature for 3 h. To this mixture H₂O (30.0 mL, 1665 mmol) followed by addition of 1.5 N HCl solution (30.0 mL) at same temperature. The reaction mixture was stirred at same temperature for 10 min, diluted with saturated ammonium chloride solution and extracted with DCM. The organic layer was washed with brine and dried over Na₂SO₄, filtered and concentrated under reduced pressure to afford 6-bromo-2-methylpyridin-3-ol (5.1 g, 25.5 mmol, 68.1% yield) as a light brown solid.

LC-MS m/z 188.1 [M]⁺.

¹H NMR (300 MHz, DMSO-d₆) d=10.10 (br s, 1H), 7.24 (d, J=8.7 Hz, 1H), 7.08 (d, J=8.3 Hz, 1H), 2.34-2.23 (m, 3H).

Step 3. To a stirred solution of 6-bromo-2-methylpyridin-3-ol (4.0 g, 21.27 mmol) in acetonitrile (40.0 mL), Cs₂CO₃ (20.79 g, 63.8 mmol) was added. To this mixture Mel (1.995 mL, 31.9 mmol) was added. The reaction mixture was stirred at 50° C. for 16 h. The reaction mixture was partitioned between EtOAc and water. The organic layer was washed with brine solution and dried over Na₂SO₄, filtered and concentrated under reduced pressure to afford crude compound. The crude compound was rinsed with petroleum ether, the filtrate was concentrated under reduced pressure to afford 6-bromo-3-methoxy-2-methylpyridine (4.0 g, 18.81 mmol, 88% yield) as a brown solid.

LC-MS m/z 202.0 [M+H]⁺.

¹H NMR (300 MHz, CHLOROFORM-d) 6=7.23-7.14 (m, 1H), 6.90 (d, J=8.7 Hz, 1H), 3.75 (s, 3H), 2.37 (s, 3H).

Step 4. To a stirred solution of 6-bromo-3-methoxy-2-methylpyridine (4.0 g, 19.80 mmol) in DMF (40.0 mL): MeOH (40.0 mL), TEA (8.28 mL, 59.4 mmol), PdCl₂(dppf)-CH₂Cl₂ (3.23 g, 3.96 mmol) were added under nitrogen purging. The reaction mixture was stirred at 100° C. under CO gas (10 bar pressure) in an autoclave for 16 h. The reaction mixture was concentrated under reduced pressure to afford a residue. The residue was diluted with DCM and then filtered through a CELITE™ bed and washed with excess of DCM. The filtrate was concentrated under reduced pressure to afford crude compound. The crude compound was purified by ISCO Combiflash chromatography by eluting with 0-100% ethyl acetate in pet.ether to afford methyl 5-methoxy-6-methylpicolinate (2.62 g, 14.32 mmol, 72.3% yield) as a light brown solid.

LC-MS m/z 182.0 [M+H]⁺.

¹H NMR (300 MHz, DMSO-d₆) δ=7.98-7.91 (m, 1H), 7.49-7.40 (m, 1H), 3.92-3.87 (m, 3H), 3.86-3.80 (m, 3H), 2.42-2.36 (m, 3H).

Step 5. To a stirred solution of methyl 5-methoxy-6-methylpicolinate (2.5 g, 13.80 mmol) in chloroform (25.0 mL), NBS (2.95 g, 16.56 mmol) and AlBN (0.453 g, 2.76 mmol) were added. The reaction mixture was stirred at 65° C. for 16 h. The reaction mixture was filtered through a CELITE™ bed and washed with excess of DCM and the filtrate was concentrated under reduced pressure to afford crude compound. The crude compound was purified by ISCO Combiflash chromatography by eluting with 0-100% ethyl acetate in pet.ether to afford light brown solid, which was stirred in water for 15 minutes followed by filtering the solid and drying under vacuum to afford methyl 6-(bromomethyl)-5-methoxypicolinate (1.6 g, 5.84 mmol, 42.4% yield) as a light brown solid.

LC-MS m/z 262.0 [M+H]⁺.

¹H NMR (300 MHz, DMSO-d₆) δ=11.17-10.94 (m, 1H), 8.08 (d, J=8.7 Hz, 1H), 7.66-7.57 (m, 1H), 4.73-4.58 (m, 2H), 3.99-3.97 (m, 3H), 3.87-3.84 (m, 3H), 2.57-2.56 (m, 1H), 2.57 (s, 5H).

Step 6. To a stirred solution of methyl (7-hydroxy-3-iodo-1H-pyrazolo[4,3-d]pyrimidin-5-yl)carbamate (2.0 g, 5.97 mmol) in DMF (20.0 mL), Cs₂CO₃ (3.89 g, 11.94 mmol) was added. To this mixture methyl 6-(bromomethyl)-5-methoxypicolinate (1.552 g, 5.97 mmol) was added at 0° C. The reaction mixture was stirred at 0° C. for 1 h. The reaction mixture was partitioned between EtOAc and water. The organic layer was washed with brine solution and dried over Na₂SO₄, filtered and concentrated under reduced pressure to afford crude compound. The crude compound was purified by ISCO combiflash chromatography by eluting with 0-100% ethyl acetate in chloroform to afford methyl 6-((7-hydroxy-3-iodo-5-((methoxycarbonyl)amino)-1H-pyrazolo[4,3-d]pyrimidin-1-yl)methyl)-5-methoxypicolinate (1.08 g, 1.764 mmol, 29.6% yield) as a light brown solid.

LC-MS m/z 515.0 [M+H]⁺.

Step 7. To a stirred solution of methyl 6-((7-hydroxy-3-iodo-5-((methoxy-carbonyl)amino)-1H-pyrazolo[4,3-d]pyrimidin-1-yl)methyl)-5-methoxypicolinate (0.32 g, 0.622 mmol) in DMSO (3.0 mL), DBU (0.281 mL, 1.867 mmol), BOP (0.413 g, 0.933 mmol) and (S)-1-((tert-butyldiphenylsilyl)oxy)hexan-3-amine (0.266 g, 0.747 mmol) were added. The reaction mixture was stirred at 45° C. for 3 h. The reaction mixture was treated with water. The precipitate was collected and dried under vacuum to afford crude compound. The crude compound was purified by ISCO combiflash chromatography by eluting with 0-100% ethyl acetate in pet.ether to afford methyl (S)-6-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-5-methoxypicolinate (0.189 g, 0.220 mmol, 35.3% yield) as a light brown solid.

LC-MS m/z 852.2 [M+H]⁺.

Step 8. To a stirred solution of methyl (S)-6-((7-((1-((tert-butyldiphenylsilyl)oxy)-hexan-3-yl)amino)-3-iodo-5-((methoxycarbonyl)amino)-1H-pyrazolo[4,3-d]pyrimidin-1-yl)methyl)-5-methoxypicolinate (0.16 g, 0.188 mmol) in MeOH (5.0 mL), Pd—C(0.100 g, 0.094 mmol) was added. The reaction mixture was stirred at RT under hydrogen gas (bladder) for 4 h. The reaction mixture was filtered through a CELITE™ bed and washed with excess of methanol DCM (1:1) and the filtrate was concentrated under reduced pressure to afford crude compound. The crude compound was triturated with diethyl ether and petroleum ether, the solid was dried under vacuum to afford methyl (S)-6-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxypicolinate (0.118 g, 0.135 mmol, 71.8% yield) as a light brown solid.

LC-MS m/z 726.3 [M+H]⁺.

Step 9. To a stirred solution of methyl (S)-6-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-5-methoxypicolinate (0.1 g, 0.138 mmol) in THF (3.5 mL): MeOH (1.5 mL), LiBH₄ (2M in THF) (0.344 mL, 0.689 mmol) was added. The reaction mixture was stirred at 45° C. for 16 h. To this mixture LiBH₄ (2M in THF) (0.689 mL, 1.378 mmol) was added. The reaction mixture was stirred at 45° C. for 18 h and quenched with saturated aq. NH₄Cl solution. The organic layer was separated, washed with brine, and dried over Na₂SO₄, filtered and concentrated under reduced pressure to afford methyl (S)-(7-((1-((tert-butyldiphenylsilyl)oxy)hexan-3-yl)amino)-1-((6-(hydroxymethyl)-3-methoxypyridin-2-yl)methyl)-1H-pyrazolo[4,3-d]pyrimidin-5-yl)carbamate (0.11 g, 0.128 mmol, 93% yield) as an off-white semi solid.

LC-MS m/z 698.3 [M+H]⁺

Step 10. To a stirred solution of methyl (S)-(7-((1-((tert-butyldiphenylsilyl)-oxy)hexan-3-yl)amino)-1-((6-(hydroxymethyl)-3-methoxypyridin-2-yl)methyl)-1H-pyrazolo[4,3-d]pyrimidin-5-yl)carbamate (0.1 g, 0.143 mmol) in MeOH (1.5 mL), aqueous HCl (0.1 mL, 1.152 mmol) was added at 0° C. The reaction mixture was stirred at RT for 2 h. The reaction mixture was concentrated completely under reduced pressure and co-distilled with DCM to afford a crude compound. The crude compound was triturated with diethyl ether and pet.ether, the solid was dried under vacuum to afford methyl (S)-(7-((1-hydroxyhexan-3-yl)amino)-1-((6-(hydroxymethyl)-3-methoxypyridin-2-yl)methyl)-1H-pyrazolo[4,3-d]pyrimidin-5-yl)carbamate, HCl (85 mg, 0.141 mmol, 98% yield) as a light green semi solid.

LC-MS m/z 460.2 [M+H]⁺.

Step 11. To a stirred solution of methyl (S)-(7-((1-hydroxyhexan-3-yl)amino)-1-((6-(hydroxymethyl)-3-methoxypyridin-2-yl)methyl)-1H-pyrazolo[4,3-d]pyrimidin-5-yl)carbamate, HCl (80 mg, 0.161 mmol) in dioxane (1.0 mL): water (1.0 mL), NaOH (32.3 mg, 0.807 mmol) was added. The reaction mixture was stirred at 70° C. for 90 minutes. The organic layer was separated and concentrated under reduced pressure to afford crude compound. The crude product was purified by reverse phase preparative HPLC (Column: Waters XBridge C18, 150 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 7% B, 7-25% B over 20 minutes, then a 5-minute hold at 100% B; Flow Rate: 15 mL/min; Column Temperature: 25° C.) to afford Compound 174 (26.4 mg, 0.064 mmol, 40.0% yield).

### Example P—Compound 179

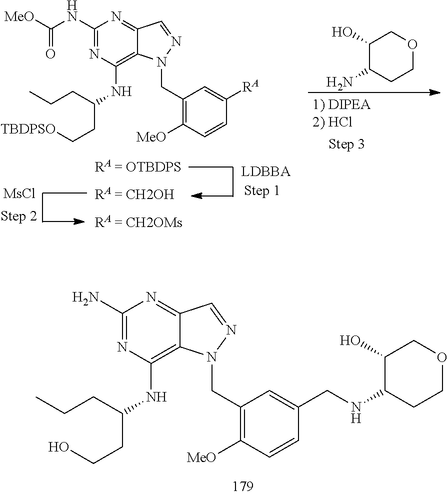

Step 1. Lithium diisobutyl-tert-butoxyaluminum hydride solution, 0.25 M in THF/hexanes (50 mL, 12.50 mmol) was added to a solution of methyl (S)-3-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxybenzoate (1.87 g, 2.58 mmol) in THF (25.8 mL) at 0° C. over 5 min. The reaction stirred at 25° C. overnight (3 h, 98% conversion). The solution was diluted with cold water and extracted with AcOEt 3 times. Finally, the organic layer was dried over Na₂SO₄ and evaporated under vacuum. The material was purified on silica gel (hexane-EtOAc 0-100%) to afford methyl (S)-(7-((1-((tert-butyldiphenylsilyl)oxy)hexan-3-yl)amino)-1-(5-(hydroxymethyl)-2-methoxybenzyl)-1H-pyrazolo[4,3-d]pyrimidin-5-yl)carbamate (1.56 g, 2.238 mmol, 87% yield).

LC-MS m/z 697.5[M+H]⁺.

Step 2. Methyl (S)-(7-((1-((tert-butyldiphenylsilyl) oxy) hexan-3-yl) amino)-1-(5-(hydroxymethyl)-2-methoxybenzyl)-1H-pyrazolo [4, 3-d] pyrimidin-5-yl) carbamate (0.51 g, 0.732 mmol) was dissolved in anhydrous CH₂Cl₂ (5 mL) in a 25 mL round bottom flask, to give a clear solution at 25° C. After the solution was cooled to 0° C., Et₃N (0.306 ml, 2.195 mmol) and Ms-Cl (0.114 ml, 1.464 mmol) were added. The reaction was complete after 15 min and was quenched with ice water and DCM. The organic layer was washed with brine and dried over Na₂SO₄. The solution was concentrated to give methyl (S)-(7-((1-((tert-butyldiphenylsilyl)-oxy)hexan-3-yl)amino)-1-(2-methoxy-5-(methoxymethyl)benzyl)-1H-pyrazolo[4,3-d]pyrimidin-5-yl)carbamate (0.35 g, 67.3% yield). The material was used without purification.

Step 3. (3S,4S)-4-Aminotetrahydro-2H-pyran-3-ol hydrochloride (70 mg, 0.456 mmol) and DIPEA (0.073 mL, 0.418 mmol) were added to (S)-3-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-4-methoxybenzyl methanesulfonate (108 mg, 0.139 mmol) in DMF (1 mL). The reaction was stirred for 12 h at 25° C. LC/MS confirmed formation of a first intermediate methyl (7-(((S)-1-((tert-butyldiphenylsilyl)oxy)hexan-3-yl)amino)-1-(5-((((3S,4S)-3-hydroxytetrahydro-2H-pyran-4-yl)amino)methyl)-2-methoxybenzyl)-1H-pyrazolo[4,3-d]pyrimidin-5-yl)carbamate. HCl in 1,4-dioxane (3 mL, 12.00 mmol) was added to the reaction mixture, followed by stirring 2 h at 25° C. The solvent was removed and the LC/MS confirmed formation of a second intermediate methyl (7-(((S)-1-hydroxyhexan-3-yl)amino)-1-(5-((((3S,4S)-3-hydroxytetrahydro-2H-pyran-4-yl)amino)methyl)-2-methoxybenzyl)-1H-pyrazolo[4,3-d]pyrimidin-5-yl)carbamate. The solvents were removed and the residue was diluted with 5M NaOH in MeOH, followed by stirring at 80° C. for 1 h. The LC/MS confirmed the desired material and the solvents were removed.

The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation.

The material was further purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation.

The material was still further purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 1% B, 1-41% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford compound 179 (15.9 mg, 0.031 mmol, 22.38% yield).

The following compounds were analogously prepared: Compound 180, Compound 181, Compound 182, Compound 183, and Compound 184.

### Example Q—Starting Materials and Intermediates

The Charts below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

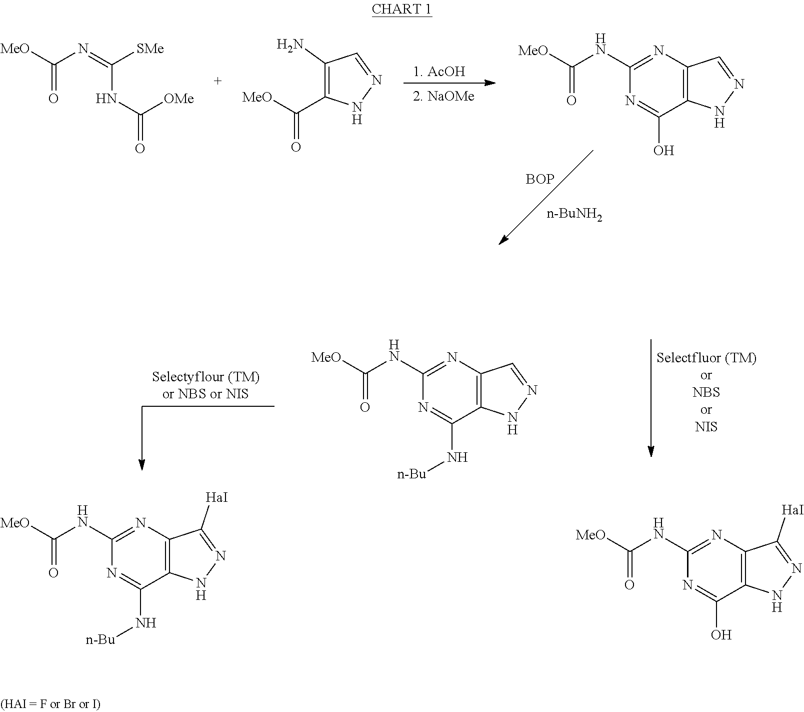

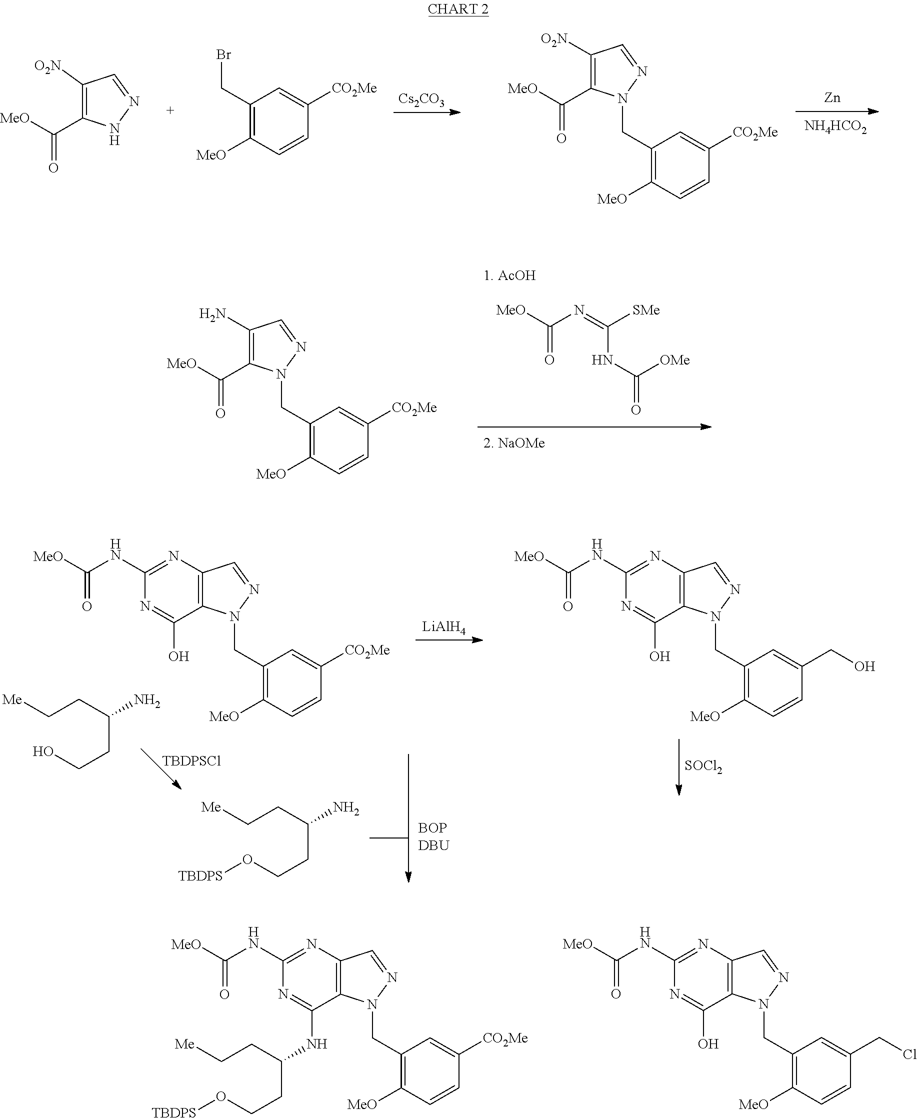

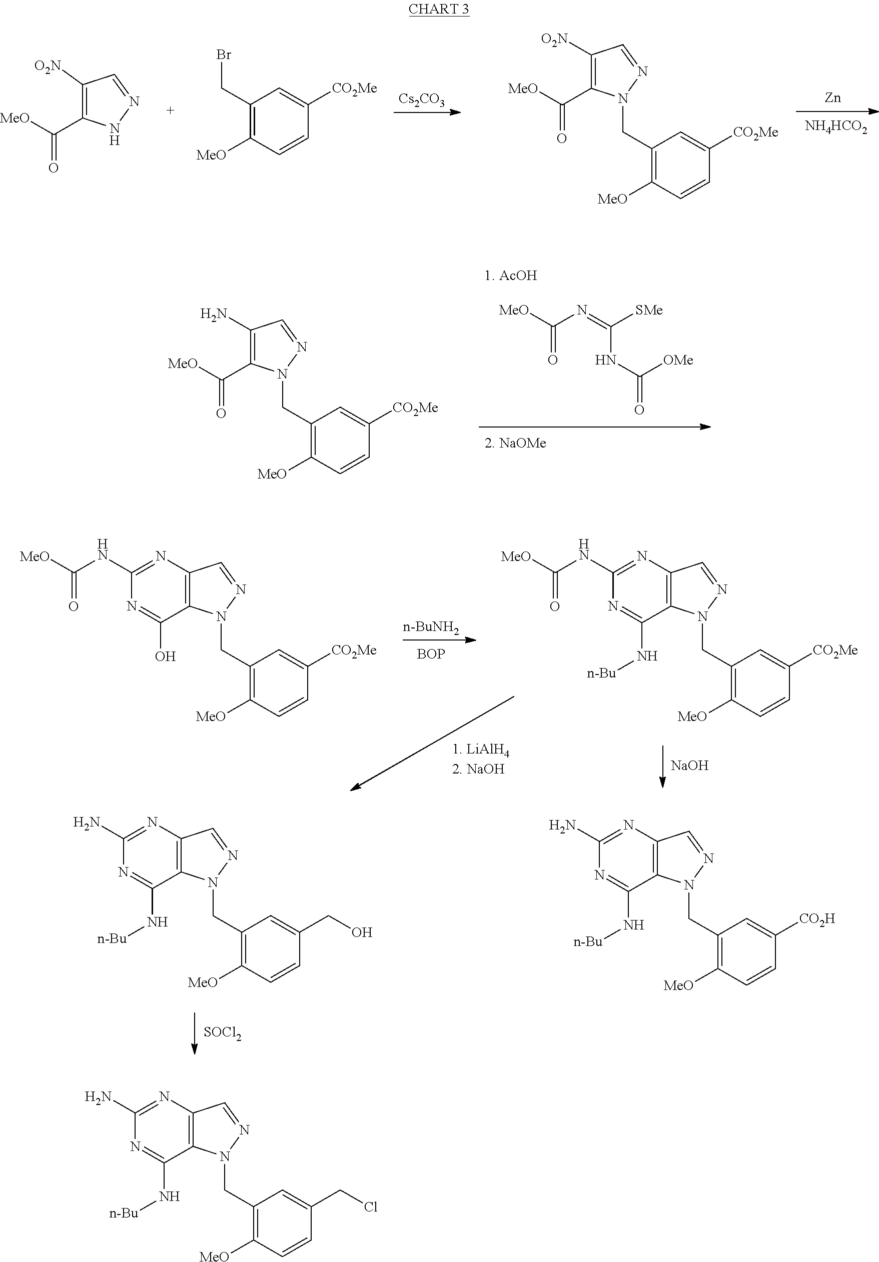

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×->1× in H₂O, warm at 37° C.; Cat #BD 558049) and kept the perm buffer (on ice) for later use.

For surface markers staining (CD69): prepared surface Abs: 0.045 ul hCD14-FITC (ThermoFisher Cat #MHCD1401)+0.6 ul hCD19-ef450 (ThermoFisher Cat #48-0198-42)+1.5 ul hCD69-PE (cat #BD555531)+0.855 ul FACS buffer. Added 3 ul/well, spin1000 rpm for 1 min and mixed on shaker for 30 sec, put on ice for 30 mins. Stop stimulation after 30 minutes with 70 uL of prewarmed 1× fix/lysis buffer and use Feliex mate to resuspend (15 times, change tips for each plate) and incubate at 37C for 10 minutes.

Centrifuge at 2000 rpm for 5 minutes aspirate with HCS plate washer, mix on shaker for 30 sec and then wash with 70 uL in dPBS and pelleted 2×s (2000 rpm for 5 min) and 50 ul wash in FACS buffer pelleted 1×s(2000 rpm for 5 min). Mix on shaker for 30 sec. For Intracellular markers staining (MX-1): Add 50 ul of BD Perm buffer III and mix on shaker for 30 sec. Incubate on ice for 30 minutes (in the dark). Wash with 50 uL of FACS buffer 2× (spin @2300 rpm×5 min after perm) followed by mixing on shaker for 30 sec. Resuspended in 20 ul of FACS buffer containing MX1 antibody ( ) (4812)-Alexa 647: Novus Biologicals #NBP2-43704AF647) 20 ul FACS bf+0.8 ul hlgG+0.04 ul MX-1. Spin 1000 rpm for 1 min, mix on shaker for 30se and the samples were incubated at RT in the dark for 45 minutes followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat #K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan® real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

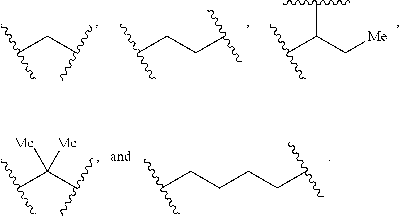

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (or “bicycloalkylene”) and “spiroalkanediyl” (or “spiroalkylene”) refer to divalent counterparts of a bicycloalkyl and spiroalkyl (or “spirocycloalkyl”) group. By way of illustration, an example of a

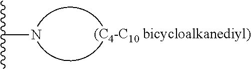 [SMILES: *N]

moiety is

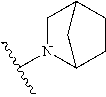 [SMILES: *N1CC2CCC1C2]

and an example of a

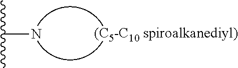 [SMILES: *N]

moiety is

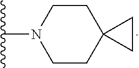 [SMILES: *N1CCC2(CC1)CC2]

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —N HC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alk yl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —N HC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —N HC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

[symbol]

In the formulae of this specification, a wavy line ( [symbol] ) transverse to a bond or an asterisk (*) at the end of the bond denotes a covalent attachment site. For instance, a statement that R is

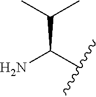 [SMILES: *[C@@H](N)C(C)C]

or that R is

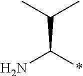 [SMILES: *[C@@H](N)C(C)C]

in the formula

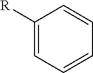

means

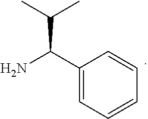

In the formulae of this specification, a bond traversing an aromatic ring between two carbons thereof means that the group attached to the bond may be located at any of the positions of the aromatic ring made available by removal of the hydrogen that is implicitly there (or explicitly there, if drawn out). By way of illustration, the formula

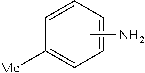

represents

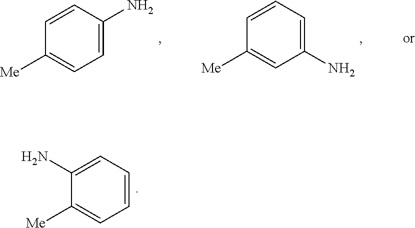 [SMILES: Cc1ccc(N)cc1 ; Cc1cccc(N)c1 ; Cc1ccccc1N]

In other illustrations,

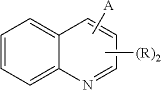

represents

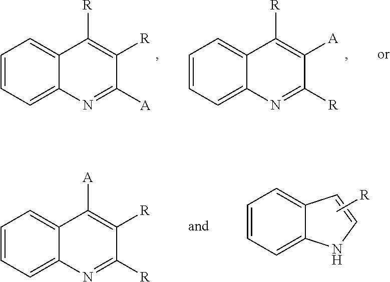

represents

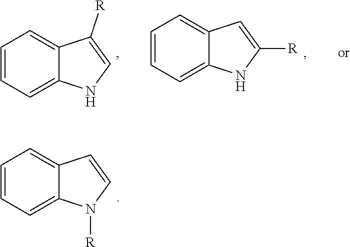

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### Acronyms and Abbreviations

This is a list of acronyms and abbreviations used in this specification, along with their meanings.

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig's base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | Hexafluorophosphate Azabenzotriazole |
| | Tetramethyl Uronium; |
| | 1-[Bis(dimethylamino)- |
| | methylene]-1H-1,2,3-triazolo[4,5-b] |
| | pyridinium 3-oxide hexafluorophosphate |
| HEPES | 4-(2-Hydroxyethyl)piperazine- |
| | 1-ethanesulfonic acid, |
| | N-(2-Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, | Liquid chromatography-mass spectrometry |
| LC/MS | |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context | Retention time, in min |
| of liquid | |
| chromatography) | |
| RT (in the context | Room (ambient) temperature, circa 25° C. |
| of reaction | |
| conditions) | |
| Sat. | Saturated |
| Soln | Solution |
| TBDPS | tert-Butyldiphenylsilyl |
| TBS | t-Butyldimethylsilyl group |
| TEA | Triethylamine |
| TEAA | Triethylammonium acetate |
| TFA | Trifluoroacetic acid |
| THF | Tetrahydrofuran |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat′l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0038403 A1 (2020) [2020a].
Poudel et al., US 2020/0039986 A1 (2020) [2020b].
Purandare et al., WO 2019/209811 A1 (2019).
Pryde, U.S. Pat. No. 7,642,350 B2 (2010).
Sato-Kaneko et al., JCI Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., US 2014/0141033 A1 (2014).
Young et al., U.S. Pat. No. 10,457,681 B2 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I 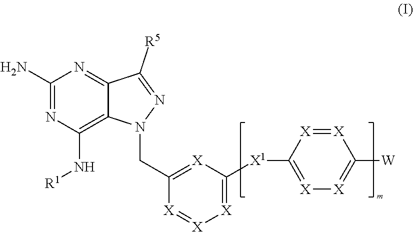
wherein
W is H, halo, C₁-C₃ alkyl, CN, (C₁-C₄ alkanediyl)OH, 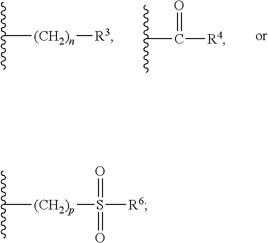
each X is independently N or CR²;
X¹ is O, CH₂, NH, S, or N(C₁-C₃ alkyl);
R¹ is (C₁-C₅ alkyl),
(C₂-C₅ alkenyl),
(C₁-C₅ alkanediyl)₀₋₁(C₃-C₆ cycloalkyl),
(C₁-C₅ alkanediyl)₀₋₁(C₅-C₁₀ spiroalkyl),
(C₂-C₅ alkanediyl)OH,
(C₂-C₅ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₅ alkanediyl)N[C(═O)](C₁-C₃ alkyl),
or
(C₂-C₅ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R³ is H, halo, OH, CN,
NH₂,
NH[C(═O)]₀₋₁(C₁-C₅ alkyl),
N(C₁-C₅ alkyl)₂,
NH[C(═O)]₀₋₁(C₁-C₄ alkanediyl)₀₋₁(C₃-C₅ cycloalkyl),
NH[C(═O)]₀₋₁(C₁-C₄ alkanediyl)₀₋₁(C₄-C₁₀ bicycloalkyl),
NH[C(═O)]₀₋₁(C₁-C₄ alkanediyl)₀₋₁(C₅-C₁₀ spiroalkyl),
N(C₃-C₆ cycloalkyl)₂,
O(C₁-C₄ alkanediyl)₀₋₁(C₃-C₅ cycloalkyl),
O(C₁-C₄ alkanediyl)₀₋₁(C₄-C₅ bicycloalkyl),
O(C₁-C₄ alkanediyl)₀₋₁(C₅-C₁₀ spiroalkyl),
O(C₁-C₄ alkanediyl)₀₋₁(C₁-C₆ alkyl),
N[C₁-C₃ alkyl]C(═O)(C₁-C₆ alkyl),
NH(SO₂)(C₁-C₅ alkyl),
NH(SO₂)(C₁-C₄ alkanediyl)₀₋₁(C₃-C₅ cycloalkyl),
NH(SO₂)(C₁-C₄ alkanediyl)₀₋₁(C₄-C₁₀ bicycloalkyl),
NH(SO₂)(C₁-C₄ alkanediyl)₀₋₁(C₅-C₁₀ spiroalkyl),
a 6-membered aromatic or heteroaromatic moiety,
a 5-membered heteroaromatic moiety, or
a moiety having the structure 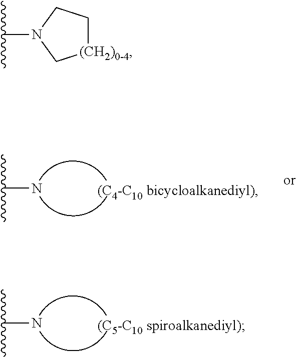
R⁴ is NH₂,
NH(C₁-C₅ alkyl),
N(C₁-C₅ alkyl)₂,
NH(C₁-C₄ alkanediyl)₀₋₁(C₃-C₅ cycloalkyl),
NH(C₁-C₄ alkanediyl)₀₋₁(C₄-C₁₀ bicycloalkyl),
NH(C₁-C₄ alkanediyl)₀₋₁(C₅-C₁₀ spiroalkyl),
N(C₃-C₆ cycloalkyl)₂,
or
a moiety having the structure 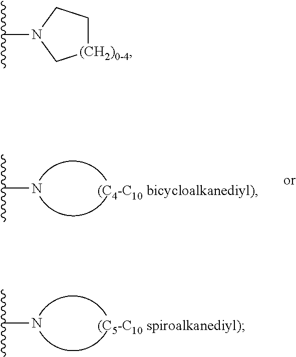
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 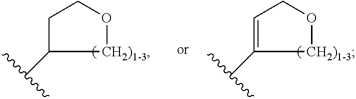
R⁶ is NH₂,
(NH)₀₋₁(C₁-C₅ alkyl),
N(C₁-C₅ alkyl)₂,
(NH)₀₋₁(C₁-C₄ alkanediyl)₀₋₁(C₃-C₅ cycloalkyl),
(NH)₀₋₁(C₁-C₄ alkanediyl)₀₋₁(C₄-C₁₀ bicycloalkyl),
(NH)₀₋₁(C₁-C₄ alkanediyl)₀₋₁(C₅-C₁₀ spiroalkyl),
N(C₃-C₆ cycloalkyl)₂,
or
a moiety having the structure 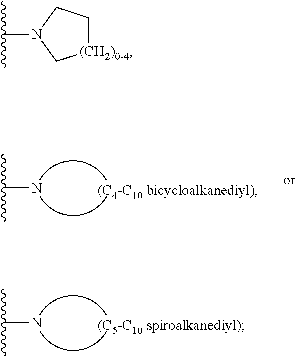
R^{x} and R^{y} are independently H or C₁-C₃ alkyl or R^{x} and R^{y} combine with the nitrogen to which they are bonded to form a 3- to 7-membered heterocycle;
m is 0 or 1;
n is 1, 2, or 3;
and
p is 0, 1, 2, or 3;
wherein in R¹, R², R³, R⁴, R⁵, and R⁶
an alkyl, cycloalkyl, alkanediyl, bicycloalkyl, spiroalkyl, cyclic amine, 6-membered aromatic or heteroaromatic moiety, 5-membered heteroaromatic moiety or a moiety of the formula 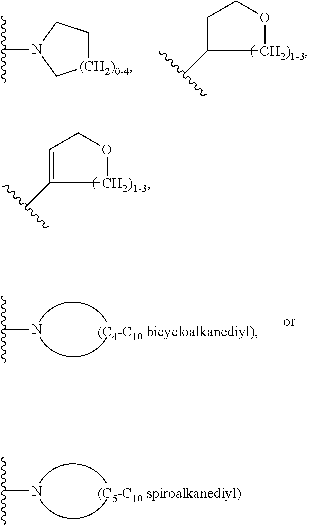
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, bicycloalkyl, spiroalkyl, or a moiety of the formula 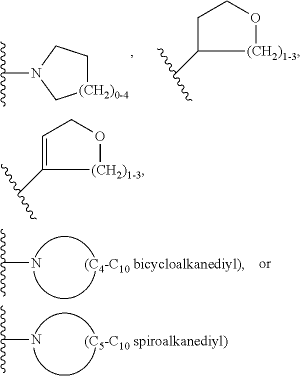
may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)₀₋₁CF₃,
or
N[C(═O)]₀₋₁(C₁-C₄ alkanediyl)₀₋₁(C₃-C₈ cycloalkyl).

2. A compound according to claim 1, wherein, in formula (I),

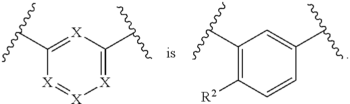

3. A compound according to claim 1, having a structure according to formula (Ia):

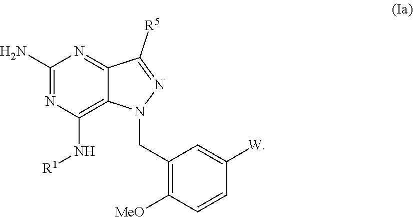

4. A compound according to claim 1, having a structure according to formula (Ib):

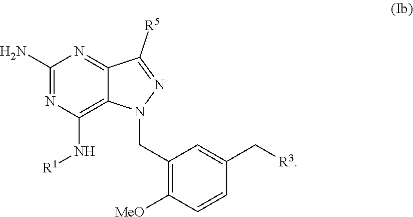

5. A compound according to claim 4, wherein R¹ is

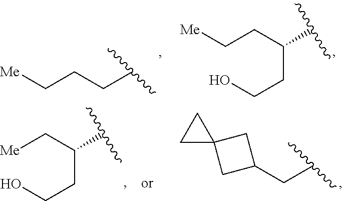

6. A compound according to claim 5, wherein R³ is 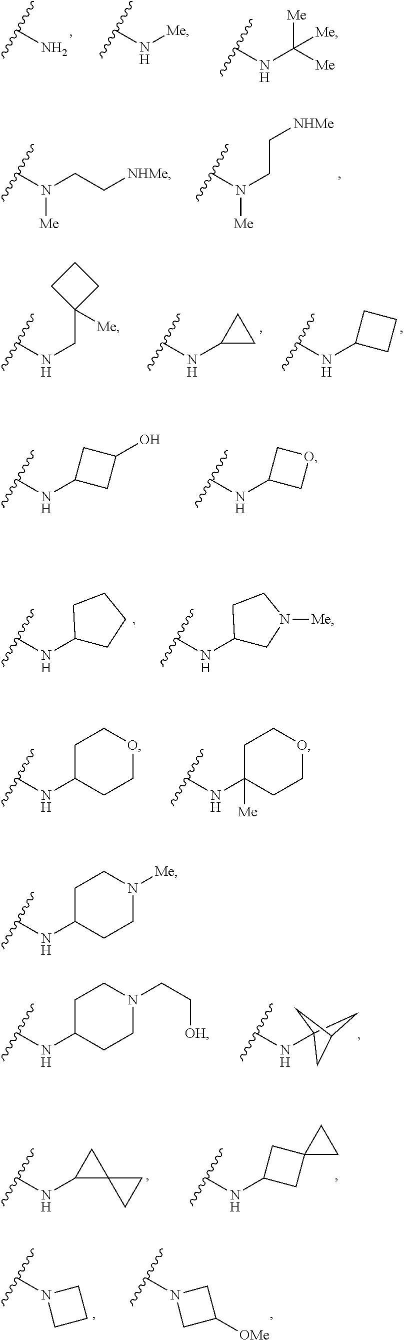 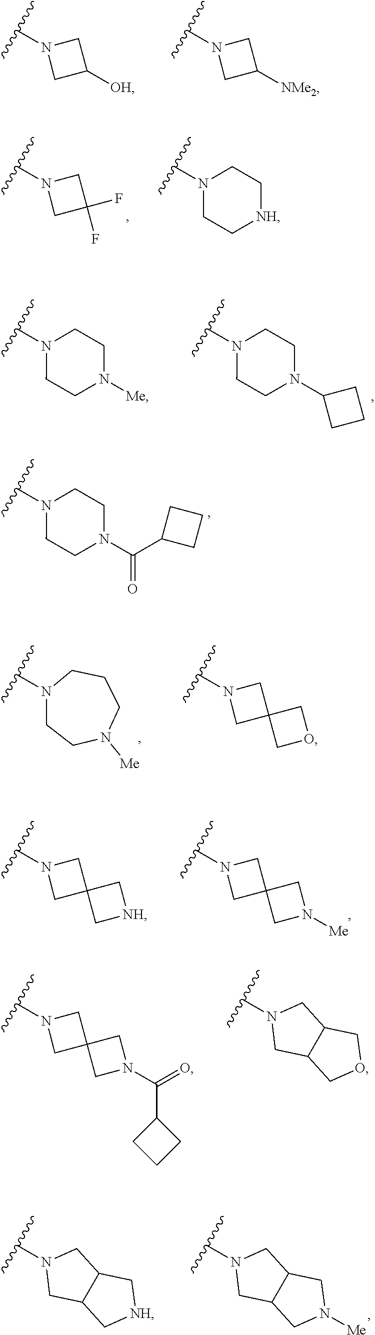 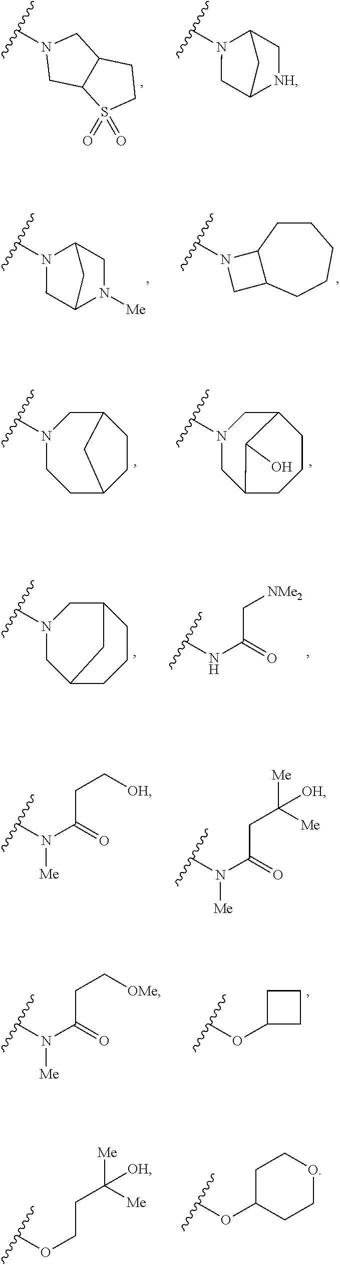 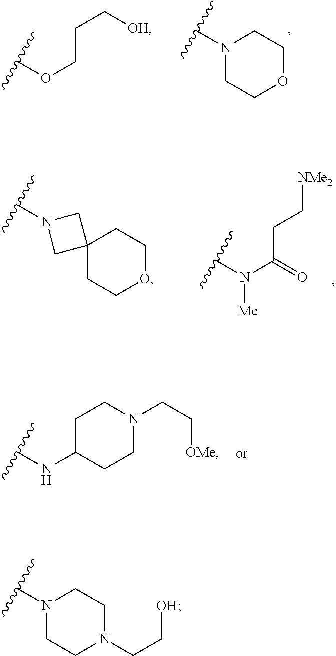 [SMILES: *OCCCO ; *N1CCOCC1 ; *N1CC2(CCOCC2)C1 ; *N(C)C(=O)CCN(C)C ; *NC1CCN(CCOC)CC1 ; *N1CCN(CCO)CC1]
and R⁵ is H or Me.

7. A compound according to claim 1, having a structure according to formula (Ic):

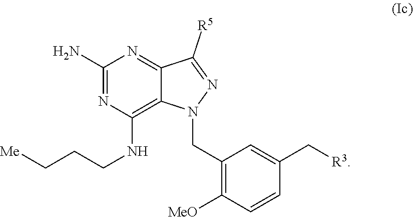

8. A compound according to claim 1, having a structure according to formula (Id):

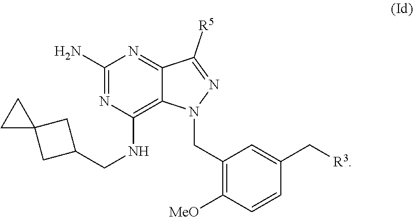

9. A compound according to claim 1, having a structure according to formula (Ie):

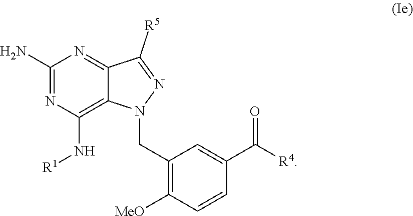

10. A compound according to claim 9, wherein R¹ 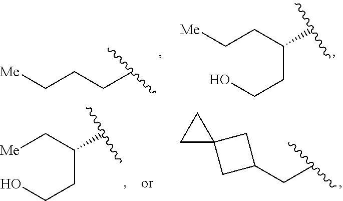
R⁴ is 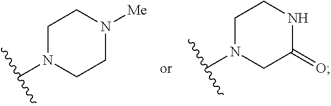 [SMILES: *N1CCN(C)CC1 ; *N1CCNC(=O)C1]
and R⁵ is H or Me.

11. A compound having a structure according to formula (If) 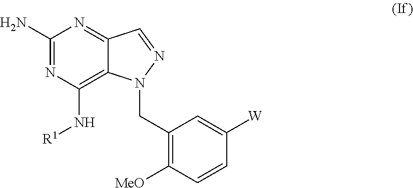
wherein
R¹ is 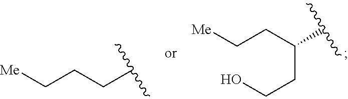 [SMILES: *CCCC ; *[C@@H](CCC)CCO]
and
W is

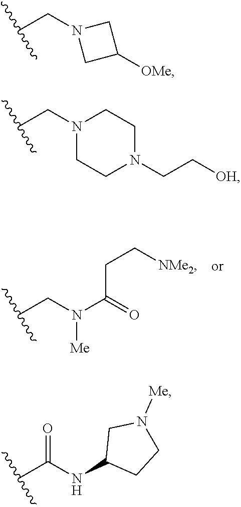

12. A method of treating a cancer, comprising administering to a patient suffering from such cancer a therapeutically effective combination of an anti-cancer immunotherapy agent and a compound according to claim 1.

13. A method according to claim 12, wherein the anti-cancer immunotherapy agent is an antagonistic anti-CTLA-4, anti-PD-1, or anti-PD-L1 antibody.

14. A method according to claim 12, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

15. A method according to claim 14, wherein the anti-cancer immunotherapy agent is ipilimumab, nivolumab, or pembrolizumab.

16. A compound according to claim 1, having a structure according to formula (Ig)

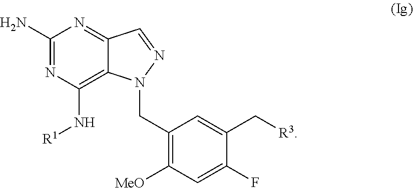

17. A compound according to claim 1, having a structure according to formula 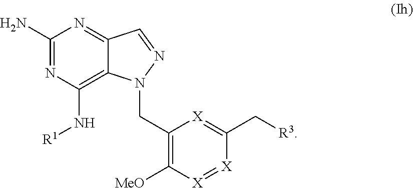
wherein one X is N and the other two are CH.

18. A method of treating a cancer, comprising administering to a patient suffering from such cancer a therapeutically effective combination of an anti-cancer immunotherapy agent and a compound according to claim 11.

19. A method according to claim 18, wherein the anti-cancer immunotherapy agent is an antagonistic anti-CTLA-4, anti-PD-1, or anti-PD-L1 antibody.

20. A method according to claim 18, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.
